(19) United States
(12) Patent Application Publication
(10) Pub. No.: US 2005/0036993 A1
(43) Pub. Date: Feb. 17, 2005

(54) Immune activation by double-stranded polynucleotides

(76) Inventors: Leonard D. Kohn, Bethesda, MD (US); Koichi Suzuki, North Bethesda, MD (US); Atsumi Mori, Bethesda, MD (US); Ken Iishi, Rockville, MD (US); Dennis M. Klinman, Patomac, MD (US); John M. Rice, West Chester, OH (US)

Correspondence Address: FROST BROWN TODD, LLC, 2200 PNC CENTER, 201 E. FIFTH STREET, CINCINNATI, OH, 45202, US

(21) Appl. No.: 10/830,898

(22) Filed: Apr. 22, 2004

Related U.S. Application Data
(63) Continuation of application No. 09/151,612, filed on Sep. 11, 1998, now abandoned.

(51) Int. Cl. G01N 33/53; A61K 48/00; C12N 15/85

(52) U.S. Cl. 424/93.21; 435/455

(57) ABSTRACT

Double-stranded polynucleotide activates the expression of immune recognition molecules. The polynucleotide can have a minimal length and activates the expression of molecules not encoded by a nucleotide sequence that is not necessarily related to the polynucleotide. The present invention provides for a simple and specific system to activate expression of Class I and/or Class II molecules of the major histocompatibility complex (MHC), and allows regulation of expression of MHC molecules on the cell-surface of antigen presenting cells and other immune cells. Also provided are systems for the screening, identification, and isolation of compounds that increase or decrease this activation.

### CROSS-REFERENCE TO RELATED APPLICATIONS

This is a continuation of application Ser. No. 09/151,612, filed Sep. 11, 1998, which is incorporated herein in its entirety by reference.

This invention was made in part with the support of the U.S. Government, which has a nonexclusive, nontransferable, irrevocable, paid-up license to practice or have practiced this invention for or on behalf of the United States throughout the world.

### BACKGROUND OF THE INVENTION

This invention relates to processes for inducing, preventing, or suppressing activation of major histocompatibility complex (MHC) class I and class II molecules, other molecules involved in antigen presentation and the immune recognition process, molecules controlling the growth and function of cells, and to the products identified for inhibiting, or enhancing, the processes. This allows manipulation of the immune system, particularly for conditions and diseases characterized as involving abnormal or aberrant regulation of the immune recognition system on normal cells, wherein they are converted to antigen presenting cells (APCs) and cause bystander activation of immune cells. This also allows manipulation of regulation of the immune recognition system on lymphocytes and antigen presenting cells of the host immune defense system. These processes are important for the development of immune response to viruses, bacteria, environmental agents which damage tissues, and oncogene-transformation. They are involved in the immune recognition process developing during gene therapy and vaccinations and are part of a normal host defense system. They coordinately control the growth, apoptosis, and function of cells to maintain the normal homeostatic balance of the cell driving the host defense process.

An important function of the immune system is to discriminate self from non-self antigens and to eliminate the latter. In addition, tolerance must be achieved so that the immune system does not attack itself or other normal tissues of the body. This recognition by the immune system involves complex cell-cell interactions and depends primarily on lymphocytes (e.g., B and T cells) and antigen-presenting cells (“APC”) (e.g., macrophages and dendritic cells).

The immune response is mediated by molecules encoded by the MHC which contains polymorphic genetic loci encoding an immune superfamily of structurally-and functionally-related products (D. P. Stites & A. I. Terr (eds), “Basic and Clinical Immunology,” Appelton and Lange, Norwalk, Conn./San Mateo, Calif., (1991)). Recognition by a lymphocyte, through its antigen-MHC receptor of antigen presented in a complex with MHC on the antigen-presenting cell, may then trigger an activation program in the lymphocyte and/or secretion of effector substances by the lymphocyte. The two principal classes of MHC molecules, Class I and Class II, each comprise a heterodimer of glycoproteins expressed on the cell surface. Class I molecules are found on virtually all somatic cell types, although they are expressed at different levels in different cell types. In contrast, Class II molecules are normally expressed only on a few cell types, such as lymphocytes, macrophages, and dendritic cells.

The Class I molecule is generally comprised of an MHC gene product (e.g., HLA-A, B and C loci encoding the heavy chain of Class I) and [symbol] 2-microglobulin, which is encoded by a non-MHC gene; the Class II molecule is generally comprised of two MHC gene products (e.g., HLA-DP, DQ and DR loci encoding I and [symbol] chains of Class II). Furthermore, non-covalently associated polypeptides (e.g., chaperone proteins and invariant chain) are encoded by non-MHC genes. Determination of the three-dimensional protein structure of MHC molecules by X-ray crystallography shows that although the genetic organizations of Class I and Class II genes are disparate, the protein structures of the different MHC molecules are similar with an antigen-binding pocket lined by polymorphic amino acid residues.

Antigens together with MHC molecules are presented to the immune system. (J. Klein & E. Gutze, “Major Histocompatibility Complex,” Springer Verlag, New York, 1977; E. R. Unanue, Ann. Rev. Immunology 2: 295-428, (1984)). For example, an endogenous antigen or a peptide sequence from a virus infecting a cell and expressing viral genes therein, may bind to the Class I molecule while exogenous antigen, e.g., a peptide sequence from an immunogen taken up by an antigen presenting cell and metabolized therein, may bind to the Class II molecule. The chemical structure of a peptide (e.g., length, amino acid composition, post-translational modification) will determine whether it can be processed and transported by the cell, and bound to the MHC molecule. Processing and transport of Class I related peptides involves, but is not limited to, proteasomes and transporters of antigen peptides (TAP) molecules among other cell organelles and proteins (I. A. York & K. L. Rock, Annu. Rev. Immunol. 14: 369-96 (1996)). Processing and expression of Class II related peptides involves, but is not limited to, invariant chain and HLA-DM molecules (J. Pieters, Curr. Opin. Immunol. 9: 89-96 (1997)). Controlling the cell-surface expression of an antigen-MHC complex by normal cells or regulating antigen-presenting cells at any point in the pathway producing such complexes (e.g., transcription, translation, post-translational modification, and folding of MHC polypeptides; production of peptide, which are able to bind an MHC molecule, from antigen through intracellular biosynthetic or degradative processes; transport of peptide into an organelle where binding to an “empty” MHC molecule can occur) will affect lymphocyte recruitment, maturation, differentiation, and activation through receptor-mediated recognition of the antigen-MHC complex.

CD4 is the receptor recognizing the Class II cell-surface molecule and CD4⁺T lymphocytes (usually helper T cells) recognize antigens presented in association with Class II gene products. CD8 is the receptor recognizing the Class I cell-surface molecule and CD8⁺T lymphocytes (usually cytotoxic T cells or CTL) recognize antigens in association with Class I gene products. In addition, co-receptors (e.g., CD28 or CTLA-4 on the lymphocyte, and CD80/B7-1 or CD86/B7-2 on the antigen presenting cell) will affect the activation status of an immune cell recognizing cognate antigen. Signalling through such receptors is integrated within the cell and determines the immune response of the individual cell, such as by secretion of substance that can mediate an immune response. Helper T cells are classified as Th1 or Th2 depending on the types of substances secreted during the immune response; those substances may promote the growth and/or differentiation of the target cell or immune cells recognizing the target cell. Cytotoxic T cells secrete compounds that may form pores in the target cell and degrade its contents. Thus cell-cell communication in the immune system may be accomplished through receptor-ligand interactions by cells in direct contact or at a distance.

It had been believed that Class I molecules function primarily as the targets of the cellular immune response, whereas Class II molecules regulate both the humoral (antibody mediated) and cellular immune response (J. Klein & E. Gutze, ibid. (1977)). MHC molecules have been the focus of much study with respect to research in autoimmune diseases because of their roles as mediators or initiators of the immune response. Class II molecules have been the primary focus of research in the etiology of autoimmune diseases, whereas Class I molecules have historically been the focus of research in transplantation rejection. But the present invention envisions a role for both classes of MHC molecule in host defense mechanism leading to autoimmunity.

Numerous experimental animal models for human disease have linked aberrant expression and/or function of MHC Class I and MHC Class II antigens to the autoimmune disease process, for example, insulin-dependent diabetes mellitus (IDDM) (Tisch and McDevitt, Cell 85: 291-297 (1996)), systemic lupus erythematosus (SLE) (Kotzin, Cell 85: 303-306 (1996)), uveoretinitis (Prendergast, et al., Invest. Opthalmol. Vis. Sci. 39: 754-762 (1998)), and Graves' disease (L. D. Kohn, et al., Intern. Rev. Immunol. 9: 135-165 (1992)), L. D. Kohn, et al., in Thyroid Immunity (D. Rayner and B. Champion (Eds.), R. G. Landes Biomedical Publishers, Austin/Georgetown, Tex., pp. 115-170 (1995)).

The pathological link between MHC Class I and/or Class II expression and disease has been examined in many of these model systems using a variety of biochemical and genetic approaches. One important piece of evidence for aberrant MHC gene function as a mediator of autoimmune disease stems from transgenic animal models in which the MHC genes have been inactivated. Using MHC Class I deficient animals, resistance to the autoimmune disease process and hence the dependence of autoimmunity upon MHC gene expression can be directly demonstrated in animal models for IDDM (Serreze, et al., Diabetes 43: 505-509 (1994)), and SLE (E. Mozes, et al., Science 261: 91-93 (1993)).

Systemic lupus erythematosus (SLE) is a chronic autoimmune disease that, like Graves' disease, has a relatively high rate of occurrence. SLE affects predominantly women, the incidence being 1 in 700 among women between the ages of 20 and 60 (A. K. Abbus, et al., (eds), “Cellular and Molecular Immunology,” W. B. Saunders Company, Philadelphia, pp. 360-370 (1991)). SLE is characterized by the formation of a variety of autoantibodies and by multiple organ system involvement (D. P. Stites & A. I. Terr, ibid, pp. 438-443 (1991)). Current therapies for treating SLE involve the use of corticosteroids and cytotoxic drugs, such as cyclosporin. Immunosuppressive drugs, such as cyclosporin, FK506 or rapamycin suppress the immune system by reducing T cell numbers and function (P. J. Morris, Curr. Opin. in Immun. 3: 748-751 (1991)). While these immunosuppressive therapies alleviate the symptoms of SLE and other autoimmune diseases, they have numerous severe side effects. In fact, extended therapy with these agents may cause greater morbidity than the underlying disease. A link between MHC Class I expression and SLE in animal models has been established. Thus, Class I deficient mice do not develop SLE in the 16/6 ID model (E. Mozes, et al., Science 261: 91-93 (1993)).

Diabetes Mellitus (DDM) is a disease characterized by relative or absolute insulin deficiency and relative or absolute glucagon excess (D. W. Foster, in J. B. Stanbury, et al., The Metabolic Basis of Inherited Disease, vol. 4, pp. 99-117 (1960)). Type I diabetes appears to require a permissive genetic background and environmental factors. Islet cell antibodies are common in the first months of the disease. They probably arise in part to [symbol] cell injury with leakage cell antigens but also represent a primary autoimmune disease. The preeminent metabolic abnormality in Type I diabetes is hyperglycemia and glucosuria. Late complications of diabetes are numerous and include increased atherosclerosis with attendant stroke and heart complications, kidney disease and failure, and neuropathy, which can be totally debilitating. The link to HLA antigens has been known since 1970. Certain HLA alleles are associated with increased frequency of disease, others with decreased frequency. Increased MHC Class I and aberrant MHC Class II expression in islet cells has been described (G. F. Bottazzo, et al., N. Eng. J. Med. 313: 353-360 (1985), Foulis and Farquharson, Diabetes 35: 1215-1224 (1986)). A definitive link to MHC Class I has been made in a genetic animal model of the disease. Thus, MHC Class I deficiency results in resistance to the development of diabetes in the NOD mouse (Serreze, et al., Diabetes 43: 505-509 (1994), L. S. Wicker, et al., Diabetes 43: 500-504 (1994)).

The dependence of the progressive multifocal inflammatory autoimmune disease phenotype exhibited by TGF-beta deficient transgenic mice (Shull, et al., Nature 359: 693-699 (1992); Kulkarni, et al., Proc. Natl. Acad. Sci. U.S.A. 90: 770-774 (1993); Boivin, et al., Am. J. Pathol. 146: 276-288 (1995)) on MHC Class II expression has recently been demonstrated using MHC Class II deficient animals. Specifically, TGF-beta deficient animals lacking MHC Class II expression are without evidence of inflammatory infiltrates, circulating antibodies, or glomerular immune complex deposits (Letterio, et al., J. Clin. Invest. 98: 2109-2119 (1996)).

Additional evidence supportive of MHC Class I and Class II antigens on target tissues as critical for the development of autoimmunity in animal models has been demonstrated in over-expression experiments.

Graves' disease (GD) is a relatively common autoimmune disorder of the thyroid. In Graves' disease, autoantibodies against thyroid antigens, particularly the thyrotropin receptor (TSHR), alter thyroid function and result in hyperthyroidism (D. P. Stites & A. I. Terr (eds), “Basic and Clinical Immunology,” Appleton and Lang, Norwalk, Conn./San Mateo, Calif., pp. 469-470 (1991)). Thyrocytes from patients with GD have aberrant MHC Class II expression and elevated MHC Class I expression (T. Hanafusa, et al., Lancet 2: 1111-1115 (1983); G. F. Bottazzo, et al., Lancet 2: 1115-1119 (1983); L. D. Kohn, et al., Int. Rev. Immunol. 912: 135-165 (1992)).

Numerous attempts to develop a GD model by immunizing animals with the extracellular domain of the thyrotropin receptor (TSHR) have largely failed (G. S. Seetharamaiah, et al., Autoimmunity 14: 315-320 (1993); S. Costagliola, et al., J. Mol. Endocrinol. 13: 11-21 (1994); S. Costagliola, et al., Biochem. Biophys. Res. Commun. 199: 1027-1034 (1994); S. Costagliola, et al., Endocrinology 135: 2150-2159 (1994); A. Marion, et al., Cell. Immunol. 158: 329-341 (1994); N. M. Wagle, et al., Autoimmunity 18: 103-108 (1994); G. Carayanniotis, et al., Clin. Exp. Immunol. 99: 294-302 (1995); G. S. Seetharamaiah, et al., Endocrinology 136: 2817-2824 (1995); N. M. Wagle, et al., Endocrinology 136: 3461-3469 (1995); H. Vlase, et al., Endocrinology 136: 4415-4423 (1995)). In most cases antibodies to the TSHR (TSHRAbs) which could inhibit TSH binding were produced and in some cases thyroiditis with a large lymphocytic infiltration developed (G. S. Seetharamaiah, et al., Autoimmunity 14: 315-320 (1993); S. Costagliola, et al., J. Mol. Endocrinol. 13: 11-21 (1994); S. Costagliola, et al., Biochem. Biophys. Res. Commun. 199: 1027-1034 (1994); S. Costagliola, et al., Endocrinology 135: 2150-2159 (1994); A. Marion, et al., Cell. Immunol. 158: 329-341 (1994); N. M. Wagle, et al., Autoimmunity 18: 103-108 (1994); G. Carayanniotis, et al., Clin. Exp. Immunol. 99: 294-302 (1995); G. S. Seetharamaiah, et al., Endocrinology 136: 2817-2824 (1995); N. M. Wagle, et al., Endocrinology 136: 3461-3469 (1995); H. Vlase, et al., Endocrinology 136: 4415-4423 (1995)). However, in no case did the immunization produce thyroid stimulating TSHRAbs which increase thyroid hormone levels, the hallmark of Graves,' nor were the morphologic or histologic features of the disease induced: glandular enlargement, thyrocyte hypercellularity, and thyrocyte intrusion into the follicular lumen. Further, in most studies (G. S. Seetharamaiah, et al., Autoimmunity 14: 315-320 (1993); S. Costagliola, et al., J. Mol. Endocrinol. 13: 11-21 (1994); S. Costagliola, et al., Biochem. Biophys. Res. Commun. 199: 1027-1034 (1994); S. Costagliola, et al., Endocrinology 135: 2150-2159 (1994); A. Marion, et al., Cell. Immunol. 158: 329-341 (1994); N. M. Wagle, et al., Autoimmunity 18: 103-108 (1994); G. Carayanniotis, et al., Clin. Exp. Immunol. 99: 294-302 (1995); G. S. Seetharamaiah, et al., Endocrinology 136: 2817-2824 (1995); N. M. Wagle, et al., Endocrinology 136: 3461-3469 (1995); H. Vlase, et al., Endocrinology 136: 4415-4423 (1995)) the antibodies that inhibited TSH binding were not shown to inhibit TSH activity mediated specifically by the TSH receptor, a feature characteristic of TSH binding inhibitory immunoglobulins (TBIIs) in GD (P. A. Ealey, et al., J. Clin. Endocrinol. Metab. 58: 909-914 (1984); A. Pinchera, et al., in Autoimmunity and the Thyroid, P. G. Walfish, et al., (Eds), Academic Press, New York, pp. 139-145 (1985); G. F. Fenzi, et al., in Thyroid Autoimmunity, A. Pinchera, et al., (Eds), Plenum Press, New York, pp. 83-90 (1987)).

These studies depended on the ability of the animal to process the TSHR as an extracellular antigen, rather than as a receptor in a finctional state on a cell. Several studies have implicated Class I as an important component in the development of autoimmune thyroid disease and in the action of methimazole (MMI), a drug used to treat GD (M. Saji, et al., J. Clin. Endocrinol. Metab. 75: 871-878 (1992); L. D. Kohn, et al., Intern. Rev. Immunol. 9: 135-165 (1992); E. Mozes, et al., Science 261: 91-93 (1993); D. S. Singer, et al., J. Immunol. 153: 873-880 (1994); L. D. Kohn, et al., in Thyroid Immunity, D. Rayner and B. Champion (Eds), R. G. Landes Biomedical Publishers, Texas, pp. 115-170 (1995)). In addition, aberrant Class II expression, as well as abnormal expression of Class I molecules, is evident on thyrocytes in autoimmune thyroid diseases (G. F. Bottazzo, et al., Lancet 2: 1115-119 (1983); G. F. Bottazzo, et al., N. Engl. J. Med. 313: 353-360 (1985); I. Todd, et al., Annals N.Y. Acad. Sci. 475: 241-249 (1986)), although the cause and role of aberrant Class II in disease expression was controversial (A. P. Weetman & A. M. McGregor, Endocrinol. Rev. 15: 788-830 (1994)).

The possibility that abnormal MHC expression, as well as a functional, full-length TSHR, might result in a Graves'-like disease, was tested by transfecting full-length human TSHR (hTSHR) into murine fibroblasts with or without aberrantly expressed Class II antigen (N. Shimojo, et al., Proc. Natl. Acad. Sci. U.S.A 93: 11074-11079 (1996); K.-I. Yamaguchi, et al., J. Clin. Endocrinol. Metab. 82: 4266-4269 (1997); S. Kikuoka, et al., Endocrinology 139: 1891-1898 (1998)). Mice immunized with fibroblasts expressing a Class II molecule and holoTSHR, but not either alone, could develop the major features characteristic of GD: thyroid-stimulating antibodies directed against the TSHR, increased thyroid hormone levels, an enlarged thyroid, and thyrocyte hypercellularity with intrusion into the follicular lumen. The mice additionally develop TBIIs, which inhibit TSH-increased cAMP levels in CHO cells stably transfected with the TSHR and appear to be different from the stimulating TSHR Abs, another feature of the humoral immunity in GD. Thus, by immunizing mice with fibroblasts transfected with the human TSHR and a MHC Class II molecule, but not by either alone, an induced immune hyperthyroidism was induced that has the major humoral and histological features of GD (N. Shimojo, et al., Proc. Natl. Acad. Sci. U.S.A 93: 11074-11079 (1996); K.-I. Yamaguchi, et al., J. Clin. Endocrinol. Metab. 82: 4266-4269 (1997); S. Kikuoka, et al., Endocrinology 139: 1891-1898 (1998)). The articles state that the results indicate that the aberrant expression of MHC Class II molecules on cells that express a native form of the TSHR can result in the induction of functional anti-TSHR antibodies that stimulate the thyroid. They additionally suggest that the acquisition of antigen-presenting ability on a target cell containing the TSHR can activate T and B cells normally present in an animal and induce a disease with the major features of autoimmune Graves'.

Another source of evidence for the importance of abnormal expression of MHC Class I and Class II in causing autoimmune disease derives from studies with drugs. Thionamide therapy has historically been used to treat GD. The most commonly used thionamides are methimazole, carbimazole and propylthiouracil. These thionamides contain a thiourea group; the most potent are thioureylenes (W. L. Green, in Werner and Ingbar's “The Thyroid”: A Fundamental Clinical Text, 6th Edition, L. Braverman & R. Utiger (Eds), J. B. Lippincott Co., p. 324 (1991)). The basis for thionamide therapy has, however, not focused on immune suppression. Rather, the basis had been suppression of thyroid hormone formation. Experiments suggesting an effect on immune cells, to inhibit antigen presentation or antibody formation, are largely discounted as nonphysiologic in vitro artifacts of high MMI concentration. MMI activity under those circumstances is suggested to be based on free-radical scavenger activity (D. S. Cooper, in Werner E. Ingbar's “The Thyroid”, op. cit., pp. 712-734 (1991)).

PCT Application WO 92/04033, Faustman, et al., identifies a method for inhibiting rejection of transplanted tissue in a recipient animal by modifying, eliminating, or masking the antigens present on the surface of the transplanted tissue. Specifically, this application suggests modifying, masking or eliminating human leukocyte antigen (HLA) Class I antigens. The preferred masking or modifying drugs are F(ab)' fragments of antibodies directed against HLA-Class I antigens. However, the effectiveness of such a therapy will be limited by the hosts' immune response to the antibody serving as the masking or modifying agent. In addition, in organ transplantation, this treatment would not affect all of the cells because of the perfusion limitations of the masking antibodies. Faustman, et al., contends that fragments or whole viruses can be transfected into donor cells, prior to transplantation into the host, to suppress HLA Class I expression. However, use of whole or fragments of virus presents potential complications to the recipient of such transplanted tissue since some viruses, SV40 in particular, can increase Class I expression (D. S. Singer & J. Maguire, Crit. Rev. Immunol. 10: 235-237 (1991)).

British patent 592,453, Durant, et al., identifies isothiourea compositions that may be useful in the treatment of autoimmune diseases in host versus graft (HVG) disease and assays for assessing the immunosuppressive capabilities of these compounds. The British patent does not describe methimazole or the suppression of MHC Class I molecules in the treatment of autoimmune diseases. Additionally, several autoimmune diseases have been treated with methimazole with potential success. In one study, MMI was deemed as good as cyclosporin in treating juvenile diabetes (W. Waldhausl, et al., Akt. Endokrin. Stoffw. 8: 119 (1987). U.S. Pat. No. 5,556,754, Singer et al. (which is equivalent to PCT Application WO 94/28897), issued Sep. 17, 1996, describes a method for treating autoimmune diseases using methimazole, methimazole derivatives and methimazole analogs. U.S. Pat. No. 5,310,742, Elias, issued May 10, 1994, describes the use of thioureylene compounds to treat psoriasis and autoimmune diseases. Propylthiouracil, methimazole, and thiabendazole are the only specific compounds disclosed in the patent.

It has now been found (L. D. Kohn, et al., Methimazole derivatives and tautomeric cyclic thiones to treat autoimmune diseases. U.S. patent application submitted Aug. 31, 1998, which is herein incorporated by reference in its entirety) that a specific class of methimazole derivatives and tautomeric cyclic thiones are effective in treating autoimmune diseases and suppressing the rejection of transplanted organs, and that these compounds show clear and unexpected benefits over the use of methimazole itself. In particular, these compounds: (a) are more effective in inhibiting basal and IFN-induced Class I RNA expression and in inhibiting KIFN-induced Class II RNA expression than methimazole; (b) inhibit the action of IFN and abnormal MHC expression by acting on the CIITAJY-box regulatory system; and (c) exhibit therapeutic activities in vivo. Specifically they inhibit development of SLE in the (NZBxNZW)F₁ mouse model and diabetes in the NOD mouse model, both of which are linked to abnormal expression of MHC genes.

In sum, the development of tissue-specific autoimmune diseases is associated with abnormal or aberrant expression of MHC molecules, Class I and/or Class II, on the surface of cells in the target tissue (G. F. Bottazzo, et al., Lancet 2: 1115-1119 (1983); I. Todd, et al., Annals N.Y. Acad. Sci. 475: 241-249 (1986); J. Guardiola & A. Maffei, Crit. Rev. Immunol. 13: 247-268 (1993); D. S. Singer, et al., Crit. Rev. Immunol. 17: 463-468 (1997)). Abnormal expression of MHC molecules on these non-immune cells can cause them to mimic antigen presenting cells and present self-antigens to T cells in the normal immune cell repertoire (M. Londei, et al., Nature 312: 639-641 (1984); N. Shimojo, et al., Proc. Natl. Acad. Sci. U.S.A. 93: 11074-11079 (1996)). This leads to a loss in self tolerance and the development of autoimmunity (G. F. Bottazzo, et al., Lancet 2: 1115-1119 (1983); I. Todd, et al., Annals N.Y. Acad Sci 475: 241-249 (1986); J. Guardiola & A. Maffei, Crit. Rev. Immunol. 13: 247-268 (1993); D. S. Singer, et al., Crit. Rev. Immunol. 17: 463-468 (1997); M. Londei, et al., Nature 312: 639-641 (1984); N. Shimojo, et al., Proc. Natl. Acad. Sci. U.S.A. 93: 11074-11079 (1996)). Prior to the present invention, there was, however, no comprehensive explanation as to how abnormal or aberrant MHC expression might develop in the target tissue, or how this might contribute to the ensuing immune cell responses involved in autoimmunity.

Viral infections can ablate self-tolerance, mimic immune responses to self antigens, and be associated with autoimmune disease (J. Guardiola & A. Maffei, Crit. Rev. Immunol. 13: 247-268 (1993); R. Gianani & N. Sarvetnick, Proc. Natl. Acad. Sci. U.S.A. 93: 2257-2259 (1996); M. S. Horowitz, et al., Nature Med 4: 781-785 (1998); C. Benoist and D. Mathis, Nature 394: 227-228 (1998); H. Wekerle, Nature Med 4: 770-771 (1998)).

Rheumatoid Arthritis (RA), multiple sclerosis (MS) and insulin-dependent diabetes mellitus (IDDM) are diseases which, at first glance, seem to have little in common. Yet all three are inflammatory disorders that are credited with a common autoimmune etiology. The evidence that autoimmunity is involved in human IDDM, MS and RA is indirect. It relies on the following observations: (1) the character of the lesion, which is largely dominated by mononuclear inflitrates; (2) the underlying genetic susceptibility, which involves major histocompatibility (MHC) genes (and other genes too); and (3) the resemblance of the human disease to animal models where the pathology is known to be autoimmune in origin. A fourth possible line of evidence, namely the efficacy of immunomodulatory or immunosuppressive therapies, is unfortunately much weaker than one would like it to be in these diseases (H. Wekerle, Nature Med 4: 770-771 (1998)).

Several indirect arguments support the idea that microbial agents influence the occurrence or course of certain autoimmune diseases. For example, there is evidence linking autoimmune thyroid disease to viral and bacterial infections (Y. Tomer & T. Davies, Endocr. Rev. 14: 107-121 (1993)). The mechanism by which this might occur is unknown (Y. Tomer & T. Davies, Endocr. Rev. 14: 107-121 (1993)). It was known that Rous sarcoma virus, adenoviruses 12 and 2, and certain Gross viruses reduced expression of Class I: however, SV40 radiation leukemia virus (RadLV), and Moloney murine leukemia virus (MoMuLV) viruses can increase Class I MHC expression (D. S. Singer & J. E. Maguire CRC Crit. Rev. Immunol. 10: 235-257 (1990)).

Other indirect evidence includes the fact that migrant populations acquire the disease prevalence of the geographical area to which they move, a prevalence correlated with latitude; that the incidence or frequency of autoimmune diseases has dramatically changed in the last two centuries; and that non-obese-diabetic (NOD) mice are protected from diabetes by bacterial infections. The nature of the agents involved and their mechanism of action remain unclear.

One mechanism by which a viral infection could ablate self-tolerance is the induction of interferon (IFN) production by an immune cell (I. Todd, et al., Annals N.Y. Acad. Sci. 475: 241-249 (1986); J. Guardiola & A. Maffei, Crit. Rev. Immunol. 13: 247-268 (1993); D. S. Singer, et al., Crit. Rev. Immunol. 17: 463-468 (1997); R. Gianani & N. Sarvetnick, Proc. Natl. Acad. Sci. U.S.A. 93: 2257-2259 (1996)). KIFN can certainly increase MHC gene expression in the target tissue (J. P-Y. Ting & A. S. Baldwin, Curr. Opin. Immunol. 5: 8-16 (1993)).

A wealth of genetic, biochemical and animal model data support a contributory role of inflammatory cytokines (e.g., IL-12, IL-18; and particularly KIFN) in the autoimmune process (Sarvetnick, J. Clin. Invest. 99: 371-372 (1997)). Studies using non-obese diabetic (NOD) mice, which spontaneously develop auto-immune diabetes reminiscent of Type I human IDDM, are particularly illustrative in demonstrating how KIFN stimulated processes play critical roles in the development of autoimmunity; and how the actions of other pro-inflammatory cytokines are channeled through KIFN stimulated processes—among which are the enhanced expression of MHC Class I and MHC Class II antigens.

IL-12 and IL-18 (KIFN inducing factor) are known to act synergistically in stimulating production of KIFN in T cells (Micallef, et al., Eur. J. Immunol. 26: 1647-1651 (1996)). In diabetic NOD mice the systemic expression of IL-18 (Roghe, et al., J. Autoimmun. 10: 251-256 (1997)) and islet expression of IL-12 are increased (Rabinovitch, et al., J. Autoimmun. 9: 645-651 (1996)). Moreover, additional IL-12 accelerates autoimmune diabetes in NOD mice (Trembleau, et al., J. Exp. Med. 181: 817-821 (1995)). Genetic analysis has determined the IL-18 gene maps to a near a non-MHC IDDM susceptibility gene (Idd2) associated with a genetic susceptibility for autoimmune diabetes (Kothe, et al., J. Clin. Invest. 99: 469-474 (1997)). These reports help to define a critical role for KIFN in the process of autoimmunity.

The role of KIFN in the autoimmune process is further substantiated by studies where KIFN's signaling capacity was abrogated in some manner. For example, transgenic NOD mice deficient in the cellular receptor for KIFN (Wang, et al., Proc. Natl. Acad. Sci. U.S.A. 94: 13844-13849 (1997)) do not develop autoimmune diabetes. NOD mice treated with a neutralizing antibody for KIFN (Debray-Sachs, et al., J. Autoimmun. 4: 237-248 (1991)) also do not develop autoimmune diabetes. While it is somewhat surprising that the onset of diabetes is only delayed in transgenic NOD mice deficient in IFN-gamma (Hultgren, et al., Diabetes 45: 812-817 (1996)), this observation only further stresses the importance of blocking the KIFN signal and more importantly IFN-gamma stimulated downstream events for the effective prevention of autoimmunity in NOD mice.

Analogous observations have been made in animal models for SLE. Soluble KIFN receptor blocks disease in the (NZBXNZW)F₁ spontaneous autoimmune disease model for SLE (Ozmen, et al., Eur. J. Immunol. 25: 6-12 (1995)); uveitis, where the targeted expression of KIFN increases ocular inflammation (Geiger, et al., Invest. Opthanlmol. Vis. Sci. 35: 2667-2681 (1994)); and autoimmune gastritis, where neutralizing KIFN antibody blocks disease (Barret, et al., Eur. J. Immunol. 26: 1652-1655 (1996)). Moreover, in humans treatment with KIFN has been reported to be associated with the development of an SLE-like disease (Graninger, et al., J. Rheumatol. 18: 1621-1622 (1991)).

It is well recognized that KIFN increases MHC Class I and Class II expression in many tissues and thus is linked to the action of a coregulatory molecule, the Class II transactivator (Mach, et al., Ann. Rev. Immunol. 14: 301-331 (1996); Chang, et al., Immunity 4: 167-178 (1996); Steimle, et al., Science 265: 106-109 (1994); Steimle, et al., Cell 5: 646-651 (1995); Chang, et al., J. Exp. Med. 180: 1367-1374 (1994); Chin, et al. Immunity 1: 687-697 (1994); V. Montani, et al., Endocrinology 139: 280-289 (1998)). It is also known that methimazole (MMI) can inhibit IFN-increased Class I and Class II expression in thyroid (M. Saji, et al., J. Clin. Endocrinol. Metab. 75: 871-878 (1992); V. Montani, et al., Endocrinology 139: 290-302 (1998)). Also, it has been shown that MMI decreases expression of CIITA increased Class II expression and this appears to be related to the action of MMI to enhance Y box protein gene expression; the Y box protein suppresses Class II gene expression (V. Montani, et al., Endocrinology 139: 280-289 (1998)).

Invoking cytokines or KIFN as a cause of autoimmunity caused by viruses does not, however, address the mechanism by which a tissue or target cell viral infection induces immune cells to produce KIFN; nor is it reasonable that KIFN alone would cause autoimmunity, since its administration does not induce typical autoimmune disease (F. Schuppert, et al., Thyroid 7: 837-842 (1997)). Moreover, generalized KIFN production by immune cells cannot account for cell-specific autoimmunity, i.e., destruction of pancreatic [symbol] but not I cells in insulin-dependent diabetes mellitus or involvement of only thyroid follicular cells, not parafollicular C cells, in autoimmune Graves' disease (G. F. Bottazzo, et al., Lancet 2: 1115-1119 (1983); I. Todd, et al., Annals N. Y. Acad. Sci. 475: 241-249 (1986); N. Shimojo, et al., Proc. Natl. Acad. Sci. U.S.A. 93: 11074-11079 (1996); A. K. Foulis et al Diabetologia 30: 333-343 (1987)).

Another possibility for autoimmunity caused by viruses is immunological cross-reactivity between anti-pathogen and anti-self responses, i.e., molecular mimicry (H. Wekerle, Nature Med 4: 770-771 (1998); C. Benoist & D. Mathis, Nature 394: 227-228 (1998)).

The currently fashionable concept of molecular mimicry (M. B. Oldstone, et al., Cell 50: 818-820 (1987)) proposes that pathogens express a stretch of protein that is related in sequence or structure to a particular self-component. This pathogen-encoded epitope can be presented by the major histocompatibility complex and activate self-reactive T cells. Activation could occur because the T cell's antigen receptor has a higher affinity for the pathogen protein than for the self-component, or because T cells are more readily primed in the inflammatory context of an infection. Because primed and amplified T lymphocytes have a lower threshold for activation, they can now attack self-antigens that they previously ignored.

Still another alternative concept to explain the action of viruses is bystander activation which proposes that pathogens disturb self-tolerance without antigenic specificity coming into play. They can do this by provoking cell death and the release of cellular antigens or increasing their visibility or abundance; thereby attracting and potentiating antigen-presenting cells and by perturbing the cytokine balance through the inflammation associated with infection (C. Benoist & D. Mathis, Nature 394: 227-228 (1998)).

There is good evidence that molecular mimicry could operate. Relevant homologies between mammalian and pathogen sequences have been found. Experimental support has come from animals immunized with peptides containing such homologous motifs (R. S. Fujinami & M. B. Oldstone, Science 230: 1043-1045 (1985)) and transgenic mice in which a viral epitope is expressed on particular organs (P. Ohashi, et al., Cell 65: 305-317 (1991); M. B. Oldstone, et al., Cell 65: 319-331 (1991).

Coxsackie B virus, has been linked to autoimmune diabetes (IDDM). Sero-epidemiological evidence for an association is sketchy (P. M. Graves' et al. Diabetes 46: 161-168 (1997)), but attention has been drawn to the homology between determinants of the Coxsackie P2-C protein and glutamate decarboxylase (GAD), one of the autoantigens recognized in IDDM (T. M. Ellis & M. A. Atkinson, Nature Med 2: 148-153 (1996)). It is possible that Coxsackie virus infection could unleash autoreactivity to GAD and thereby provide IDDM.

If viruses activate pathogenic autoimmunity through molecular mimicry, they should not be able to do so if the immune repertoire is blind to cross-reactive epitopes. M. S. Horwitz et al., (Nature Med. 4: 781-785 (1998)) tested this possibility and the potential importance of virus-induced bystander activation by studying the BDC2.5 mouse model of diabetes. Most of the T cells in these transgenic mice are reactive against a naturally expressed pancreatic antigen that is distinct from GAD. When carried on the NOD genetic background, BDC2.5 mice show heavy infiltration of the pancreas by T cells; the local lesion is active, as shown by lymphocyte activation, division and programmed cell death, but a balance is somehow maintained such that complete destruction of insulin-producing cells is avoided for a longtime (I. André, et al., Proc. Natl. Acad. Sci. U.S.A. 93: 2260-2263 (1996)).

Horwitz and colleagues found that infection by Coxsackie B4 rapidly provoked diabetes in the transgenic mice, but not in non-transgenic littermates or in NOD animals, which show a less extensive pancreatic infiltration. This effect was at least to some degree virus-specific, because it did not occur after infection by lymphocytic choriomeningitis virus. Coxsackie B4 infects pancreatic cells, so the local inflammation that it provokes probably disturbs the immunoregulatory balance of autoreactive T cells in the vicinity (increased levels of antigen and pro-inflammatory cytokines).

This interpretation is consistent with a previous analysis from the Zinkernagel group (S. Ehl, et al., J. Exp. Med 185: 1241-1251 (1997)), using another transgenic system. They found that functional cytotoxic T cells could be elicited through bystander activation, but could not home to and destroy the pancreas, unlike T cells activated, in higher numbers, by recognition of cognate viral antigen. The results of Zhao et al. (S.-Z. Zhao, et al., Science 279: 1344-1347 (1998)), although interpreted in the context of molecular mimicry, also underscore the importance of local effects of pathogens. These authors found that T cells activated by a mimic from Herpes simplex virus could not provide corneal keratitis without a local, virus-induced lesion.

Ultimately, the conclusion is that the suspected connection between Coxsackie B virus and IDDM is linked to viral infection of the pancreas and bystander activation of a pre-existing, but controlled, immune system. Homology to GAD would be a coincidence (C. Benoist & D. Mathis, Nature 394: 227-228 (1998)). Although this could be overstating the case that can be made from the available data, it will be important to keep in mind these demonstrations of viral bystander effects. For example, therapeutic immunointervention focused on cross-reactive epitopes would be misguided if a pathogen's main contribution were bystander activation of dormant autoreactive cells (C. Benoist and D. Mathis, Nature 394: 227-228 (1998)).

In sum, there is evidence that viral triggering of diverse autoimmune diseases including rheumatoid arthritis, insulin-dependent diabetes, and multiple sclerosis is caused by local viral infection of the tissue not molecular mimicry. It is suggested this involves MHC genes, results in presentation of self-antigens, and induces bystander activation of the T cells; the mechanism for this is obscure, as is its relation to the immune cell cytokine/IFN response (H. Wekerle, Nature Med 4: 770-771 (1998); C. Benoist & D. Mathis, Nature 394: 227-228 (1998)).

The mammalian immune system also responds to bacterial infection. One means to do this is rapidly initiating an inflammatory reaction that limits the early spread of pathogens and facilitates the emergence of antigen-specific immunity. Microorganisms have evolved to avoid such recognition by altering their expression of protein and lipid products. Yet DNA is an indispensable and highly conserved component of all bacteria. Indeed, the genomes of otherwise diverse bacteria share DNA motifs that are rarely found in higher vertebrates. Recent studies suggest that immune recognition of these motifs may contribute to the host's innate inflammatory response.

Bacterial, but not mammalian DNA, can boost the lytic activity of NK cells and induce KIFN production, an effect attributed to palindromic sequences present in bacterial DNA (S. Yammamoto, el al., J. Immunol. 148: 4072-4076 (1992)). In addition, other investigators showed that bacterial DNA, especially when complexed to DNA-binding proteins, could induce B cell activation. To better define the size and composition of the relevant immunostimulatory motif(s), Krieg and colleagues examined the activity of a series of synthetic oligodeoxynucleotides (ODNs) (A. M. Krieg, et al., Nature 374: 546-548 (1995)). Optimal stimulation was observed when the ODN contained at least one non-methylated CpG dinucleotide flanked by two 5′ purines (optimally GpA) and two 3′ pyrimidines (optimally TpC or TpT). Immune stimulation persisted despite purine/purine or pyrimidine/pyrimidine replacements, even if these substitutions eliminated a palindromic sequence. Yet if either base pair of the CpG was eliminated, stimulatory activity was lost. Optimizing the flanking region or incorporating two CPGs into a single ODN increased stimulation. The minimal length of a stimulatory ODN was 8 bp. These findings established that immune stimulation was mediated by a six base pair nucleotide motif consisting of an unmethylated CpG dinucleotide flanked by two 5′ purines and two 3′ pyrimidines imbedded in a larger fragment of DNA (A. M. Krieg, et al., Nature 374: 546-548 (1995)). Such motifs are expressed nearly 20 times more frequently in bacterial than vertebrate DNA due to differences in the frequency of utilization and methylation pattern of CpG dinucleotides in prokaryotes versus eukaryotes.

Evidence suggests that these motifs act directly on cells of the immune system. Cells responsive to CpG ODN include macrophages, B lymphocytes, T lymphocytes, and NK cells. CpG ODN rapidly stimulate B cells to produce IL-6 and IL-12, CD4+ T cells to produce IL-6 and KIFN, and NK cells to produce KIFN both in vivo and in vitro (D. M. Klinman, et al., Proc. Natl. Acad. Sci. U.S.A. 93: 2879-2883 (1996)). This lymphocyte stimulation is polyclonal and antigen non-specific in nature, although specificity is retained with respect to the phenotype of cells activated and the type of cytokine they produced. The finding that NK and T cells as well as B cells are triggered by CpG-containing ODNs suggests that immune recognition of this motif is evolutionarily conserved among multiple types of immunologically active cells. Kinetic studies reveal that CpG ODNs induce cytokine release within four hours of administration, with peak production occurring by 12 hours (D. M. Klinman, et al., Proc. Natl. Acad. Sci. U.S.A. 93: 2879-2883 (1996)). Maximal cytokine production is observed using ODNs at a concentration of 0.10-0.33 ug/ml (D. M. Klinman, et al., Proc. Natl. Acad. Sci. U.S.A. 93: 2879-2883 (1996)). Synthetic ODN expressing stimulatory CpG motifs have been used as adjuvants to boost the immune response to DNA and protein based immunQgens. In vivo experiments demonstrate that CpG-containing oligos augment antigen-specific antibody production by up to ten fold, and KIFN production by up to six fold. For example, CpG ODN boost antigen-specific immune responses when co-administered with either protein- or DNA- based vaccines (Y. M. Sato, et al., Science 273: 352-354 (1996); M. E. Roman, et al., Nature Medicine 3: 849-854 (1997); D. M. Klinman, et al., J. Immunol. 158: 3635-3642 (1997)). This activity is present whether the motifs are intrinsic parts of the antigen (as in the backbone of a DNA vaccine), or co-administered along with the antigen (M. E. Roman, et al., Nature Medicine 3: 849-854 (1997)). However, immunogenicity is improved when the CpG oligo is physically linked to the relevant antigen. This is true both in the case of DNA vaccines and protein antigens. These results confirm the intuitive expectation that optimal stimulation occurs when antigen and adjuvant are presented to the immune system in close spatial and temporal sequence. These data suggest that CpG oligos initiate a complex cascade of events in vivo that may have broad application for immune regulation.

Saji, et al., (Proc. Natl. Acad. Sci. U.S.A. 89: 1944-1948 (1992)) described hormonal regulation of Class I genes in the rat thyroid cell line, FRTL-5. Treatment of the FRTL-5 cell line with thyroid-stimulating hormone (TSH) resulted in decreased transcription of Class I genes and reduced cell surface levels of Class I antigens. Saji, et al., (J. Clin. Endocrinol. Metab. 75: 871-878 (1992)) demonstrated that agents such as serum, insulin, insulin-like growth factor-I (IGF-1), hydrocortisone, and thyroid-stimulating thyrotropin receptor autoantibodies from Graves' patients decrease Class I gene expression in that FRTL-5 cells. In addition, treatment of the FRTL-5 cells with methimazole (MMI) or high doses of iodide resulted in decreased Class I gene expression. The effect of MMI on reduction of Class I expression was shown to be at the level of transcription and was additive with thyroid stimulating hormone and other hormones which normally suppress Class I in these cells. Saji, et al., (J. Clin. Endocrinol Metab. 75: 871-878 (1992)) suggested a mechanism by which MMI may act in the thyroid during treatment of GD; no extrapolation was made to any other autoimmune diseases. The use of MMI as an immunosuppressant has, however, been controversial.

The U.S.P. Dictionary (US Pharmacopeia, Rockville, Md., 1996) includes methimazole (CAS-60-56-0) and describes it as a thyroid inhibitor. U.S. Pat. No. Re. 24,505, Rimington, et al., reissued Jul. 22, 1958, discloses a group of imidazole compounds useful as anti-thyroid compounds.

Further, the action of MMI as an immunosuppressant is controversial. Thus, there have been differing reports on the ability of antithyroid drugs to suppress MHC Class II antigen expression in patients with Graves' disease (J. C. Carel, et al., in H. A. Drexhage & W. A. Weirsinga (Eds). The thyroid and autoimmunity. Excerpta Medica, Amsterdam, pp. 145-147 (1986); J. Aguayo, et al., J. Clin Endocrinol. Metab. 66: 903-908 (1988); T. F. Davies et al. Clin Endocrinol. 31:

125-135 (1989)) and concerns were expressed that there was an absence of dose dependencies on immunologic parameters in refractory Graves' patients treated with MMI before surgery (R. Paschke, et al., J. Clin Endocrinol. Metab. 80:

2470-2474 (1995)). D. S. Cooper (N. Engl. J. Med. 311: 1353-1362 (1984)) concluded that MMI was an effective therapeutic agent because of actions to block thyroid hormone formation and that its activity as an immunosuppressant might be an in vitro artifact.

Nevertheless, Methimazole has been used to treat autoimmune diseases other than those of the thyroid.

U.S. Pat. No. 5,310,742, Elias, issued May 10, 1994, describes the use of thioureylene compounds to treat psoriasis and autoimmune diseases. Propylthiouracil, methimazole, and thiabendazole are the only specific compounds disclosed in the patent. Examples show the use of methimazole to treat psoriasis in humans and the use of thioureylene to treat rheumatoid arthritis, lupus and transplant rejection. No methimazole analogs or derivatives are disclosed or discussed. No tautomeric cyclic thiones are disclosed or discussed.

U.S. Pat. No. 5,556,754, Singer et al. (which is equivalent to PCT Application W0 94/28897), issued Sep. 17, 1996, describes a method for treating autoimmune diseases using methimazole, methimazole derivatives and methimazole analogs. The terms “methimazole derivatives” and “methimazole analog” are not defined or exemplified anywhere in the patent.

In one study, MMI was deemed as good as cyclosporin in treating juvenile diabetes (W. Waldhausl, et al., Akt. Endokrin. Stoffw. 8: 119 (1987)).

U.S. Pat. No. 5,051,441, Matsumoto, et al., issued Sep. 24, 1991, discloses diphenyl imidazoline derivatives which are, said to act as immunomodulators, showing efficiency in the treatment of rheumatoid arthritis, multiple, sclerosis, systemic lupus, and rheumatic fever.

U.S. Pat. No. 5,202,312 Matsumoto, et al., issued Apr. 13, 1993, discloses imidazoline-containing peptides which are said to have immunomodulatory activity.

Methimazole and methimazole derivatives have, however, been reported to have activities other than as an antithyroid agent or immunosuppressive agent.

U.S. Pat. No. 4,148,885, Renoux, et al., issued Apr. 10, 1979, describes the use of specific low molecular weight sulfur-containing compounds as immunostimulants. Methimazole, thioguanine and thiouracil are among the compounds specified. No methimazole analogs or derivatives are disclosed or discussed. No tautomeric cyclic thiones are disclosed or discussed.

U.S. Pat. No. 5,010,092, Elfarra, issued Apr. 23, 1991, describes a method of reducing the nephrotoxicity of certain drugs via the coadministration of methimazole or carbimazole, (which is taught to be the pro-drug of methimazole) together with the nephrotoxic drug. No methimazole analogs or derivatives are discussed in this patent. No tautomeric cyclic thiones are disclosed or discussed.

U.S. Pat. No. 5,578,645, Askanazi, et al., issued Nov. 26, 1996, describes a method for minimizing the side effects associated with traditional analgesics. This is accomplished via the administration of a mixture of specific branched amino acids together with the analgesic compound. Methimazole is disclosed, in the background section of this patent, as a nonsteroidal anti-inflammatory drug which may provide some of the side effects which this invention is said to address. No tautomeric cyclic thiones are disclosed or discussed.

U.S. Pat. No. 5,587,369, Daynes, et al., issued Dec. 24, 1996, describes a method for preventing or reducing ischemia following injury. This is accomplished by introducing dehydroepiandrosterone (DHEA), DHEA derivatives, or DHEA congeners to a patient as soon as possible after the injury. The background section of this patent teaches that methimazole is a thromboxane inhibitor which has been shown to prevent vascular changes in bum wounds.

U.S. Pat. No. 4,073,905, Kummer, et al., issued Feb. 14, 1978, discloses 2-amino-4-phenyl-2-imidazolines, which are said to be useful for treating hypertension.

U.S. Pat. No. 3,390,150, Henry, issued Jun. 25, 1968, is representative of a group of patents which disclose nitroimidazole derivatives which possess antischistosomal and antitrichomonal activity.

U.S. Pat. No. 3,505,350, Doebel, et al., issued Apr. 7, 1970, discloses a group of substituted 2-mercaptoimidazole derivatives which are said to be effective as anti-inflammatory agents. Illustrative compounds include 1-(4-fluorophenyl)-5-methyl-2-mercaptoimidazole and 1-methyl-5-phenyl-2-mercaptoimidazole.

Methimazole, therefore, is known in the art for a variety of pharmaceutical utilities: for the treatment of psoriasis (Elias), as an immunostimulant (Renoux et al.), for the reduction of nephrotoxicity of certain drugs (Elfarra), for the minimization of side effects found with certain analgesics (Oskinasi et al.), as a thyroid inhibitor (U.S.P. Dictionary), and as a thromboxane inhibitor (Daynes et al.). It is also taught in the Singer et al. patent (U.S. Pat. No. 5,556,754), as being useful in the treatment of autoimmune diseases, such as rheumatoid arthritis and systemic lupus. While the Singer et al. patent (U.S. Pat. No. 5,556,754) contains general references to the use of methimazole analogs and derivatives for these therapeutic purposes, no definition of these compounds is given and no specific compounds are suggested.

It has recently been found (L. D. Kohn, et al., Methimazole derivatives and tautomeric cyclic thiones to treat autoimmune diseases. U.S. patent application submitted Aug. 31, 1998)) that a specific class of methimazole derivatives, tautomeric cyclic thiones, are effective in treating autoimmune diseases and suppressing the rejection of transplanted organs, and that these compounds show clear and unexpected benefits over the use of methimazole itself. In particular, these compounds: (a) are more effective in inhibiting basal and IFN-induced Class I RNA expression and in inhibiting IFN-induced Class II RNA expression than methimazole; (b) inhibit the action of IFN by acting on the CIITA/Y-box regulatory system; (c) may be significantly more soluble than methimazole, leading to significant formulation flexibility and advantages; (d) have less adverse effects on thyroid function than methimazole; (e) have an enhanced ability to bind to targets affected by MMI; and (f) exhibit therapeutic activities in vivo. These properties are unexpected based on the known properties of methimazole and particularly the tautomeric cyclic thiones.

Cyclic tautomeric thiones have not been described as immunoregulatory agents. Rather Kjellin and Sandstrom, Acta Chemica Scandinavica, 23: 2879-2887 and 2888-2899 (1969), disclosed a series of tautomeric cyclic thiones, i.e., oxazoline, thiazoline, and imidazoline-2-(3)-thiones having methyl and phenyl groups in the 4 and 5 positions. The compounds were used for a study of thione-thiol equilibria. No pharmaceutical, or any other utility, is disclosed or suggested for these compounds.

U.S. Pat. No. 3,641,049, Sandstrom, et al., issued Feb. 8, 1972, discloses N, N′-dialkyl-4-phenylimidazoline-2-thiones, particularly 1,3-dimethyl-4-phenylimidazoline-2-thione, for use as an antidepressant agent. The dimethyl compound is also said to exhibit antiviral properties against herpes simplex and vaccinia viruses.

It has been noted that specific viruses or viral promoters operably linked to nucleic acid inserts could increase Class I gene expression in cultured cells (D. S. Singer & J. E. Maguire, CRC Crit. Rev. Immunol. 10, 235-257 (1990)). Whether this might be related to a primary action of the virus on the target tissue to increase Class I and whether this might be the triggering effect on the cascade of events leading to an autoimmune response was determined as disclosed herein.

### SUMMARY OF THE INVENTION

It is demonstrated herein that the introduction of double-stranded nucleic acids into the cytoplasm of mammalian cells results in the increase the expression of immune response recognition molecules. This activation process transforms the affected cell into an APC capable of stimulating an immune response and may be the triggering event in autoimmunity; alternatively, or in addition, it may contribute to the activity of immune and antigen presenting cells normally present in the host. This natural response may also contribute to the pathogenesis of infectious diseases, chronic degenerative diseases and cancer. This discovery of a natural host defense response is exploited for the discovery of drugs and therapies for the treatment of these conditions and for the detection and diagnosis of the same. By artificially mimicking this activation process, systems for drug screening, drug target identification, immunization and diagnostic assays are enabled.

An object of this invention is the identification of drug compounds which can increase or decrease activation of immune recognition molecules.

Another object of this invention is to identify foreign or endogenous substances in an organism that induce, prevent, or suppress activation of immune recognition molecules in a target cell or tissue, in immune cells, or in antigen presenting cells.

Another object is to identify drug compounds and foreign or endogenous substances in an organism that enhance, prevent, or suppress growth and function of host cell or tissue when immune recognition molecules are increased or decreased by the invention disclosed herein.

Another object is to identify drug compounds and foreign or endogenous substances in an organism that induce, prevent or suppress viral activiation of host cell molecules in a target cell or tissue, in immune cells, or in antigen presenting cells.

Another object is to identify drug compounds and foreign or endogenous substances in an organism that induce, prevent or suppress bacterial activiation of host cell molecules in a target cell or tissue, in immune cells, or in antigen presenting cells.

Another object is to identify drug compounds and foreign or endogenous substances in an organism that induce, prevent or suppress activiation of host cell molecules caused by environmental damage to a target cell or tissue, immune cells, or antigen presenting cells.

Another object is to identify drug compounds and foreign or endogenous substances in an organism that enhance immune recognition by oncogene transformed target cells or tissue, immune cells, or antigen presenting cells.

Another object is to identify drug compounds and foreign or endogenous substances in an organism that enhance immune recognition by a target cell or tissue within an immunodeficient animal.

Another object is to identify drug compounds and foreign or endogenous substances in an organism that prevent or suppress oncogene activation of host cell molecules in a target cell or tissue, in immune cells, or in antigen presenting cells.

Another object is to identify drug compounds and foreign or endogenous substances in an organism that prevent or suppress immune responses associated with gene therapy in a target cell or tissue, in immune cells, or in antigen presenting cells.

A further object of this invention is the isolation of such compounds and substances. Thus products identified and/or isolated by this invention are also envisioned.

One additional use could be to prepare comparative cDNA or mRNA expression libraries for identification of differentially expressed genes in order to identify key genes or proteins which participate in the process and may serve as drug targets. The comparison would be between ds polynucleotide treated and untreated cells of various tissue types.

Another embodiment would be to assess active modulators of the “DNA response” as anti-infectives in in vitro models of viral, bacterial, and parasitic infections, in a two step drug discovery process.

The invention comprises introduction of a double-stranded polynucleotide into a cell to induce activation of at least one immune recognition molecule in or on the cell. The cell may be derived from any organism with an immune system, preferably a mammal. The cell is preferably a non-immune cell that is converted into a cell capable of presenting antigen to the immune system by the introduction of the double-stranded polynucleotide. The cell may, however, be typical of the immune system (e.g., lymphocytes, “professional” antigen presenting cells).

Introduction into the cell may be accomplished by, for example, entry of an infectious agent, phagocytosis, transfection, transformation, or leakage from a DNA-containing organelle. Thus the sequence of the polynucleotide is not necessarily related to any of the immune recognition molecules being activated.

Immune recognition molecules are those involved in antigen presentation such as, for example, MHC Class I and Class II molecules, peptide transporters, proteasome, HLA-DM, invariant chain, immunomodulators, kinases, phosphatases, signal transducers, and activators or coregulators of transcription. If the molecule is expressed on the cell surface, it may be conveniently detected by an antibody reacting to the intact cell or cell membranes. In any case, promoter activity of the gene, RNA transcripts of the molecule, and translation of the protein may be measured to detect expression of the immune recognition molecule. Expression may also be detected indirectly by bioassays that measure presentation of antigen and other processes involved in immune activation (e.g., release of soluble mediators of immunity, expression of receptors for the soluble mediators). Activation may also be measured by the cellular signals (e.g., tyrosine or serine/threonine phosphorylation, ADP ribosylation, proteolytic cleavage) generated during an immune response.

Increasing the ability of a cell to present antigen and activate the immune system by this invention allows its use as an activated APC. The activated APC may be introduced into an organism, preferably the activated APC is injected or surgically implanted into its own host organism (e.g., a murine cell into a mouse), to initiate an immune response. The immune response may be restricted to the MHC haplotype expressed on the activated APC. Presentation of an autoantigen may lead to development of autoimmunity, a tumor antigen may lead to an immune response against the tumor, or the immune response to a selected antigen presented by the activated APC may be used to immunize or tolerize against that antigen.

This invention provides a simple system to regulate expression of immune recognition molecules, and allows one to increase or decrease the amount of MHC molecules expressed on the cell surface of professional and nonprofessional antigen-presenting cells. By acting early in the pathway for generating antigen-MHC complexes, this invention can profoundly affect immunization, tolerization, and other biological processes dependent on activation of immune recognition molecules. Also provided are systems for the screening, identification, and isolation of compounds that suppress or enhance activation by decreasing or increasing, respectively, expression of immune recognition molecules.

The invention can be distinguished from the effects of CpG sequences because methylation does not alter activity whereas methylation eliminates CpG activity. There is no sequence specificity, whereas optimal CpG stimulation depends on sequence, e.g., when the ODN contains at least one non-methylated CpG dinucleotide flanked by two 5′ purines (optimally GpA) and two 3′ pyrimidines (optimally TpC or TpT). Most importantly, CpG motifs act directly only on cells of the immune system, whereas the ds nucleic acids described herein also work on nonimmune cells and convert them to APC.

The present invention may be used additively or synergistically with synthetic ODN expressing stimulatory CpG motifs, for example as adjuvants to boost the immune response to DNA and protein based immunogens and when coadministered with protein or DNA-based vaccines (Y. M. Sato, et al., Science 273: 352 (1996); M. E. Roman, et al., Nature Medicine 3: 849 (1997); D. M. Klinman, et al., J. Immunol. 158: 3635 (1997)). The one agent (ds nucleic acids) acts on the nonimmune cells to improve immune recognition; the other (CpG motifs) work on the immune cells to activate their responsiveness.

Examples of autoimmune diseases wherein this invention is relevant include, but are not limited to, rheumatoid arthritis, psoriasis, juvenile or type I diabetes, primary idiopathic myxedema, systemic lupus erythematosus, DeQuervains thyroiditis, thyroiditis, autoimmune asthma, myasthenia gravis, scleroderma, chronic hepatitis, Addison's disease, hypogonadism, pernicious anemia, vitiligo, alopecia areata, Coeliac disease, autoimmune enteropathy syndrome, idiopathic thrombocytopenic purpura, acquired splenic atrophy, idiopathic diabetes insipidus, infertility due to antispermatazoan antibodies, sudden hearing loss, sensoneural hearing loss, Sjogren's syndrome, polymyositis, autoimmune demyelinating diseases such as multiple sclerosis, transverse myelitis, ataxic sclerosis, pemphigus, progesssive systemic sclerosis, dermatomyositis, polyarteritis, nodosa, hemolytic anemia, glomerular nephritis and idiopathic facial paralysis. Diseases wherein the autoimmune response is a component of the host defense mechanism and disease process are also relevant to this invention. These include, but are not limited to, athero sclerotic plaque development, transplant rejection, host vs. graft disease, and others yet to be described.

### BRIEF DESCRIPTION OF THE DRAWINGS

FIGS. 1A-1D show deoxyribonucleic acid (DNA) induces MHC expression in cells.

FIGS. 2A-2B show properties of the nucleic acid generally needed to induce MHC expression in cells.

FIG. 3 shows the effects of KIFN and transfection with double-stranded deoxyribonucleic acid (dsDNA) or double-stranded ribonucleic acid (dsRNA) on genes responsible for antigen presentation.

FIGS. 4A-4C show dsDNA activates STAT 1 and 3, MAPK, and NF-PB.

FIGS. 5A-5B show the effects of dsDNA and KIFN are additive or, possibly synergistic; and tissue damage by electrical pulsing increases MHC expression coordinately with the release of genomic DNA into the cytoplasm.

FIG. 6 shows a drug is able to suppress the increase in expression of genes for MHC and antigen presenting molecules induced by double strand polynucleotides.

FIG. 7 shows the bovine TSH-induced cAMP response of hTSHR-transfected fibroblasts.

FIG. 8 shows the surface Expression of MHC Class II (Column 2) and Class I (Column 3) molecules on the surface of murine fibroblasts induced by double strand poly nucleotides and used for immunization in Table 1 and FIGS. 9-11.

FIG. 9 shows the effect of transfecting 5 Tg dsDNA into hTSHR DAP.3 cells used for immunization in Table 1 and FIGS. 9-11; the effect on genes responsible for antigen presentation is measured.

FIG. 10 shows the thyroids of mice immunized with hTSHR-DAP.3 cells transfected with dsDNA (A, B) or subjected to a sham tranfection procedure with lipofectamine alone (C, D). Thyroid glands were fixed in formalin for histological examination after hematoxylin-eosin staining. Magnification is same for B and D.

FIG. 11 shows the ability of IgG from hyperthyroid mice immunized with DNA-transfected hTSHR DAP.3 cells to increase cAMP levels, i.e., their stimulating TSHRAb activity. The data presented were obtained from one mouse but were duplicated in all hyperthyroid mice in Table 1.

FIG. 12 shows nucleotide sequence (SEQ ID NO:19) and predicted amino acid sequence (SEQ ID NO:20) of the rat 90K tumor-associated immunostimulator. The putative signal peptide is indicated by a bracket. The SRCR homology domain is boxed. Cysteine residues are underlined. Potential asparagine-linked glycosylation sites are circled.

FIG. 13 shows the comparison of the human (SEQ ID NO:21), rat (SEQ ID NO:22) and mouse (MAMA) (SEQ ID NO:23) homologs of the 90K tumor-associated immunostimulator. Amino acid identities in all three homologs are boxed; an identity of the rat 90K protein sequence with one other homolog is denoted by a dot. Nonidentical but similar residues are in white in the black boxes.

FIG. 14 shows the ability of dsDNA, KIFN, or both to increase 90K RNA levels relative to MHC Class I or Class II levels. Northern analyses were performed after 48 hours.

FIG. 15 show the ability of different polynucleotide examples of dsDNA, dsRNA, or single strand DNA or RNA to increase 90K RNA levels relative to MHC Class I or Class II levels. Northern analyses were performed after 48 hours.

FIG. 16 shows the ability of CpG oligonucleotide (A) vs viral or eukaryote dsDNA (B) to increase 90K RNA levels. Northern analyses were performed after 48 hours. Single-stranded CpG oligonucleotide are those described (D. M. Kliunman, et al., Proc. Natl. Acad. Sci. U.S.A. 93: 2879-2883 (1996) and FIG. 2a. The HSV2 and salmon sperm DNA were those used in FIG. 1a and 1b.

FIG. 17 shows the ability of different polynucleotides to increase 90K RNA levels as a function of concentration (A), length (B), or structure (C and D). Northern analyses were performed after 48 hours.

FIG. 18 shows the ability of a pRcCMV to modulate rat 90K and MHC Class I RNA levels when transfected into FRTL-5 cells maintained 6 days in 5H/5% serum (no TSH) or in 6H/5% serum (plus TSH) before transfection. Northern analyses was performed after 48 hours.

FIG. 19 shows the ability of dsDNA to bind to 90K protein measured by displacement chromatography on Sephadex G-100. In A, the radiolabeled DNA or 90K recombinant protein are run separately (−) or after incubation with each other (+). In B, the experiment was performed with an excess of unlabeled dsDNA oligonucleotide, poly(dI-dC) as a competitor. In (C), the radiolabeled DNA or crystalline bovine albumin are run separately (−) or after incubation with each other (+).

FIG. 20 shows the ability of ds nucleic acids to antagonize S-phase arrest induced by methimazole in FRTL-5 rat thyroid cells. Analyses were 36 hours after treatments.

FIG. 21 shows the effect of compound 10 and ds nucleic acids on the cell cycle in FRTL-5 rat thyroid cells. Analyses were 36 hours after treatments.

FIG. 22 shows a model of the development of autoimmune diseases and the effects of methimazole or tautomeric cyclic thiones on the development process.

### DESCRIPTION OF SPECIFIC EMBODIMENTS

For the purpose of a more complete understanding of various aspects or embodiments of this invention, the following definitions, descriptions, and examples are included.

Organisms that would benefit from this invention are those with an immune system capable of activating immune recognition molecules by the processes described. Such organisms may include primates, rodents, companion or farm animals, fish, and amphibians; in particular, humans, monkeys, mice, rats, hamsters, rabbits, dogs, cats, birds, cows, pigs, horses, sheep, and goats. By treatment of a disease or other pathological condition in an organism, we mean preventing the disease or condition, slowing disease progression or pathogenesis, reducing the occurrence and/or severity of a symptom, inducing and/or extending remission, increasing the organism's quality of life, or combinations thereof.

Major histocompatibility complex (MHC) is a generic designation meant to encompass the histocompatibility systems described in different species, including the human HLA, swine SLA, and mouse H-2 systems. Knowledge of the genetic organization and molecular biology of the MHC allow manipulation and identification of the encoded molecules. Increases in Class I and Class II are evident in 100% of cells transfected with 1 to 20 Tg ds nucleic acids/2×10⁶ cells. The effect is evident within 12 hrs and persists at least for 72 hours. Higher concentrations have greater effects on RNA levels of MHC or antigen presenting genes but maximize at about 5 Tg.

A polynucleotide is a polymer of ribonucleosides, deoxyribonucleosides, pyrimidine derivatives, purine derivatives, derivatives with a modified base, derivatives with a modified pentose sugar, and combinations thereof. Linkages may comprise phosphate, sulfur, and/or nitrogen atoms. The double-stranded polynucleotide used in this invention must have a sufficient length of duplexed strands to activate immune recognition molecules; this would not exclude the possibility that there are other regions of the polynucleotide that are, for example, single stranded, conjugated, or complexed to other chemical groups. Enzymatic synthesis is preferred for nonnatural polynucleotides such as DNA and RNA, but chemical synthesis without use of enzymes is preferred for nonnatural polynucleotides. The length of duplex strands sufficient for activity in this invention may be determined using the objectives and descriptions provided herein but a preferred length is at least about 25 base pairs (bp). Shorter ds polynucleotides, 25 to 35 bp require higher concentrations, at least about 10 to 50 Tg to elicit good responses; above 50 bp, generally 5 Tg or less elicits a maximal response.

Chemical and physical processes may be used for transfection (e.g., calcium phosphate precipitation, cationic lipid, DEAE-dextran, electroporation, microinjection). Alternatively, introduction of double-stranded polynucleotide may occur by intracellular entry by an infectious agent (e.g., bacterium, protozoan, virus), phagocytosis of a cell or infectious agent, replication of a single-stranded virus, oncogenic transformation, or an exogenous or environmental stimulus. In the latter instance, injury to the cell may cause leakage of DNA from the nucleus and/or mitochondria into the cytoplasm.

Tissue includes single cells, cells, whole organs and portions thereof, and may be comprised of a mixed or single population (e.g., epithelial, endothelial, mesenchymal, parenchymal cell types). Tissues may be recognized by their anatomical organization or biological function. In particular, tissue-specific antibody and histochemistry are useful in distinguishing different tissue types, assaying expression of tissue-specific function, and determining activation state of a tissue.

Tissue types which may be induced to activate immune activation molecules include but are not limited to muscle cells, endothelial cells, fibroblasts, and endocrine cells, i.e., thyrocytes, pancreatic islet cells and anterior pituitary cells. Some immune cells which may be used are lymphocytes, macrophages, dendritic cells; these are distinguished from the cells above by their expression of the MHC Class II gene, which is not detectable on normal, nonprofessional antigen presenting cells prior to activation. In vitro culture may be accomplished in organ perfusion, as a slice, or with dispersed cells on a substrate or in suspension. Culturing conditions which preserve the function or differentiated state of the tissue are preferred.

A drug is any chemical that shows activity in this invention. The drug may be a natural product found in animals, bacteria, fungi, molds, protozoa, or plants; artificially synthesized by chemical reactions from simple compounds or more complicated precursors; recombinantly synthesized by abzymes, enzymes, other engineered catalysts, transformed cells, or transgenic organisms; or combinations thereof. For example, active in this invention, with or without a pharmaceutically-acceptable carrier, are methimazole, methimazole derivatives, thione, thione derivatives, or pharmaceutical compositions comprising a safe and effective amount of a compound selected from:

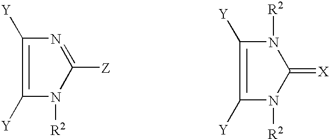

Wherein Y is selected from the group consisting of H, C₁-C₄ alkyl, C1,-C₄ substituted alkyl, —NO₂, and the phenyl moiety: 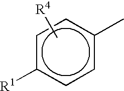
and wherein no more than one Y group in said active compound may be the phenyl moiety; R¹ is selected from the group consisting of H, —OH, C₁-C₄ alkyl, and C₁-C₄ substituted alkyl; R² is selected from the group consisting of H, C₁-C₄ alkyl, and C₁-C₄ substituted alkyl; R³ is selected from the group consisting of H, C₁-C₄ alkyl, C₁-C₄ substituted alkyl and —CH₂Ph; R⁴ is selected from the group consisting of H, C₁-C₄ alkyl, and C₁-C₄ substituted alkyl; X is detected from S and O; and Z is selected from —SR³, —OR³ and C₁-C₄ alkyl; and wherein at least two of the R² and R³ groups in said compound are C₁-C₄ alkyl when Y is not a phenyl moiety, and at least one Y is —NO₂ when Z is alkyl. These same drugs can be used to prevent the autoimmune response of a viral or bacterial infection, tissue damage such as that caused by atherosclerotic plaque development, and transplantation rejection.

Drugs may also be isolated from the foreign or endogenous substances active in this invention. Such substances may originate from infection, the surrounding environment, or the organism itself and induce, prevent, or suppress activation of immune recognition molecules. Double-stranded polynucleotide is an example of an active substance that induces activation; this substance may be introduced into a cell by a pathogen (e.g., bacterium, fungus, mold, protozoan, virus), transfection, leakage of genetic material from the nucleus or mitochondria, or other damage to cells of the organism. Substances that induce, prevent, or suppress activation of immune recognition molecules may be identified by measuring their effect on activation. For example, a biological sample (e.g., lysed cell or pathogen, tissue extract, blood, cerebrospinal fluid, lymph, lavage or fraction thereof) may be mixed with a cell before, after, or at about the same time as activation of MHC expression on the cell. If the biological sample prepared with and without infection by a pathogen differed in its effect on activation of MHC expression, it may indicate that a substance produced by the pathogen (i.e., foreign) or in response by the infected cell (i.e., endogenous) is present in the biological sample.

The drug may be formulated as a purified compound or a composition. For example, compounds not active in this invention may be added to the composition for ease of manufacture, storage, and/or transportation; stabilization of its chemical and/or physical properties; improved bioavailability, delivery, metabolism, and/or other pharmaceutically desirable properties of the drug; or combinations thereof. Suitable vehicles may be buffered to physiological pH and ionic strength; polar or nonpolar vehicles may be used to solubilize the formulation. Drugs may be combined for additive or synergistic effect.

By a drug or substance capable of enhancing or suppressing expression of an immune recognition molecules, we mean a drug or substance that has the ability to affect (increase or decrease) activation of immune recognition molecules on a cell or in an organism treated with the drug or substance relative to non-treated cell or organism before, at about the same time as, or after introduction of double-stranded polynucleotide. Selection of a drug or substance by its in vitro activity in this invention may then lead to assaying its in vivo activity in an animal model, which is preferably a model for a human disease or other pathological condition. These models include, but are not limited to, the 16/6 Id SLE model, the (NZBxNZW)F₁ mouse SLE model, the NOD mouse model and models of experimental blepharitis or uveitis (D. S. Singer, U.S. Pat. No. 5,556,754 issued Sep. 17, 1996; L. D. Kohn, et al., Methimazole derivatives and tautomeric cyclic thiones to treat autoimmune disease. U.S. Patent application filed Aug. 31, 1998)).

Administering a drug or substance capable of enhancing activation of immune recognition molecules may be used to develop an animal model of autoimmunity; targeting the drug or substance to a specific tissue may cause tissue-specific autoimmunity. In particular, this invention relates to processes for administering to an organism in need of such treatment a drug or substance capable of suppressing activation of immune recognition molecules, and may be used to treat a disease or other pathological condition (e.g., autoimmunity).

An effective dose of the drug or substance for administration may be determined using the objectives and description of the invention as disclosed herein. The drug or substance may be administered as a bolus at an interval determined by the organism's metabolism, or as divided doses that may maintain a selected concentration in the organism. Factors that may influence the amount of the effective dose are the disease or condition to be treated; age, family background, health, medical history, metabolic status, and/or sex of the organism to be treated; interactions with other medical and/or surgical treatment of the organism; and combinations thereof. In specific instances, treatment regimens or protocols for an organism would be at the discretion of a physician or veterinarian.

Although purified compounds are preferred for some purposes, drugs include extracts, powders, solutions, and other crude mixtures from which more purified compounds can be isolated by known processes (e.g., centrifugation, chromatographic or electrophoretic techniques, specific binding to affinity receptors or ligands) using this invention as an assay to determine enrichment of the activity. For example, a crude mixture may show activity in this invention and be separated according to the properties of its components into individual fractions. Each fraction can be assayed by this invention to identify those fractions which contain active components. Enrichment would result if the specific activity (e.g., activity normalized for mass of solute or volume of solvent) increased after separation, although interpretation of results may be complicated because more than one component is active or individual components are acting synergistically. Determining the activity in each fraction, comparing the total activity before and after separation, and constructing a balance sheet of activity with respect to the mass of material and its volume may show inter alia whether the presence of certain chemical structures in the fractions correlated with the activity, the existence of different components that are active, components that non-specifically increase or decrease activity in a fraction, the additive or synergistic nature of components, and if the particular isolation process used for separation was responsible for any reduction in activity. Synergy would be indicated if mixing fractions resulted in greater activity than would be predicted from the additive effect of the individual fractions; such mixing of fractions would also indicate whether there were non-specific activators or inhibitors of the assay (i.e., activators or inhibitors that did not specifically interact with an active component of the crude mixture) present in a fraction.

In drug screening programs, natural product or combinatorial libraries may be used to identify lead compounds and/or to select derivatives that are structurally related but functionally improved. Pharmaceutical products may be found to be active in this invention, derivatives of those products may be made, and derivatives may be selected according to the criterion that they have retained or improved functions. These functions may be activity in this invention, reduced side effects in an organism, or other pharmaceutically desirable activities as described above.

To facilitate purification and/or screening, processes may be automated and/or miniaturized, samples may be manipulated by robotics, reactants and/or their products may be immobilized, reactions may be arranged in fixed or variable spatial relationship to each other, or combinations thereof. For drug screening, a high-throughput system that quickly processes a large number of samples is preferred. For example, a high throughput system using cells stably transfected with MHC promoter elements may be used (L. D. Kohn, et al., Methimazole derivatives and tautomeric cyclic thiones to treat autoimmune disease. U.S. patent application filed Aug. 31, 1998)). Preferably, a combinatorial library of structurally related drugs may be immobilized on a solid substrate (e.g., derivatization of a core chemical structure with photoactivatable groups and/or photolabile linkages attached to a silicon wafer as a microarray) or duplicated from a master template (e.g., arranging different chemical structures in separate wells of a 96-well plate, dividing the solution in each well, depositing the divided solution into a reference plate and an arbitrary number of test plates, the locations of the wells of reference and test plates being in register and each well in register containing the same chemical structure). Other examples are immobilizing or cryopreserving cells on a solid substrate, contacting the immobilized cells with different drugs at predetermined locations on the solid substrate and identifying drugs by activation of immune recognition molecules on cells immobilized at only certain locations on the solid substrate. Alternatively, cells may be immobilized or cryopreserved in separate wells of a plate, cells can be exposed to different drugs in each well, and drugs can be identified by activation of immune recognition molecules on cells in certain wells of the plate.

Activation of an immune recognition molecule may be measured directly or by bioassay. Transcription of the immune recognition gene may be determined from promoter activity or abundance of RNA transcripts; translation of the immune recognition protein may be determined by metabolic labeling or abundance at the cell surface. Transcription, post-transcriptional processing, translation, and post-translation processing are all steps at which expression of the immune recognition molecule may be regulated. Moreover, the biological functions of the immune recognition molecule may be determined in a bioassay. Measurements of expression may be qualitative, semi-quantitative, or quantitative.

A simple example of a bioassay is measuring the immunogenicity of a cell activated by this invention when introduced into an organism. The activated antigen presenting cell (APC) may be a allogeneic or xenogeneic target depending on the genetic relationship between the activated APC and the organism, or a syngeneic target may present antigen in an MHC-restricted manner to the immune system of the organism. In the latter example, the immune system may be sensitized or tolerized to the antigen-MHC complex presented by the activated APC. The immune response in the organism can be measured, for example, by chromium release for T cell killing, cytokine release or plaque formation for T cell help, and footpad swelling for delayed-type hypersensitivity.

Specific binding assays may be used to detect immune recognition molecules: for example, antibody-antigen, receptor-ligand, and hybridization between complementary polynucleotides. The format of the assay may be direct or indirect, competitive, heterogeneous or homogeneous, amplified, or combinations thereof. Particular assays that may be used are immunoassay (e.g., RIA), cell sorting and analysis (e.g., FACS), nucleic acid amplification (e.g., PCR), nuclease protection, Western and Northern blots, and other known in the art.

Conveniently detected labels for use in this invention are radioisotopes, spin resonance labels, chromophores, fluorophores, and chemiluminescent labels. Optical detection systems and signal amplification are preferred. Thus scintillators may be used with radioisotopes or enzymes (e.g., horseradish peroxidase, alkaline phosphatase, luciferases and other fluorescent proteins) may be used for increased sensitivity.

Conjugation chemistry and fusion polypeptides made by recombinant technology can also be used to advantage. Non-covalent interactions, such as biotin-avidin and digoxygenin-antibody; covalent interactions formed by chemical crosslinkers or ligase; and fusion polypeptides may be used for immobilization or combining different functions into a single structure. For example, the microarrays described above may be arranged by immobilizing different elements at predetermined locations by photolithography using photoactivatable crosslinkers. A biosensor may be made by ligating the promoter of the gene encoding an immune recognition molecule to a marker gene, inducing activation by this invention may direct transcription of the marker gene, and determining expression of the marker may be more convenient than a similar determination of expression of the immune recognition molecule. For example, using green fluorescent protein (GFP) as the marker in a transcriptional fusion with a promoter for an MHC gene may allow measurement of the MHC gene's transcription, or localizing a pH-sensitive GFP derivative to secretory vesicles by a translational fusion with an MHC protein fragment may allow measurement of the MHC protein's appearance on the cell surface. Measurements with a biosensor would need to correlate with the cell's activation of the immune recognition molecule.

Examples of autoimmune conditions or diseases that can be treated by this process include, but are not limited to, rheumatoid arthritis, psoriasis, juvenile diabetes, primary idiopathic myxedema, systemic lupus erythematosus, De Quervains thyroiditis, thyroiditis, autoimmune asthma, myasthenia gravis, scleroderma, chronic hepatitis, Addison's disease, hypogonadism, pernicious anemia, vitiligo, alopecia areata, celiac disease, autoimmune enteropathy syndrome, idiopathic thrombocytopenic purpura, acquired splenic atrophy, idiopathic diabetes insipidus, infertility due to antispermatazoan antibodies, sudden hearing loss, sensoneural hearing loss, Sjogren's syndrome, polymyositis, autoimmune demyelinating diseases such as multiple sclerosis, transverse myelitis, ataxic sclerosis, pemphigus, progressive systemic sclerosis, dermatomyositis, polyarteritis nodosa, chronic hepatitis, hemolytic anemia, progressive systemic sclerosis, glomerular nephritis and idiopathic facial paralysis. Examples of diseases wherein the autoimmune response is a component of the host defense mechanism and disease process include but are not limited to altherocleotic plaque development, transplant rejection, and host vs graft disease. Autoimmune disease includes, but is not limited to, autoimmune dysfinctions and autoimmune disorders. Animal models include, but are not limited to, the 16/6 Id SLE model, the (NZBxNZW) F₁ mouse SLE model, the NOD mouse model and models of experimental blepharitis or uveitis (D. S. Singer, U.S. Pat. No. 5,556,754 issued Sep. 17, 1996; L. D. Kohn, et al., Methimazole Derivatives and tautomeric cyclic thiones to treat autoimmune disease. U.S. patent application filed Aug. 31, 1998)).

Abnormal or aberrant expression of major histocompatibility (MHC) Class I and Class II molecules in various tissues is associated with autoimmune reactions. We show that any fragment of double-stranded naked DNA or RNA, not only viral DNA, introduced into the cytoplasm of non-immune cells, causes abnormal MHC expression and the expression of other genes necessary for antigen presentation. The effect is not duplicated by single-stranded (ss) nucleic acids and is sequence-independent. The mechanism is distinct from and additive to that of KIFN. Class I is increased more than Class II; KIFN increases Class II more than Class I. KIFN action is mediated by the Class II transactivator (CIITA); DNA does not similarly induce CIITA. Rather the DNA effect appears to be mediated by activation of STAT1, STAT3, MAPK and NF-PB, as well as by induction of RFX5 and IRF-1. dsRNA mimics dsDNA, but unlike dsDNA induces [symbol] IFN gene expression by the target cell. Tissue damage appears to mimic the dsDNA effect. Double-stranded polynucleotides introduced into the cytoplasm may, therefore, convert cells to antigen presenting cells; the results disclosed herein provide a mechanistic explanation for the association between events that generate cytoplasmic dsDNA (e.g., viral infection, tissue damage, onsgene transformats) and an autoimmune response.

### EXAMPLES

Of general interest are the disclosures of U.S. Pat. Nos. 4,608,341; 4,609,622; and 5,556,754 which are incorporated by reference herein. Many chemical, genetic, immunological, and other techniques that may be used with this invention are known;

general techniques are also described in books, handbooks, and manuals available from publishers such as, for example, Academic Press and Cold Spring Harbor Laboratory Press.

### Example 1:

VIRUS INFECTION OF MAMMALIAN CELLS INCREASES MHC GENE EXPRESSION DIFFERENTLY FROM KIFN; THE VIRUS CAN BE REPLACED BY ANY DOUBLE STRAND VIRAL, BACTERIAL, OR MAMMALIAN DNA

The development of organ- or tissue-specific autoimmune diseases is associated with abnormal expression of major histocompatibility (MHC) class I and aberrant expression of MHC class II antigens on the surface of cells in the target organ or tissue (G. F. Bottazzo, et al., Lancet 2: 1115-1119 (1983); I. Todd, et al., Annals. N.Y. Acad. Sci. 475: 241-249 (1986); J. Guardiola & A. Maffei, Crit. Rev. Immunol. 13:

247-268 (1993); D. S. Singer, et al., Crit. Rev. Immunol. 17: 463-468 (1997)). Abnormal expression of MHC molecules on these nonimmune cells can cause them to mimic antigen presenting cells and present self-antigens to T cells in the normal immune cell repertoire (M. Londei, et al., Nature 312: 639-641 (1984); N. Shimojo, et al., Proc. Natl. Acad. Sci. U.S.A. 93: 11074-11079 (1996)). This leads to a loss in self tolerance and the development of autoimmunity (G. F. Bottazzo, et al., Lancet 2: 1115-1119 (1983); I. Todd, et al., Annals. N.Y. Acad. Sci. 475: 241-249 (1986); J. Guardiola & A. Maffei, Crit. Rev. Immunol. 13: 247-268 (1993); D. S. Singer, et al., Crit. Rev. Immunol. 17: 463-468 (1997); M. Londei, et al., Nature 312 :639-641 (1984); N. Shimojo, et al., Proc. Natl. Acad. Sci. U.S.A. 93: 11074-11079 (1996)). There is no comprehensive explanation as to how abnormal MHC expression might develop in the target tissue or how this might contribute to the ensuing immune cell responses involved in autoimmunity.

Viral infections can ablate self tolerance, mimic immune responses to self antigens, and induce autoimmune disease (J. Guardiola & A. Maffei, Crit. Rev. Immunol. 13: 247-268 (1993); R. Gianani & N. Sarvetnick, Proc. Natl. Acad. Sci. U.S.A. 93: 2257-2259 (1996); M. S. Horowitz, et al., Nature Medicine 4: 781-785 (1998); H. Wekerle, Nature Medicine 4: 770-771 (

C. Benoist & D. Mathis, Nature 394: 227-228 (1998)). Recent work (M. S. Horowitz, et al., Nature Medicine 4: 781-785 (1998); H. Wekerle, Nature Medicine 4: 770-771 (1998); C. Benoist & D. Mathis, Nature 394: 227-228 (1998)); has suggested that viral triggering of diverse autoimmune diseases including rheumatoid arthritis, insulin-dependent diabetes, and multiple sclerosis is caused by local viral infection of the tissue not molecular mimicry. It is suggested this involves MHC genes, results in presentation of self-antigens, and induces bystander activation of the T cells. The mechanism for this is obscure, as is its relation to the immune cell cytokine/IFN response (M. S. Horowitz, et al., Nature Medicine 4: 781-785 (H. Wekerle, Nature Medicine 4: 770-771 (1998); C. Benoist & D. Mathis, Nature 394: 227-228 (1998)).

KIFN can certainly increase MHC gene expression in the target tissue (J. P-Y. Ting & A. S. Baldwin, Curr.Opin. Immunol. 5: 8-16 (1993)); however, the mechanism by which a tissue or target cell viral infection recruits and activates immune cells to produce KIFN is unclear. Additionally, it is unlikely that KIFN alone causes autoimmunity, since its administration does not induce typical autoimmune disease (F. Schuppert, et al., Thyroid 7: 837-842 (1997)). Moreover, generalized KIFN production by immune cells cannot account for cell-specific autoimmunity, i.e. destruction of pancreatic [symbol] but not I cells in insulin-dependent diabetes mellitus or involvement of only thyroid follicular not parafollicular C cells in autoimmune Graves' disease (G. F. Bottazzo, et al., Lancet 2: 1115-1119 (1983); I. Todd, et al., Annals N.Y. Acad. Sci. 475: 241-249 (1986); N. Shimojo, et al., Proc. Natl. Acad. Sci. U.S.A. 93: 11074-11079 (1996); A. K. Foulis, et al., Diabetologia 30: 333-343 (1987)).

It has long been noted that specific viruses or viral promoters linked to DNA inserts could increase MHC class I gene expression in cells in culture (D. S. Singer & J. E. Maguire, Crit. Rev. Immunol. 10: 235-257 (1990)). We wondered whether this might be related to a primary action of the virus on the target tissue to increase class I and how this might trigger the cascade of events leading to an autoimmune response.

These experiments were, therefore, performed to evaluate the effect of viruses and viral DNA on MHC expression. We used rat thyrocytes as a model; but validated the results in a multiplicity of cells.

Experimental Protocol

Cells—Rat FRTL-5 thyroid cells were a fresh subclone (F1) with all properties described (F. S. Ambesi-Impiombato, U.S. Pat. No. 4,608,341 (1986); L. D. Kohn, et al., U.S. Pat. No. 4,609,622 (1986); L. D. Kohn, et al., Methimazole derivatives and tautomeric cyclic thiones to treat autoimmune disease. U.S. Patent application submitted Aug. 31, 1998; D. S. Singer., et al., U.S. Pat. No. 5,556,754, issued Feb. 17, 1996; P. L. Balducci-Silano, et al., Endocrinology 139: 2300-2313 (1998); V. Montani, et al., Endocrinology 139: 290-302 (1998); M. Saji, et al., J. Biol. Chem. 272: 20096-20107 (1997)). They were grown in 6H medium consisting of Coon's modified F12 medium, 5% heat-treated, mycoplasma-free, calf serum, 1 mM nonessential amino acids, and a six hormone mixture: bovine TSH (1×10⁻¹⁰M), insulin (10 Tg/ml), cortisol (0.4 ng/ml), transferrin (5 Tg/ml), glycyl-L-histidyl-L-lysine acetate (10 ng/ml), and somatostatin (10 ng/ml). Cells, were fed every 2-3 days and passaged every 7-10.

The following cells or cell lines were also used: a human hepatoblastoma cell line, HuH7; primary cultures of rat and human pancreatic islet cells, primary and continuous cultures of human and mouse fibroblasts; NIH 3T3 cells; the Pre B cell line, WEHI231; the macrophage line, P381D1; human muscle cells, SkMC; human endothelial cells, HUVEC; mouse smooth muscle cells, C2C12; C3H mouse derived myoblast cells; a C57B/6 spleen-derived immature dendritic cell clone; and primary cultures of mouse spleen dendritic cells, mouse peritoneal macrophages, and mouse spleen macrophages. The medium on each of these cell systems was changed every other day and cells were passaged every 4-6 days.

The human hepatoblastoma cell line, HuH7, NIH 3T3 cells (ATCC CRL-1658), and primary cultures of human or mouse fibroblasts were grown in Dulbecco's modified Eagle's medium (DMEM) with 10% fetal bovine serum (FBS) (T. Kohama et al., J. Biol. Chem. 273: 23722-23728 (1998)). Mouse smooth muscle cells, C2C12, and C3H mouse derived myoblast cell lines were also grown in high glucose DMEM containing 10% FBS (C. Domer, et al., J. Biol. Chem. 273: 20267-20275 (1998)). The Pre B cell line, WEHI231, and the macrophage line, P381D1, was maintained in RPMI 1640 medium supplemented with 10% FBS and 5×10⁻⁵M mercaptoethanol (S. Miyamoto, et al., Mol. Cell. Biol. 18: 19-29 (1998)). Human muscle cells, SkMC (Clonetic, San Diego, Calif.), were grown in Hams F10 with 20% FBS and 0.5% Chick Embryo extract (Gibco BRL, Gaithersburg, Md.) (J. M. Aschoff, et al., Analytical Biochemistry 219: 218-223 (1994)). Human endothelium HUVEC cells (Clonetic, San Diego, Calif.) were cultured in Endothelial cell Growth Media (Clonetic, San Diego, Calif.) supplemented with 2% FBS and several hormones as described (C. F. Bennett, et al., J. Immunol. 152: 3530-3540 (1994)). The C57B/6 spleen derived immature dendritic cell clone was maintained in 10% DMEM containing mouse GMCSF and fibloblast-derived growth factor. Primary cultures of mouse spleen dendritic cells, mouse peritoneal macrophage cells, and spleen macrophages were established from the BALB/c mouse and cultured in DMEM containing 10% FBS. Islet cells were obtained from rat and human pancreas samples by collagenase digestion as described (L. Invarardi, University of Miami, personal communication) and maintained in medium described by Hayden Coon and F. S. Ambesi Impiombato (personal communication).

C2C12 and C3H mouse derived myoblast cell lines were a kind gift from Dr. Edward Nelson (NCI, Frederick, Md.). Peritoneal exudate cells were prepared from BALB/c mice as follows. Forty mg of thioglycollate medium (FTG; Sigma) was injected intraperitonealy. Five days later peritoneal exudate cells were collected and resuspended in cold PBS. Erythrocytes were lysed with ACK lysing buffer, and the medium was then replaced with serum-free DMEM. After incubation at 37° C. for 3 hours the media was replaced with 10% fetal bovine serum containing complete media Twenty four hours later, these cells were used for transfection.

Single cell suspensions of spleen and lymph node cells were prepared from 6-10 week old female BALB/c mice. Mice were sacrificed by cervical dislocation, and the spleen, mesentery, and inguinal lymph nodes removed. Cells were treated with ACK lysing buffer to eliminate erythrocytes, washed with 5% FBS in RPMI, then resuspended in the same medium, 5×10⁶ cells per 10 cm diameter dish.

Transfection Methods—All procedures used 10 cm diameter dishes. For transfection with Lipofectamine Plus (GIBCO BRL, Gaithersburg, Md.), 5 Tg DNA was mixed with 30 T1 of Plus reagent and 750 T1 of serum-free medium, then incubated for 15 min at room temperature. A mixture of 30 T1 of Plus reagent and 750 T1 of serum-free medium was then prepared and mixed with the above DNA-containing mixture before being added to the cells as follows. Cells were washed with serum-free medium and the above mixture was added. Three hours later, medium was replaced with serum-containing, normal culture medium. Transfections with Lipofectamine (GIBCO BRL, Gaithersburg, Md.) used the same protocol without Plus reagent. DEAE dextran transfections used material from 5 Prime-3 Prime, Boulder, Colo. Five Tg of DNA, mixed with 250 T1 of DEAE dextran and 4.75 ml of serum-free medium, was added to cells which had been washed with Dulbecco's phosphate buffered saline (DPBS), pH 7.4. Cells were incubated for 1 hour in a CO₂ incubator at 37° C. After aspirating this medium, 2.5 ml of 10% dimethyl sulfoxide (DMSO) was added; and cells allowed to stand at room temperature for 3 min. Cells were washed with 10 ml of DPBS twice and 10 ml of culture medium was added. For electroporation, cells were suspended with different amounts of DNA in 0.8 ml of DPBS and were pulsed at 0.3 kV, using various capacitances and a Gene Pulser (Bio-Rad, Richmond Va.). They were then returned to the culture dish and cultured in growth medium.

Nucleic Acids—These included the following. The following polynucleotides were made by Pharmacia Biotech, Piscataway, N.J.: the DNA homopolymers, poly(dA), poly(dC), poly(dI), poly(dT); the DNA duplexes, poly(dI)/poly(dT), poly(dG)/poly(dC), poly(dI)/poly(dC); the DNA alternating copolymers, poly(dA-dT)/poly(dA-dT), poly(dI-dC)/poly(dI-dC), poly(dG-dC)/poly(dG-dC), poly(dA-dC)/poly(dG-dT); the RNA homopolymers, poly(A), poly(C), poly(G), poly(I); and the RNA duplex, poly(I)/poly(C). Sonicated salmon sperm DNA was from (Stratagene, La Jolla, Calif.). Bacterial DNA, calf thymus DNA, and transfer RNA were from Sigma (St. Louis, Mo.). Single strand RNA was generated by in vitro transcription. Total RNA was from FRTL-5 cells as was total MRNA, cDNA, and genomic DNA. cDNA was isolated as described (K. Suzuki, et al., Mol. Cell. Biol. 1998; in press); and genomic DNA was purified using a Wizard Genomic DNA purification Kit (Promega, Madison, Wis.). Viral DNA was from human herpes simplex virus; viral DNA oligonucleotides were from human immunodeficiency virus (HIV), human T lymphocyte virus (HTLV)-1, foamy virus, and cytomegalo virus (CMV). Plasmid vectors pcDNA3 and pRc/RSV, as well as their restriction fragments containing CMV promoter, SV40 promoter, ampicilin-resistant genes, neomycin resistant genes, multicloning sites, etc., were used with or without methylation or DNase-treatment. Plasmid DNAs were purified using EndoFree Plasmid Maxi Kits (QIAGEN, Valencia, Calif.). Single strand or double strand oligonucleotides were 25 bp to 54 bp in length. Single or double strand phosphorothioate oligonucleotides (s-oligos) were 54 bp.

Northern Analysis—Total RNA was prepared and Northern analysis performed for MHC class I, MHC class II, and glyceraldehyde phosphate dehydrogenase (GAPDH) as described (M. Saji, et al., J. Clin. Endocrinol. Metab. 75: 871-878 (1992); P. L. Balducci-Silano, et al., Endocrinology 139: 2300-2313 (1998); V. Montani, et al., Endocrinology 139: 290-302 (1998); S.-I. Taniguchi, et al., Mol. Endocrinol. 12: 19-33 (1998)). Probes for MHC class I and class II are those described (M. Saji, et al., J. Clin. Endocrinol. Metab. 75: 871-878 (1992); P. L. Balducci-Silano, et al., Endocrinology 139: 2300-2313 (1998); V. Montani, et al., Endocrinology 139: 290-302 (1998); S.-I. Taniguchi, et al., Mol. Endocrinol. 12: 19-33 (1998)). The glyceraldehyde phosphate dehydrogenase (GAPDH) probe used was cut from a pTR1-GAPDH-Rat template (Ambion, Tex.). The pTR1-GAPDH rat template was digested using restriction enzymes Sac I and BamHI to release a 316 bp fragment. The fragment was cut from agarose gels, purified using JetSorb Kit (PGC Science, Frederick, Md.), and subcloned into a pBluescript SK(+) vector at the same restriction site.

Flow Cytometry Analysis—FACS was performed by a modification of methods described (M. Saji, et al., Proc. Natl. Acad. Sci. U.S.A. 89: 1944-1948 (1992); T. F. Davies, et al., Clin. Endocrinol. 31: 125-135 (1989); N. Shimojo, et al., Proc. Natl. Acad. Sci. U.S.A. 93: 11074-11079 (1996); K.-I. Yamaguchi, et al., J. Clin. Endocrinol. Metab. 82: 4266-4269 (1997); S. Kikuoka, et al., Endocrinology 139: 1891-1898 (1998)). In brief, transfected cells were washed with cold PBS and harvested by scraping after incubation with 0.5 mM EDTA-PBS for 5 min. at room temperature. After these single cell suspensions were prepared and washed with phosphate buffered saline (PBS) at pH 7.4, 10⁶ cells were pelleted, suspended in 100 T1 PBS, and placed in individual wells of a 96-well flat-bottomed plate. One million cells were incubated with 0.2 Tg blocking antibody for 10 min. (except C2C12 cells). They were then treated for 30 min on ice with 100 T1 (0.5 Tg) of the various fluorescein-isothiocyanate (FITC)- or PE labeled antibodies labeled human, rat, or mouse specific monoclonal antibodies against MHC class I or class II antigens relevant to the species of cell used (Serotec, Raleigh, N.C.). Alternatively FITC-anti-mouse H-2Kb (mouse IgG2a), FITC-anti-mouse I-Ab(Aab) (mouse IgG2a), FITC-anti-mouse H-2Dd (mouse IgG2a), FITC-anti-mouse I-Ad/I-Ed (control:Rat IgG2a), FITC-anti-mouse H-2Dk (mouse IgG2a), FITC-anti-mouse I-Ek (mouse IgG2a) FITC-anti-mouse CD86(B7-2) (rat IgG2a), PE-anti-mouse CD11b (Mac-1), Cy-chrome-anti-mouse TCR beta chain (hamster IgG) were purchased from Pharmingen. Cells were washed three times, and kept in the dark at 4° C. until FACS analysis was performed. Optimal dilution of each antibody, i.e. a concentration which did not give non-specific binding of antibody to the cell surface was pre-determined. Leu-4 was used as a background control and a subclass-matched immunoglobulin fraction served as the negative control antibody (Becton Dickinson, Mountain View, Calif.) in each analysis. After being washed with 0.1% BSA-0.1% NaN₃-PBS, FACS analysis was performed using a FACSc an instrument and Cell Quest software (Becton Dickinson, San Jose, Calif.) (M. Saji, et al., Proc. Natl. Acad. Sci. U.S.A. 89: 1944-1948 (1992); T. F. Davies, et al., Clin. Endocrinol. 31: 125-135 (1989); N. Shimojo, et al., Proc. Natl. Acad. Sci. U.S.A. 93: 11074-11079 (1996); K.-I. Yamaguchi, et al., J. Clin. Endocrinol. Metab. 82: 4266-4269 (1997); S. Kikuoka, et al., Endocrinology 139: 1891-1898 (1998)).

Results

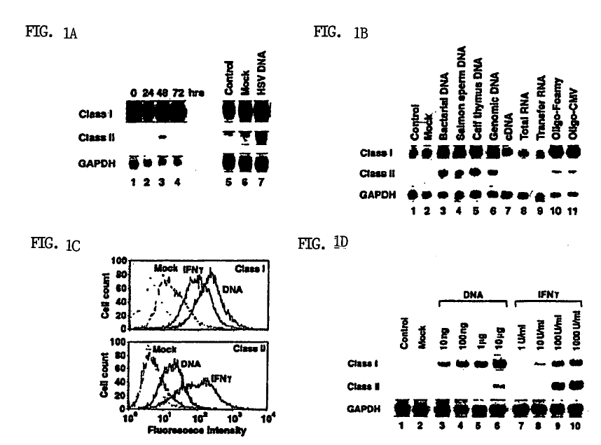

FRTL-5 cells were grown in 10 cm dishes (D. S. Singer & J. E. Maguire, CRC Crit. Rev. Immunol. 10: 235-257 (1990); S. I. Taniguchi, et al., Mol. Endocrinol. 12: 19-33 (1998); P. L. Balducci-Silano, et al., Endocrinology 139: 2300-2313 (1998); V. Montani, et al., Endocrinology 139: 290-302 (1998); K. Suzuki, et al., Endocrinology 139: 3014-3017 (1998); to a density of 2×10⁶ cells. In FIGS. 1A and 1B, FRTL-5 cells were infected with herpes simplex virus (HSV-2) as described (P. R. Krause, et al., J. Exp. Med. 181: 297-306 (1995)), (FIG. 1A, lanes 1-4). Alternatively, they were transfected with 5 Tg HSV DNA fragments (FIG. 1A, lane 7), other noted DNAs (FIG. 1B, lanes 3-7), RNA (FIG. 1B, lanes 8, 9) or 54 bp double-stranded oligodeoxynucleotides (ODNS) from Foamy or cytomegalovirus (FIG. 1B, lanes 10, 11) using the cationic lipid LIPOFECTAMINE PLUS (GIBCO BRL, Gaithersburg, Md.) and the manufacturer's protocol. Total RNA was prepared and Northern analysis performed for MHC Class I, MHC Class II, or glyceraldehyde phosphate dehydrogenase (GAPDH) as described (D. S. Singer & J. E. Maguire, CRC Crit. Rev. Immunol. 10: 235-257 (1990); Taniguchi, S. I. et al., Mol. Endocrinol. 12: 19-33 (1998); P. L. Balducci-Silano, et al., Endocrinology 139: 2300-2313 (1998); Suzuki, K. et al., Endocrinology 139: 3014-3017 (1998); and at either the times noted or 48 hours after treatment. Cationic lipid treatment alone served as a control of the transfection procedure (Mock). In FIG. 1C, FACS analysis of cell-surface Class I and Class II expression induced by DNA or 100 U/ml rat KIFN 48 hours after treatment.

Cells were transfected with 5 Tg pcDNA3 (Invitrogen, Calif.) exactly as for all dsDNAs in FIGS. 1A and 1B and as in Example 2. The dashed line represents control staining with FITC-labeled normal mouse IgG₁. In FIG. 1D, FRTL-5 cells were transfected with 10 ng to 10 Tg dsDNA (lanes 3-6) or were exposed to 1 to 1000 U/ml KIFN in the culture medium (lanes 7 to 10). RNA was prepared and Northern analysis performed 48 hrs after either treatment.

To study whether there is a direct effect of nucleic acids on MHC expression, we treated a model normal cell, rat FRTL-5 thyroid cells, with herpes simplex virus or transfected them with various viral and other DNA preparations, including DNA from foreign or self origin and ODNs from viral DNA sequences (FIG. 1). Rat FRTL-5 cells are a continuously cultured cell line derived from normal thyroids, which maintain normal thyroid function in vitro, and are a model system to study thyroid autoimmunity (D. S. Singer & J. E. Maguire, CRC Crit. Rev. Immunol. 10: 235-257 (1990); S. I. Taniguchi, et al., Mol. Endocrinol. 12: 19-33 (1998); Balducci-Silano, P. L. et al., Endocrinology 139: 2300-2313 (1998); V. Montani, et al., Endocrinology 139: 290-302 (1998)).

Transfection was with lipofectamine plus Herpes simplex infection increased MHC RNA levels in the FRTL-5 cells within 48 hours of infection (FIG. 1A, lanes 1 to 4). However, transfected HSV DNA (FIG. 1A, lanes 5-7) and all double-stranded (ds) DNAs tested, but not single-stranded (ss) DNA, also increased MHC RNA levels after 48 hours (FIG. 1B). As will be evident in Example 2, in studies of MHC class II transcript levels, the degree of activation was improved with stronger double strand structures and there was no sequence specific motif. There was no effect on RNA levels of glyceraldehyde phosphate dehydrogenase (GAPDH) (FIG. 1A and 1B) indicating a degree of specificity; and control transfections without DNA had no effect (FIG. 1A, lane 6; FIG. 1B, lane 2).

Different transfection procedures using cationic lipid (LIPOFECTAMINE), electroporation, and DEAE-dextran also did not alter the results. Also microinjection into the cytoplasm of cells duplicated these results, as measured in individual cells by immunostaining using specific antibodies to MHC class I and class II as described in whole tissues with autoimmune disease (G. F. Bottazzo, et al., Lancet 2: 1115-1119 (1983); I. Todd, et al., Annals. N.Y. Acad. Sci. 475: 241-249 (1986)). There was no correlation with transfection efficiency; thus, under conditions where 100% of cells exhibited increased MHC class I and class II antigen expression (FIG. 1C), transfection efficiency, measured by including 2 Tg pGreen Lantern-I (GIBCO, BRL, Gaithersburg, Md.) and counting green fluorescent proitein expression in cells, was only 10%. Thus, it appears that it is sufficient to introduce the ds nucleic acids into the cytoplasm to have increased MHC gene expression and all phenomena to be detailed in Example 2.

These results were not limited to rat FRTL-5 thyroid cells but were duplicated in a human hepatoblastoma cell line, HuH7, in primary cultures of rat and human pancreatic islet cells, in primary and continuous cultures of human and mouse fibroblasts, in NIH 3T3 cells, in SkMC human muscle cells, in HUVEC human endothelial cells, in C2C12 mouse smooth muscle cells, in C34 mouse myoblast cells, in C57B16 spleen-derived dendritic cells in the WEHI231 Pre B cell line, in the P381D1 macrophage line, and in primary cultures of mouse spleen dendritic cells, mouse peritoneal macrophages, and mouse spleen macrophages. In each case there was an increase in class I and class II RNA levels and in MHC antigen presentation measured by FACS analyses, albeit this was less dramatic in the immune cells where constitutively high levels of MHC class I, MHC class II, or both exist, e.g. C57 B16 denductic cells, the P381D 1 macrophage line, and in primary cultures of mouse spleen dendritic cells, mouse peritoneal macrophages, and mouse spleen macrophages.

In sum, the phenomenon was not cell specific. Further, the islet cells, liver cells, endothelial cells, fibroblasts, and muscle cells, as well as the thyrocytes, are cell types in tissues or organs where autoimmune disease is known to occur or be a part of the tissue damage process, e.g. diabetes, insulitis, hepatitis, atherosclerosis, Graves' disease, thyroiditis, psoriasis, systemic lupus and related collagen diseases, alopecia, and myositis, to name but a few. Moreover, the increases measured in lymphocytes, macrophages, and dendritic cells indicate immune cells can be directly and similarly effected by the virus or its ds nucleic acid. Finally the phenomenon is not restricted to normal cells such as the FRTL-5 cell line which is fully functional and under hormonal control, but is also evident in cells which have greater or lesser levels of a transformed phenotype. Thus, induction of MHC expression by naked double-stranded polynucleotide is a widespread phenomenon.

The effect of DNA transfection on MHC expression in FRTL-5 cells was different from that of KIFN, both with respect to cell surface expression (FIG. 1C) and RNA (FIG. 1D). The dsDNA increased Class I gene expression more than Class II, independent of the intrinsic concentration-dependence of each (FIG. 1C and 1D).

One possible explanation for the action of the DNA relates to the role of non-methylated CpG motifs (A. M. Krieg, et al., Nature 374: 546-548 (1995); A. K. Yi, et al., J. Immunol. 156: 558-564 (1996); D. M. Klinman, et al., Proc. Natl. Acad. Sci. U.S.A. 93: 2879-2883 (1996)). Non-methylated CpG motifs within bacterial and viral DNA sequences have been shown to activate immune cells by inducing various cytokines in lymphocytes and macrophages and to induce immunoglobulin secretion in B cells (A. M. Krieg, et al., Nature 374: 546-548 (1995); A. K. Yi, et al., J. Immunol. 156: 558-564 (1996); D. M. Klinman, et al., Proc, Natl. Acad. Sci. U.S.A. 93: 2879-2883 (1996)).

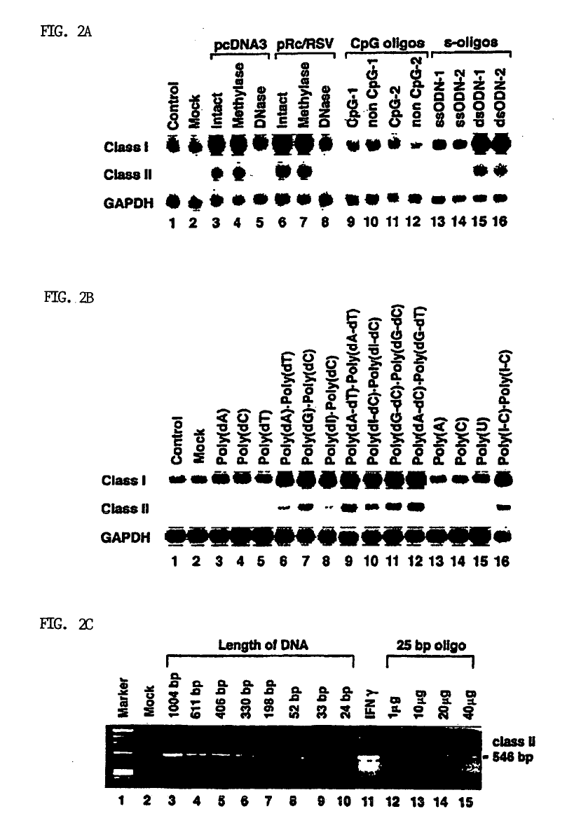

Transfection and Northern analysis were performed 48 hours after treatment, exactly as in FIG. 1. In FIG. 2A, FRTL-5 cells were transfected with intact, methylated or DNase-treated plasmid, pcDNA3 or pRc/RSV (Invitrogen, Calif.) (lanes 3-8), single-stranded CpG oligodeoxy nucleotides (ODNs) or control ODNs (lanes 9-12), or ss- or ds-phosphorothioate oligonucleotides (S-oligos) (lanes 13-16). Lane 1 contains RNA from non-treated cells and lane 2 from cells subjected to the transfection procedure only, i.e. without nucleic acids being present. In FIG. 2B, various synthetic polymer nucleotides and their duplexes (Pharmacia Biotech Inc., Piscataway, N.J.) were transfected and analyzed (lanes 3-16) as in FIG. 2A. In FIG. 2C, cells were transfected with 5 Tg of dsDNA fragments from 24 bp to 1004 bp in length (lanes 3-10) or with indicated amount of 25 bp dsODNs (lanes 12-15) as described above. In FIG. 2C, Class II expression was measured 48 hours later by RT-PCR as described previously (P. L. Balducci-Silano, et al., Endocrinology 139: 2300-2313 (1998); K. Suzuki, et al., Endocrinology 139: 3014-3017 (1998)). Cells treated with 100 U/ml KIFN for 48 hours were the positive control.

Although no evidence exists for direct CpG motif induction of MHC molecules in target cells, we evaluated the possible role of CpG motifs by transfecting FRTL-5 cells with intact or methylated dsDNA or known CpG oligodeoxynucleotides and their non-CpG controls (FIG. 2A). Both methylated and unmethylated plasmid DNA had similar effects on Class I and II induction (FIG. 2A, lanes 3 vs 4 and 5 vs 6). Also, neither the oligos having one or more CpG motifs (CpG-1; CpG-2), which were confirmed to induce interleukin 6, 12 or KIFN in lymphocytes (D. M. Klinman, et al., Proc. Natl. Acad. Sci. U.S.A. 93: 2879-2883 (1996)), nor their non-CpG controls, induced MHC expression (FIG. 2A, lanes 9 to 12) had different effects. The induction of MHC was, however, abolished when the DNA was pretreated with DNase (FIG. 2A, lanes 5 vs 3 and 8 vs 6), but not RNase (data not shown). Additionally, single-stranded phosphorothioate oligonucleotides (ss-S-oligos) had no effect, whereas ds-S-oligos induced MHC expression (FIG. 2A, lanes 13-16). The DNA effect on MHC expression therefore seems to be double-strand specific and not to involve CpG motifs.

To see if there is any sequence specificity, we transfected FRTL-5 cells with various synthetic polynucleotides (FIG. 2B). dsDNA copolymers (FIG. 2B, lanes 9-12) or duplexes (FIG. 2B, lanes 6-8) induced MHC expression, whereas ss polymers had no effect (FIG. 2B, lanes 3-5). Of interest, dsRNA, which is known to induce various anti-viral reactions, including induction of IFN, also induced MHC expression, whereas ssRNA had no effect (FIG. 2B, lanes 13-16). The DNA effect was length and concentration dependent (FIG. 2C); as short as 25 bp of double-stranded (ds) oligonucleotide was effective (FIG. 2C, lanes 12-15).

To summarize these results, activation of immune recognition molecules was sequence independent; short lengths of double-stranded polynucleotide were effective; and both dsRNA and dsDNA could be used. This last observation has relevance to the action of dsRNA intermediates formed during infections by RNA viruses and to the action of poly I-C, as will be shown below.

### Example 2:

ANY DOUBLE STRAND VIRAL, BACTERIAL, OR MAMMALIAN DNA NOT ONLY INCREASES MHC GENE EXPRESSION BUT ALSO INCREASES EXPRESSION AND ACTIVATION OF GENES IMPORTANT FOR ANTIGEN PRESENTATION AND THE GROWTH AND FUNCTION OF CELLS; THE ACTIONS ARE DIFFERENT FROM KIFN

To acquire antigen-presenting ability, a non-immune cell must coordinately activate or induce the expression of non-MHC genes and proteins important for antigen presentation (I. A. York & K. L. Rock, Annu. Rev. Immunol. 14: 369-396 (1996); J. Pieters. Curr. Opin. Immunol. 9: 89-96 (1997)). R. Ekholm, et al., Control of the thyroid gland: Regulation of its normal function and growth. Advances in Experimental Medicine and Biology, Vol. 261. Plenum Press, New York, pp. 1-403 (1989); L. D. Kohn, et aL, Intern. Rev. Immunol. 9: 135-165 (1992); L. D. Kohn, et al., Vitamins and Hormones, 50: 287-384 (1995); L. D. Kohn, et al., in Thyroid Immunity, D. Rayner and B. Champion (Eds.), R. G. Landes Biomedical Publishers, Texas, pp. 115-170 (1995); S. I. Taniguchi, et al., Mol. Endocrinol, 12: 19-33 (1998)). Changes in both must also be coordinated with the growth and function of cells. Changes in genes important for antigen presentation are required for the multiple steps involved in antigen processing and presentation. For example, increases in proteasome proteins (e.g., LMP2) and activity are necessary for antigen processing in Class I-restricted systems (I. A. York & K. L. Rock, Annu. Rev. Immunol. 14: 369-396 (1996)). Also, a transporter of antigen peptides (e.g., TAP-1, TAP-2) is required for the peptides to gain access to the secretory pathway, to bind the Class I molecule, and to form the antigen-MHC complex presented on the cell surface (I. A. York & K. L. Rock, Annu. Rev. Immunol. 14: 369-396 (1996)). In the case of Class II, invariant chain (Ii) and HLA-DM proteins are required to regulate binding of antigen peptides to MHC. Catabolism of antigen to peptide capable of binding Class I and/or Class II may occur by proteolysis in the cytoplasm or a specialized organelle (e.g., the lysosome). A co-stimulatory molecule (B7 molecules or CD80, for example) may also be needed to activate lymphocytes (J. Pieters, Curr. Opin. Immunol. 9: 89-96 (1997)).

The following experiments were, therefore, performed to evaluate the effect of ds polynucleotides on the expression or activation of genes important for antigen presentation as well as MHC expression. We again used rat thyrocytes as a model; but validated the results in a multiplicity of cells as described in example 1.

Experimental Protocol

Cells—Rat FRTL-5 thyroid cells were a fresh subclone (F1) with all properties described (F. S. Ambesi-Impiombato, U.S. Pat. No. 4,608,341 (1986); L. D. Kohn, et al., U.S. Pat. No. 4,609,622 (1986); L. D. Kohn, et al., Methimazole derivatives and tautomeric cyclic thiones to treat autoimmune disease. U.S. Patent application submitted Aug. 31, 1998; D. S. Singer., et al., U.S. Pat. No. 5,556.754, issued Feb. 17, 1996; P. L. Balducci-Silano, et al., Endocrinology 139: 2300-2313 (1998); V. Montani, et al., Endocrinology 139: 290-302 (1998); M. Saji, et al., J. Biol. Chem. 272: 20096-20107 (1997)). They were grown in 6H medium consisting of Coon's modified F12 medium, 5% heat-treated, mycoplasma-free, calf serum, 1 mM nonessential amino acids, and a six hormone mixture: bovine TSH (1×10⁻¹⁰M), insulin (10 Tg/ml), cortisol (0.4 ng/ml), transferrin (5 Tg/ml), glycyl-L-histidyl-L-lysine acetate (10 ng/ml), and somatostatin (10 ng/ml). Cells, were fed every 2-3 days and passaged every 7-10. In some experiments, cells were treated with 100 U/ml rat KIFN for the last 48 hours of culture.

The following cells or cell lines also used: a human hepatoblastoma cell line, HuH7; NIH 3T3 cells; the Pre B cell line, WEHI231; the macrophage line, P381 D1; human muscle cells, SkMC; human endothelial cells, HUVEC; mouse smooth muscle cells, C2C12; and primary cultures of mouse spleen dendritic cells. Methods for their growth are detailed in Example 1.

Transfection—All procedures used 10 cm dishes and transfection with Lipofectamine Plus (GIBCO BRL, Gaithersburg, Md.). As in Example 1, 5 Tg DNA was mixed with 30 T1 of Plus reagent and 750 T1 of serum-free medium, then incubated for 15 min at room temperature. A mixture of 30 T1 of Plus reagent and 750 T1 of serum-free medium was then prepared and mixed with the above DNA-containing mixture before cells were washed with serum-free medium and the above mixture added. Three hours later, medium was replaced with serum-containing, normal culture medium. Transfections with Lipofectamine (GIBCO BRL, Gaithersburg, Md.), with DEAE dextran, or using electroporation, performed as in Example 1, yielded the same results.

Nucleic Acids—The following polynucleotides were used in these experiments, both made by Pharmacia Biotech, Piscataway, N.J.: poly(dI)/poly(dC) and poly(I)/poly(C). The same results were obtained, however, using sonicated salmon sperm DNA (Stratagene, La Jolla, Calif.), bacterial DNA or calf thymus DNA (Sigma, St. Louis, Mo.), and FRTL-5 cell genomic DNA. Genomic DNA was purified using a Wizard Genomic DNA purification Kit (Promega, Madison, Wis.). Viral DNA from Human Herpes Simplex virus and viral DNA oligonucleotides from HIV, HTLV-1, Foamy virus, and cytomegalic virus (CMV) as well as the plasmid vectors pcDNA3 and pRc/RSV, used with or without methylation, also duplicated the results with the ds synthetic polynucleotides. Plasmid DNAs were purified using EndoFree Plasmid Maxi Kits (QIAGEN, Valencia, Calif.).

CpG oligonucleotides were those described (D. M. Klinman, et al., Proc. Natl. Acad. Sci. U.S.A 93:2879-83 (1996)). Methylation of CpG sites in plasmid DNA from pcDNA3, pRc/RSV, and their restriction fragments was by treatment with SssI methylase (New England BioLabs, Beverly, Mass.) at 37° C. for 2 hours. Methylation of CpG motif was confirmed by resistance to BstUI restriction enzyme (New England BioLabs) which recognizes 5′-CGCG-3′ motifs. For DNase I digestion, pcDNA3, pRc/RSV and their restriction fragments were treated with DNase I (Promega, Madison, Wis.) at 37° C. for 30 min, then extracted by phenol-chloroform followed by ethanol precipitation. Digestion was confirmed by agarose gel electrophoresis.

Northern Analysis—Total RNA was prepared and Northern analysis performed as described (M. Saji, et al., J. Clin. Endocrinol. Metab. 75: 871-878 (1992); P. L. Balducci-Silano, et al., Endocrinology 139: 2300-2313 (1998); V. Montani, et al., Endocrinology 139: 290-302 (1998); S.-I. Taniguchi, et al., Mol. Endocrinol. 12: 19-33 (1998)). Probes for MHC class I and class II are those described (M. Saji, et al., J. Clin. Endocrinol. Metab. 75: 871-878 (1992); P. L. Balducci-Silano, et al., Endocrinology 139: 2300-2313 (1998); V. Montani, et al., Endocrinology 139: 290-302 (1998); S.-I. Taniguchi, et al., Mol. Endocrinol. 12: 19-33 (1998)). The glyceraldehyde phosphate dehydrogenase (GAPDH) probe used was cut from a pTR1-GAPDH-Rat template (Ambion, Tex.). The pTR1-GAPDH rat template was digested using restriction enzymes Sac I and BamHI to release a 316 bp fragment. The fragment was cut from agarose gels, purified using JetSorb Kit (PGC Science, Frederick, Md.), and subcloned into a pBluescript SK(+) vector at the same restriction site. The probe for rat CIITA is a cloned rat Type III CIITA cDNA fragment in pcDNA3 (K. Suzuki et al., manuscript in preparation). EcoRI is used to release a 4098 bp fragment as the probe. The probe for rat 90 kDa Tumor-associated immunostimulator (A. Ullrich, et al., J. Biol. Chem. 269: 18401-18407 (1994)) is a cloned cDNA fragment described in Example 6. The probe for IRF-1 (GeneBank accession No. X14454) was cut from a plasmid kindly provided by Dr. T. Taniguchi, Osaka, Japan. It was cut from pUCIRF-1 which was kindly provided by Dr. Kenji Sugiyama, Nippon Boehringer Ingelheim Vo., Ltd, Hyogo, Japan. Hind III/BamHI was used to release a 2.1 kb fragment. Other probes were made by RT-PCR based on published cDNA sequences using following ODNs as primers: LMP2,

| | |
|---|---|
| TACCGTGAGGACTTGTTAGCG | (SEQ ID NO:1) |
| and | |
| ATGACTCGATGGTCCACACC (296 bp); | (SEQ ID NO:2) |
| TAP1, | |
| GGAACAGTCGCTTAGATGCC | (SEQ ID NO:3) |
| and | |
| CACTAATGGACTCGCACACG (504 bp); | (SEQ ID NO:4) |
| Invariant chain (Ii), | |
| AATTGCAACCGTGGAGTCC | (SEQ ID NO:5) |
| and | |
| AACACACACCAGCAGTAGCC (635 bp); | (SEQ ID NO:6) |
| HLA-DMB, | |
| ATCCTCAACAAGGAAGAAGGC | (SEQ ID NO:7) |
| and | |
| GTTCTTCATCCACACCACGG (222 bp); | (SEQ ID NO:8) |
| B7.1, | |
| CCATACACCGAATCTACTGGC | (SEQ ID NO:9) |
| and | |
| TTGACTGCATCAGATCCTGC (589 bp); | (SEQ ID NO:10) |
| RFX5, | |
| AAGCTGTATCTCTACCTTCAG | (SEQ ID NO:11) |
| and | |
| TTTCAGGATCCGCTCTGCCCA (470 bp); | (SEQ ID NO:12) |
| PKR, | |
| ACAAGGTGGATAGTCACACGG | (SEQ ID NO:13) |
| and | |
| CCAGATGCTGACTGAGAAGC (352 bp); | (SEQ ID NO:14) |
| θIFN, | |
| AAGATCATTCTCACTGCAGCC | (SEQ ID NO:15) |
| and | |
| TGAAGACTTCTGCTCGGACC (586 bp). | (SEQ ID NO:16) |

SDS-polyacrylamide gel electrophoresis and Western blotting—Transfected FRTL-5 cells or FRTL-5 cells treated with KIFN (100 U/ml protein) which had been grown in 100 mm dishes (Nalge Nunc International), were placed on ice before harvesting, washed with ice-cold Dulbecco's PBS (DPBS), released by gentle scraping with a rubber policeman, and collected by low-speed centrifugation at 833×g for 10 min in a Sorvall table-top centrifuge (rotor H-1000, Dupont Company, Wilmington Del.). After a second washing in DPBS, cells were resuspended in cold lysis buffer [50 mM HEPES pH 7.0,2 mM MgCl₂; 250 mM NaCl; 0.1 mM EDTA; 0.1 mM EGTA; 1 mM DTT; 2 mM Na₃VO₄; 10 mM Na₄P₂O₇; 10 mM NaF; 0.1% NP-40; 0.5 mM p-amidinophenyl methanesulfonyl fluoride hydrochloride (p-APMSF) plus a protease inhibitor cocktail (2.5 mg/ml of pepstatin A; 2.5 mg/ml of antipain; 2.5 mg/ml of chymostatin; 0.25 mg/ml leupeptin; 0.25 mg/ml antipain]. The cells were allowed to lyse on ice for 60 min, after which they were vortexed vigorously and centrifuged at 4° C. and at 12,000 rpm in a microcentrifuge for 10 min. The supernatant was collected and frozen in aliquots at −70° C. Before electrophoresis in sodium dodecyl sulfate (SDS) containing gels, cell lysates (50 Tg protein) were incubated with 62.5 mM Tris-HCl buffer pH 6.8 containing 2% SDS, 5% 2-mercaptoethanol, 7% glycerol and 0.01% bromophenol blue for 30 min at room temperature. SDS-gel electrophoresis was performed using 10 to 20% SDS Tris-Glycine gels as described (K. Laemmli, Nature 277: 680-685 (1970); T. Ban, et al., Endocrinology: 131: 815-829 (1992); A. Hirai, et al., J. Biol. Chem. 272: 13-16 (1997); Y. Noguchi, et al., J. Biol. Chem. 273: 3649-3653 (1998)); molecular weight markers were from NOVEX. After gel-electrophoresis, samples were transferred to nitrocellulose membranes by electroblotting at 30V for 2 hrs, as described (H. Towbin, et al., Proc. Natl. Acad. Sci. U.S.A. 76: 4350-4354 (1979)). Protein was identified after antibody binding using the ECL method (Amersham Life Science, Cleveland, Ohio) as described (A. Hirai, et al., J. Biol. Chem. 272: 13-16 (1997); Y. Noguchi, et al., J. Biol. Chem. 273: 3649-3653 (1998)). In brief, following blocking with a solution of 0.6% Tween 20, 10% skim milk, and 1% crystalline bovine serum albumin (BSA) overnight at room temperature, the buffer was replaced with a 1:500 dilution of primary antibody in blocking buffer which was diluted 1:10 in PBS-Tween. After incubation for 1 hour, membranes were washed and Peroxidase-conjugated second antibody (Santa Cruz, Santa Cruz, Calif.) was added for 1 hour. The membrane was again washed and protein detected by incubation for 1 min with ECL detection reagent (Amersham, Arlington Heights, Ill.) followed by exposure to X-ray film. Antibodies used were as follows: phosphospecific Stat 1 antibody, phosphospecific Stat 3 antibody, phosphospecific p44/42 MAP Kinase antibody, and Stat 1 antibody (New England Bio Labs, Beverly, Mass.).

Nuclear Extracts—A previously employed method to prepare nuclear extracts (S. Ikuyama et al., Mol. Endocrinol. 6:1701-1715 (1992)) was modified to prepare extracts from small numbers of cells. Cells were washed, scraped in 1 ml PBS, pelleted in a microfuge, and resuspended in five volumes of Buffer A (10 mM HEPES-KOH, pH 7.9, 10 mM KCl, 1.5 mM MgCl₂, 0.1 mM EDTA) containing 0.3 M sucrose and 2% Tween 40. To release nuclei, they were frozen and thawed once, then repetitively pipetted, 50 to 100 times, using a micropipet with a yellow tip (200 T1 capacity). Samples were overlayed on 1 ml of 1.5 M sucrose in Buffer A and microfuged for 10 min at 4EC. Pelleted nuclei were washed with 1 ml Buffer A, centrifuged for 30 sec, then resuspended in 50 T1 of Buffer B (20 mM HEPES-KOH, pH 7.9, 420 mM NaCl, 1.5 mM MgCl₂, 0.2 mM EDTA, 25% glycerol). Samples were placed on ice for 20 min with occasional vortexing and centrifuged for 20 min at 4EC. The supernatant fraction containing nuclear protein was aliquoted and stored at −70 C. Buffers A and B contained 0.5 mM dithiothreitol (DTT), 0.5 mM phenylmethylsulfonyl (PMSF), 2 ng/ml Pepstatin A and 2 ng/ml Leupeptin. All procedures were performed on ice or at 4° C.

Electrophoretic Mobility Shift Analysis (EMSA)—Oligonucleotides were labeled with [K-³²P]ATP using T4 polynucleotide kinase, then purified on 8% native polyacrylamide gels (S. Ikuyama et al., Mol. Endocrinol. 6:1701-1715 (1992); H. Shimura, et al., Mol. Endocrinol. 8:1049-69 (1994)). Electrophoretic mobility shift analysis were performed as described (S. Ikuyama et al., Mol. Endocrinol. 6:1701-1715 (1992); H. Shimura, et al., Mol. Endocrinol. 8:1049-69 (1994)) using 3 Tg nuclear extract. In some applications, a 100-fold excess of unlabeled oligonucleotide or 1 T1 antiserum to the specific protein in the complex were added to the mixtures during the preincubation period. Radiolabeled double stranded oligonucleotide probe, 50,000 cpm, was added; and the incubation continued for 20 min at 4° C. Mixtures were analyzed on 5% native polyacrylamide gels and autoradiographed.

Results

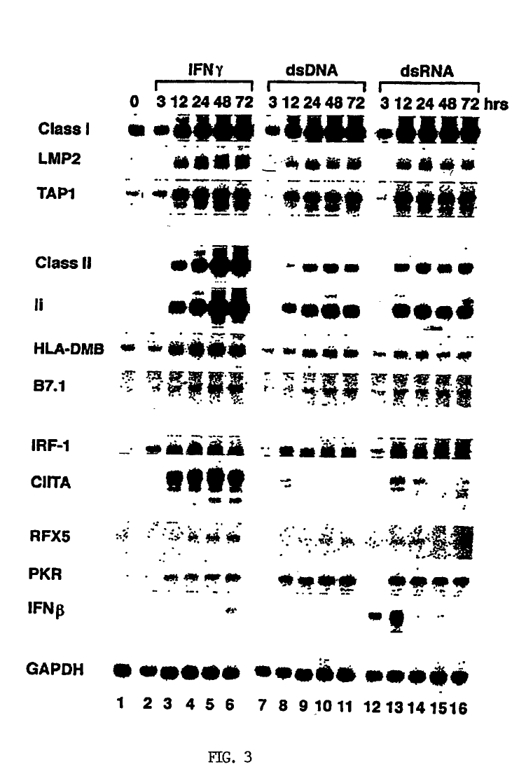

FIG. 3 shows the effects of 100 U/ml KIFN (lanes 2-6) and transfection with 5 Tg dsDNA (lanes 7-11) or dsRNA (lanes 12-16) on genes responsible for antigen presentation. Expression of all these genes is induced by dsDNA or KIFN concomitantly with increased MHC gene expression, suggesting the cells can acquire full capability to present antigen to immune cells. Transfection, KIFN treatment, and Northern analysis 3 to 72 hours after treatment were performed as described in Examples 1 and 2.

Of interest, there is a minimal difference in the ability of either DNA or KIFN to induce changes in RNA levels of these genes as a function of time when evaluated at near maximal stimulation by each agent (i.e., 5 Tg DNA or 100 U/ml KIFN) despite continued evidence of a significant difference in MHC RNA changes, as particularly illustrated by Class II RNA levels (FIG. 3).

KIFN-increased MHC gene expression is mediated by several IFN-inducible genes, including the Class II transactivator (CIITA), RFX5, and the interferon regulatory factor-1 (IRF-1) (B. Mach, et al. Annu. Rev. Immunol. 14: 301-331 (1996); R. M. Ten, et al. C. R. Acad. Sci. III 316: 496-501 (1993)). All three of these genes are induced by KIFN in this system (FIG. 3). The effect of dsDNA on CIITA RNA levels is, however, very different from KIFN, both as a finction of time and level (FIG. 3). The effect of dsDNA and KIFN on RX-5 and IRF-1 RNA levels are less different as a function of time and level; but KIFN is a better inducer of both (FIG. 3).

The dsRNA behaves more like dsDNA than KIFN in having a greater effect on Class I than Class II expression (FIG. 3). Similarly its effects on LMP2, TAP-1, invariant chain (li), HLA-DM [symbol] , and B7 are more like dsDNA than KIFN. Its effect on IRF-1 and CIITA, however, appears to be more a mixture of the effects of dsDNA and KIFN, as a function of both level and time (FIG. 3). This may be explained by the fact that dsRNA, but not dsDNA, increases [symbol] IFN production by the FRTL-5 thyroid cell within 3 hours. Of interest, dsRNA-dependent protein kinase (PKR) (M. J. Clemens & A. Elia, J. Interferon. Cytokine. Res. 17: 503-524 (1997)), which is known to be induced by dsRNA or KIFN, is also induced by dsDNA. In sum, therefore, dsRNA behaves more like dsDNA than KIFN in most respects, with the exception that dsRNA increases [symbol] IFN RNA levels. Since dsRNA is an intermediate in the processing of RNA viruses, this may be an important finctional intermediate in their effects on cells. This is demonstrated in Example 8.

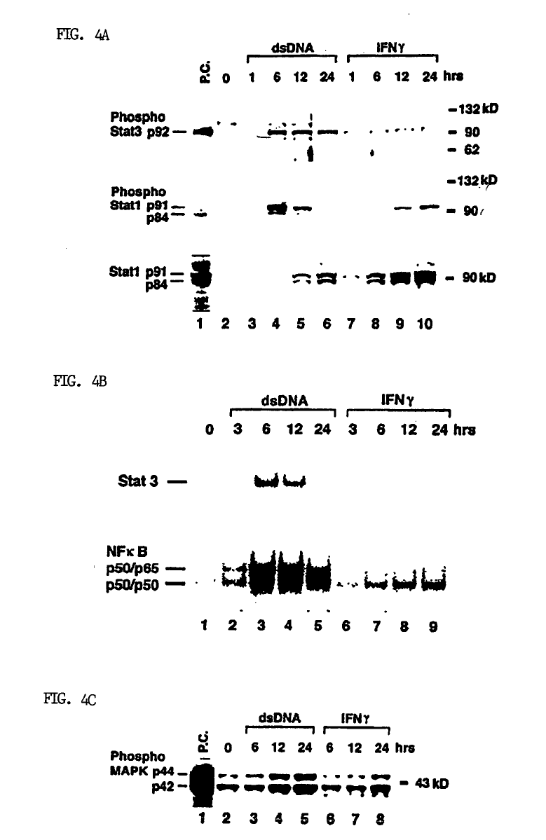

In the next experiment, dsDNA transfection and KIFN treatment of FRTL-5 cells were performed exactly as in Examples 1 and 2. In FIG. 4A, total cell lysate was prepared and Western blot analysis performed as described (A. Hirai, et al. J. Biol. Chem. 272: 13-16 (1997)). Antibodies against phosphorylation-specific Stat 1 (Tyr 701), Stat 3 (Tyr 705) and total Stat 1 are from New England Biolabs (Beverly, Mass.). Lane 1 (P.C.) is a positive control cell lysate from the supplier, New England Biolabs. In FIG. 4B, nuclear protein was prepared and gel shift analysis was performed as described (S. I. Taniguchi, et al., Mol. Endocrinol. 12: 19-33 (1998); P. L. Balducci-Silano, et al., Endocrinology 139: 2300-2313 (1998); V. Montani, et al. Endocrinology 139: 290-302 (1998); K. Suzuki, et al., Endocrinology 139: 3014-3017 (1998); K. Suzuki, at al., Mol. Cell. Biol. in press (1998)). Consensus ODNs for Stat 3 and NF-PB are from Santa Cruz Biotechnology, Santa Cruz, Calif. In FIG. 4C, antibody against phosphorylation-specific p44/p42 MAPK (Erk1and Erk2) (New England Biolabs) was used for Western blotting.

An important mediator of KIFN action is the JAK/STAT signaling pathway (S. Pellegrini & I. Dusanter-Fourth, Eur. J. Biochem. 248: 615-633 (1997)). The dsDNA induced significant phosphorylation of STAT 1 and STAT 3 within 6 hours of transfection and a subsequent increase in total STAT 1 protein which is readily measurable at 12 hours (FIG. 4A). This is very different from the action of KIFN whose effect on STAT 1 and STAT 3 phosphorylation appears significantly lower and more delayed in time in FRTL-5 cells, whereas its effect on total STAT 1 protein is greater and more advanced in time (FIG. 4A). Gel shift analysis using nuclear protein from cells treated with dsDNA uncovered a marked increase in specific binding of STAT 3 to its consensus DNA sequence by comparison to extracts from cells treated with KIFN (FIG. 4B, upper panel).

NF-PB is an important transcription factor for the expression of many genes including the Class I gene; it is composed of two subunits termed p50 and p65 (S. I. Taniguchi, et al., Mol. Endocrinol. 12: 19-33 (1998); R. M. Ten, et al., C. R. Acad. Sci. III 316: 496-501 (1993)). Nuclear translocation and binding of NF-PB subunits requires proteolytic degradation of the IPB/NF-PB cytoplasmic complex by proteosomes and subunit phosphorylation (V. J. Palombella, et al., Cell. 78: 773-785 (1994)). Significantly increased binding, and presumably formation, of a p50/p65 and a p50 homodimer to a consensus NF-PB oligonucleotide binding site was measurable using nuclear extracts from cells transfected with dsDNA within 3 hours. In contrast, KIFN treatment of cells induced a significantly lesser level of p50 homodimer, and particularly p50/p65 heterodimer formation and binding, and a very different effect as a finction of time (FIG. 4B, lower panel).

Another difference between dsDNA and KIFN action was noted on phosphorylation of MAPK (FIG. 4C). Phosphorylation appeared to occur faster as a function of time and appeared to involve a quantitatively larger fraction of the protein pool.

The polynucleotides used in these experiments were poly(dI)/poly(dC) and poly(I)/poly(C) polymers made by Pharmacia Biotech, Piscataway, N.J. The same results were obtained, however, using sonicated salmon sperm DNA (Stratagene, La Jolla, Calif.), bacterial DNA or calf thymus DNA (Sigma, St. Louis, Mo.), FRTL-5 cell genomic DNA, viral DNA from human herpes simplex virus, viral DNA oligonucleotides from HIV, HTLV-1, foamy virus, and cytomegalic virus (CMV), as well as DNA from plasmid vectors pcDNA3 and pRc/RSV, used with or without methylation.

These data were the same independent of the transfection procedure used, i.e. DEAE Dextran or electroporation. Additionally, they were in all respects duplicated in experiments using human hepatoblastoma cell line, HuH7; NIH 3T3 cells; the Pre B cell line, WEHI231; the macrophage line, P381 D1; human muscle cells, SkMC; human endothelial cells, HUVEC; mouse smooth muscle cells, C2C12; and primary cultures of mouse spleen dendritic cells.

Thus, as in Example 1, the phenomenon was not cell specific. Further, the effect of ds nucleic acids was evident in cell types of tissues or organs where autoimmune disease is known to occur or be a part of the tissue damage process, e.g. hepatitis, atherosclerosis, Graves' disease, thyroiditis, psoriasis, systemic lupus and related collagen diseases, alopecia, and myositis, to name but a few. Moreover, the increases in lymphocytes, macrophages, and dendritic cells indicates immune cells can be directly and similarly effected by the ds nucleic acid. Finally the phenomenon is not restricted to normal cells such as the FRTL-5 cell line which is fully functional and under hormonal control, but is also evident in cells which have greater or lesser levels of a transformed phenotype.

To summarize, double-stranded polynucleotide acts significantly differently from KIFN in its effects on key components of the protein processing and transcriptional activation events involved in the expression of MHC and other genes, very likely contributing to differences in their overall finctional effect. The ds polynucleotides increase or activate a multiplicity of genes important for antigen presentation but also important cell growth and finction and involved in onogene transformation.

### Example 3:

THE ACTION OF ANY DOUBLE STRAND VIRAL, BACTERIAL, OR MAMMALIAN NUCLEIC ACID IS NOT ONLY DIFFERENT FROM KIFN WITH RESPECT TO INCREASES IN MHC GENE EXPRESSION AND GENE EXPRESSION, THEY ARE ADDITIVE WITH KIFN AND ARE MIMICKED BY TISSUE DAMAGE INDUCED BY EXOGENOUS INSULTS

The autoimmune process involves an interactive and spiraling cascade of events involving the target tissue and immune cells (G. F. Bottazzo, et al., Lancet 2: 1115-1119 (1983); I. Todd, et al., Annals N. Y Acad. Sci. 475: 241-249 (1986); D. S. Singer, et al., Crit. Rev. Immunol. 17: 463-468 (1997); M. Londei, et al., Nature 312: 639-641 (1984); Shimojo, N. et al., Proc. Natl. Acad. Sci. U.S.A. 93: 11074-11079 (1996); D. S. Singer & J. E. Maguire, CRC Crit. Rev. Immunol. 10: 235-257 (1990); S. I. Taniguchi, et al., Mol. Endocrinol. 12: 19-33 (1998); P. L. Balducci-Silano, et al., Endocrinology 139: 2300-2313 (1998); V. Montani, et al. Endocrinology 139: 290-302 (1998); I. A. York & K. L. Rock, Annu. Rev. Immunol. 14: 369-396 (1996); J. Pieters, Curr. Opin. Immunol. 9: 89-96 (1997); B. Mach, et al., Annu. Rev. Immunol. 14: 301-331 (1996); R. M. Ten, et al., C. R. Acad. Sci. III 316: 496-501 (1993)). If dsDNA and KIFN are separate activators of target tissue MHC genes with different mechanisms, as suggested above, their effects should be additive at maximal concentrations of each. Further, there are multiple ways for cells to be exposed to double-stranded polynucleotides other than by viral infection. One of these is injury-induced escape and migration of self genomic or mitochondrial DNA into the cytoplasm (C. W. Moffett & C. M. Paden, J. Neuroimmunol. 50: 139-151 (1994)). Moreover, increased Class I and Class II expression was reported following tissue damage in vivo even in IFN or IFN receptor knockout mice (P. F. Halloran, et al., Transplant Proc. 29: 1041-1044 (1997)).

The following experiments were, therefore, performed to evaluate the effect of ds polynucleotides and KIFN alone or together on the expression or activation of genes important for antigen presentation as well as MHC expression. Additionally they were performed to examine the possibility that tissue damage, in this case induced by electrical overstimulation during electroporation, could act like ds nucleic acids and whether the tissue damage was associated with ds genomic DNA leaking from the nucleus. We again used rat thyrocytes as a model; but validated the results in a multiplicity of cells as described in Example 1.

Experimental Protocol

Cells—Rat FRTL-5 thyroid cells were a fresh subclone (F1) with all properties described (F. S. Ambesi-Impiombato, U.S. Pat. No. 4,608,341 (1986); L. D. Kohn, et al., U.S. Pat. No. 4,609,622 (1986); L. D. Kohn, et al., Methimazole derivatives and tautomeric cyclic thiones to treat autoimmune disease. U.S. Patent application submitted Aug. 31, 1998; D. S. Singer., et al., U.S. Pat. No. 5,556.754, issued Feb. 17, 1996; P. L. Balducci-Silano, et al., Endocrinology 139: 2300-2313 (1998); V. Montani, et al., Endocrinology 139: 290-302 (1998); M. Saji, et al., J. Biol. Chem. 272: 20096-20107 (1997)). They were grown in 6H medium consisting of Coon's modified F12 medium, 5% heat-treated, mycoplasma-free, calf serum, 1 mM nonessential amino acids, and a six hormone mixture: bovine TSH (1×10⁻¹⁰M), insulin (10 Tg/ml), cortisol (0.4 ng/ml), transferrin (5 Tg/ml), glycyl-L-histidyl-L-lysine acetate (10 ng/ml), and somatostatin (10 ng/ml). Cells, were fed every 2-3 days and passaged every 7-10. In some experiments, cells were treated with 100 U/ml rat KIFN for the last 48 hour of culture before or after transfection with ds polynucleotide.

Transfection—All procedures used 10 cm dishes. Transfection with Lipofectamine Plus (GIBCO BRL, Gaithersburg, Md.) was as in Examples 1 and 2. Thus, 5 Tg DNA was mixed with 30 ml of Plus reagent and 750 T1 of serum-free medium, then incubated for 15 min at room temperature. A mixture of 30 T1 of Plus reagent and 750 T1 of serum-free medium was then prepared and mixed with the above DNA-containing mixture. Cells were washed with serum-free medium and the above mixture was added. Three hours later, medium was replaced with serum-containing, normal culture medium.

For electroporation, cells were suspended with different amounts of DNA in 0.8 ml of DPBS and were pulsed with increasing voltages, various capacitances, and a Gene Pulser (Bio-Rad, Richmond Va.). They were then returned to the culture dish and cultured in growth medium as described.

Nucleic Acids—The following polynucleotide was used in these experiments: poly(dI)/poly(dC). Experiments with poly(I)/poly(C) yielded the same results. The same results were also obtained using sonicated salmon sperm DNA (Stratagene, La Jolla, Calif.), bacterial DNA or calf thymus DNA (Sigma, St. Louis, Mo.), and FRTL-5 cell genomic DNA. Genomic DNA was purified using a Wizard Genomic DNA purification Kit (Promega, Madison, Wis.). Viral DNA from Human Herpes Simplex virus and viral DNA oligonucleotides from HIV, HTLV-1, Foamy virus, and cytomegalic virus (CMV) as well as the plasmid vectors pcDNA3 and pRc/RSV, used with or without methylation, also duplicated the results with the ds synthetic polynucleotides. Plasmid DNAs were purified using EndoFree Plasmid Maxi Kits (QIAGEN, Valencia, Calif.).

Northern Analysis—Total RNA was prepared and Northern analysis performed as described (M. Saji, et al., J. Clin. Endocrinol. Metab. 75: 871-878 (1992); P. L. Balducci-Silano, et al., Endocrinology 139: 2300-2313 (1998); V. Montani, et al., Endocrinology 139: 290-302 (1998); S.-I. Taniguchi, et al., Mol. Endocrinol. 12: 19-33 (1998)). Probes for MHC class I and class II are those described (M. Saji, et al., J. Clin. Endocrinol. Metab. 75: 871-878 (1992); P. L. Balducci-Silano, et al., Endocrinology 139: 2300-2313 (1998); V. Montani, et al., Endocrinology 139: 290-302 (1998); S.-I. Taniguchi, et al., Mol. Endocrinol. 12: 19-33 (1998)). The glyceraldehyde phosphate (GAPDH) probe used was cut from a pTR1-GAPDH-Rat template (Ambion, Tex.). The pTR1-GAPDH rat template was digested using restriction enzymes Sac I and BamHI to release a 316 bp fragment. The fragment was cut from agarose gels, purified using JetSorb Kit (PGC Science, Frederick, Md.), and subcloned into a pBluescript SK(+) vector at the same restriction site.

For Polymerase Chain Reactions (RT-PCR), the MHC class II DNA probe used a sense primer having the nucleotide sequence, 5′-AGCAAGCCAGTCACAGAAGG-3′ (SEQ ID NO:17), and an antisense primer with the sequence, 5-GATTCGACTTGGAAGATGCC-3′ (SEQ ID NO:18), which amplified a 546 bp product, from between 74 and 619 bp of the class II sequence. Both primer regions are highly conserved in the class II nucleotide and protein sequence. Contamination of genomic DNA in total RNA preparations was tested using PCR primers which detect an intronic sequence of rat CIITA genome DNA (M. Pietrarelli et al., manuscript in preparation).

Results

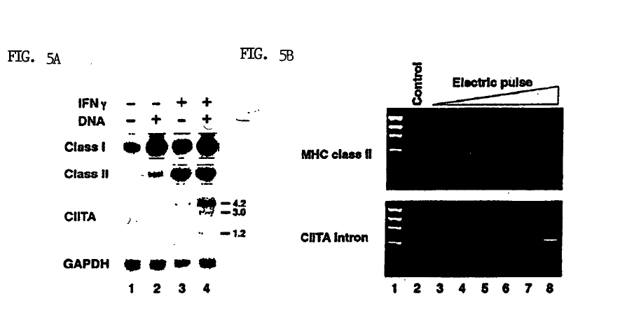

In FIG. 5A, dsDNA transfection and KIFN treatment of FRTL-5 cells were performed exactly as in Examples 1 and 2. Northern analysis was performed 48 hours after treatment. In FIG. 5B, we exposed FRTL-5 cells to a high electic pulse. FRTL-5 cells, 5×10⁶ cells, in Dulbecco's phosphate buffered saline, were pulsed once with a GENE PULSER electroporation apparatus (BioRad, Richmond, Calif.) set at 0.3 kV and at capacitances of 0.25, 25, 125, 250, and 960 TF or twice with a capacitance of 960 TF (lanes 3-8). Cells were washed with medium, returned to 10 cm dish and cultured 48 hours until RNA was recovered. Damage was estimated microscopically, by trypan blue exclusion, and plating efficiency after pulsing; at 960 TF, 60% of cells were fused or dead. RT-PCR of Class II was performed as described in the experimental protocal of this Example and Example 2. Contamination of genomic DNA in total RNA preparations was tested using PCR primers which detect an intronic sequence of rat CIITA genomic DNA (Pietrarelli, et al., manuscript in preparation)

With progressively increased levels of pulsing, increased expression of MHC RNA was noted (FIG. 5B, lanes 6-8). Using total RNA, PCR, and primers to amplify genomic intron sequences without first strand synthesis, we could successfully amplify intron sequence in parallel to the strength of electric pulse and the appearance of MHC RNA (FIG. 5B, lanes 6-8), i.e., leaked self genomic DNA correlated with the increase in MHC expression. The data in FIG. 4 show that ds polynucleotides and KIFN not only are different in their effect on MHC gene expression but also that their effects are additive at maximal stimulatory levels of each. The same results were evident examining the expression or activation of genes important for antigen presentation, growth, or function measured in FIGS. 3 and 4 of Example 2 and using dsRNA or other ds DNA preparations. The data in FIG. 5 show that tissue damage mimics the action of ds nucleic acids.

We conclude that any double-stranded polynucleotide, introduced in the cytoplasm by infection or leakage of self DNA, can directly induce MHC expression, and, concomitantly, increase or activate other essential factors important for antigen presentation. We suggest this can turn normal cells into antigen presenting cells with abnormally expressed MHC genes and thereby enable them to present autoantigens or foreign antigens to our immune cell repertoire. This may be induced by viral dsDNA, viral dsRNA produced by replication of an RNA virus, or perhaps virally- or environmentally-induced tissue damage. We suggest this is a plausible mechanism to explain the action of viruses to induce autoimmunity, that is consistent with the evidence that viruses trigger autoimmune disease by bystander activation of T cells not molecular mimicry (M. S. Horowtiz, et al., Nature. Med. 4: 781-785 (1998); C. Benoist & D. Mathis, Nature 394: 227-228 (1998); H. Wekerle, Nature Med. 4: 770-771 (1998)). The data are consistent with the evidence indicating that, although the virus infection of the target tissue presents self antigens to activate T cells in the normal repertoire, these produce the cytokine (IL18/IL-12/KIFN) cascade which furthers the autoimmune process. An additive or, perhaps, even synergistic increase in MHC gene expression in the target tissue, induced by the initial dsDNA insult and the reactive immune cell production of cytokines and KIFN, may convert a protective process to a process causing autoimmune disease. This process may have additional impacts. It may contribute to the development of autoimmunity when plasmid DNA is introduced during gene therapy (A. K. Yi, et al., J. Immunol. 156: 558-564 (1996); D. M. Klinman, et al., Proc. Natl. Acad. Sci. U.S.A. 93: 2879-2883 (1996)). It may also be important when dsDNA is used in vaccinations. In vaccination, abnormal MHC gene expression at the site of injection might help long-term antigen presentation.

Studies of tumor cells have shown that dsDNA is present in the cytoplasm (A. Solage and R. Laskov, Eur. J. Biochem. 60: 23-33 (1975); R. Hegger and H. Abken, Physiol. Chem. Phys. Med. NMR 27: 321-328 (1995)). Were dsDNA in the cytoplasm to increase 90K synthesis as well as enhance Class I levels (which is reasonable since Class I levels can increase on the surface of tumor cells) this would subject the tumor cell to immune regulation similar to a cell invaded by a bacteria or virus or subjected to tissue injury (H. Wekerle, Nature Medicine 4: 770-771 (1998); C. Benoist & D. Mathis, Nature 394: 227-228 (1998); P. E. Thorsness & E. R. Weber, Int. Rev. Cytol. 165: 207-234 (1996); C. W. Moffett & C. M. Paden, J. Neuroimmunol. 50: 139-151 (1994)). This raises the possibility that ds nucleic acids play an important role in the immune response to the oncogene-induced injury. The ds nucleic acids induce a controlled immune response, similar to a viral infection, causing bystander activation of the immune system and cell destruction by cytotoxic immune cells or antibody mediated destruction (H. Wekerle, Nature Medicine 4: 770-771 (1998); C. Benoist & D. Mathis, Nature 394: 227-228 (1998)). The ds nucleic acids become not only a means of host defense against oncogene transformation but also a means of therapeutic immuno-intervention to enhance tumor killing by bystander activation of dormant autoreactive cytotoxic cells. This possibility is supported by studies of the effect of ds nucleic acids on the 90K tumor-associated immunostimulator to be described in Example 6.

### Example 4

DRUGS WHICH SUPPRESS AUTOIMMUNITY IN VIVO, METHIMAZOLE OR 5-PHENYLMETHIMAZOLE, INHIBIT THE ABILITY OF DOUBLE STRAND POLYNUCLEOTIDES TO INDUCE INCREASES IN MHC GENES, GENES ENCODING ANTIGEN PRESENTING MOLECULES, AND GENES INVOLVED IN THE GROWTH AND FUNCTION OF THE CELL.

The objective of experiments in Examples 4 and 5 was to determine if the ability of double stranded polynucleotides to induce increases in MHC genes and genes encoding antigen presenting molecules (Examples 1 through 3) was related to the development of autoimmunity and the associated control mechanisms affecting the growth and function of cells involved in the autoimmune response. Two approaches were used. First we determined if drugs known to block autoimmunity and transplant rejection in vivo would block the activity of the effect of dsDNA or dsRNA to increase class I/class II gene expression and to increase genes important for antigen presentation to immune cells. This is the subject of Example 4. Second, we directly tested whether the ability of the double stranded polynucleotides to increase MHC class I, cause aberrant expression of MHC class II, and increase antigen presenting molecules in cells would, in a model system, cause disease. This is the subject of Example 5. The results described in both examples affirm the importance of this phenomenon to the development of autoimmunity. Moreover, they indicate that the phenomenon is drug sensitive and can therefore be used to screen for other agents effective as drugs to treat autoimmune disease. Further, the effect of double strand polynucleotides on gene expression can be used to determine or screen for the existence of other genes whose expression is increased during an autoimmune response and for genes whose expression must be controlled in order to regulate the growth and function of the cell, tissue, or organ during the autoimmune response. Identification of these will provide alternative methodologies to develop drugs to control autoimmune responses important as host defense mechanisms and prevent excess responses leading to expression of a disease state. They will additionally identify host genes that may be useful to control the effect on cell growth and function of viral, bacterial, or other infections, of exogenous agents causing tissue damage, or of oncogene transformation, as will also be evident from Examples 6 through 8.

We used methimazole and 5-phenylmethimazole in this experiment. In U.S. Pat. No. 5,556,754, methimazole (MMI) was described to suppress autoimmunity in a model of systemic lupus erythematosus (SLE). MMI was already well known to treat patients with autoimmune hyperthyroidism (Graves' disease) (D. S. Cooper, New Engl. J. Med. 311: 1353-1362 (1984); W. L. Green, in Werner and Ingbar's The Thyroid: A Fundamental Clinical Text, 6^{th} Edition, L. Braverman and R. Utiger (eds), J. B. Lippincott Co., p. 234 (1991)). MMI has also been used to treat psoriasis (U.S. Pat. No. 5,310,742, issued May 10, 1994) and juvenile diabetes (W. Waldhausl, et al., Akt. Endocrin. Stoffw. 8: 119 (1987)). Isothiourea compounds have been described to treat autoimmune diseases in host vs graft disease (British Patent 592,453, Durant et al.).

In recent work searching for MMI derivatives effective to treat autoimmune diseases, a novel set of autoimmune agents, tautomeric cyclic thiones, was described, a potent example of which was 5-phenylmethimazole (compound 10) (L. D. Kohn, et al. Methimazole derivatives and tautomeric cyclic thiones to treat autoimmune disease. U.S. Patent application submitted Aug. 31, 1998). This group of agents had been described for use in studies of thione-thiol equilibria (Kjellin and Sandstrom, Acta Chemica Scandinavica, 23: 2879-2887 and 2888-2899 (1969)). The 5 phenylmethimazole derivative (compound 10) was found to suppress the development of Diabetes in female NOD mice, and systemic lupus erythematosus (SLE) in female (NZBxNZW)F₁ mice (L. D. Kohn, et al. Methimazole derivatives and tautomeric cyclic thiones to treat autoimmune disease. U.S. Patent application submitted Aug. 31, 1998). It was found to be 10- to 100-fold more potent than MMI. Like MMI, however, its action was linked to suppression of K-interferon (KIFN)-induced major histocompatibility complex (MHC) Class I and Class II gene expression and basal MHC gene expression as evidenced by measurements of surface levels of MHC antigens, RNA levels, binding to specific elements of the MHC Class I and Class II 5′-flanking regions, and MHC Class I and Class II promoter expression using both transient and stable transfection procedures (L. D. Kohn, et al. Methimazole derivatives and tautomeric cyclic thiones to treat autoimmune disease. U.S. Patent application submitted Aug. 31, 1998; D. S. Singer, et al., U.S. Pat. No. 5,556,754, issued Feb. 17, 1996; P. L. Balducci-Silano, et al., Endocrinology 139: 2300-2313 (1998); V. Montani, et al., Endocrinology 139: 290-302 (1998); M. Saji, et al., J. Biol. Chem. 272: 20096-20107 (1997)).

Of particular interest, U.S. Pat. No. 3,641,049 (Sandstrom et al., issued Feb. 8, 1972) disclosed that some tautomeric cyclic thiones, particularly 1, 3-dimethylphenylimadazoline-2-thione exhibits antiviral properties against herpes simplex and vaccinia viruses. Thus, since dsDNA and dsRNA increase Class I/Class II gene expression, increase genes important for antigen presentation to immune cells, and mimic infections with viral agents, it is reasonable to anticipate that drugs which suppress the dsDNA or dsRNA effect, may be useful to suppress viral action or, conversely, some antiviral drugs will suppress the effect of dsDNA or dsRNA on the MHC or antigen presenting genes linked to autoimmunity.

Experimental Protocol

Rat FRTL-5 thyroid cells were a fresh subclone (F1) with all properties described (F. S. Ambesi-Impiombato, U.S. Pat. No. 4,608,341 (1986); L. D. Kohn, et al., U.S. Pat. No. 4,609,622 (1986); L. D. Kohn, et al., Methimazole derivatives and tautomeric cyclic thiones to treat autoimmune disease. U.S. Patent application submitted Aug. 31, 1998; D. S. Singer, et al., U.S. Pat. No. 5,556,754, issued Feb. 17, 1996; P. L. Balducci-Silano, et al., Endocrinology 139: 2300-2313 (1998); V. Montani, et al., Endocrinology 139: 290-302 (1998); M. Saji, et al., J. Biol. Chem. 272: 20096-20107 (1997)). They were grown in 6H medium consisting of Coon's modified F12 medium, 5% heat-treated, mycoplasma-free, calf serum, 1 mM non-essential amino acids, and a six hormone mixture: bovine TSH (1×10⁻¹⁰M), insulin (10 Fg/ml), cortisol (0.4 ng/ml), transferrin (5 Fg/ml), glycyl-L-histidyl-L-lysine acetate (10 ng/ml), and somatostatin (10 ng/ml). Cells, were fed every 2-3 days and passaged every 7-10.

FRTL-5 cells were grown in 10 cm dishes to a density of 2×10⁶ cells. One set of cells was immediately used in the assays; the second set was maintained 5 days in medium without TSH (5H) medium before use. Cells were fed fresh medium and treated with 5 mM MMI, 5 mM 2-mercaptoimidazole (Compound 3 in L. D. Kohn, et al., Methimazole derivatives and tautomeric cyclic thiones to treat autoimmune disease. U.S. Patent application submitted Aug. 31, 1998) or 0.5 mM 5-phenylmethimazole (Compound 10 in L. D. Kohn, et al., Methimazole derivatives and tautomeric cyclic thiones to treat autoimmune disease. U.S. Patent application submitted Aug. 31, 1998) Compound 10 is the most active antimmune drug, MMI the standard, and compound 3 an inactive control (L. D. Kohn, et al., Methimazole derivatives and tautomeric cyclic thiones to treat autoimmune disease. U.S. Patent application submitted Aug. 31, 1998). Treatment was for 48 hours. Cells were then transfected with 5 ug dsDNA or dsRNA using Lipofectamine Plus (GIBCO BRL, Gaithersburg, Md.) and the protocol detailed in Examples 1-3. Total RNA was prepared and Northern analysis performed for the noted genes: MHC Class I, MHC Class II, a transporter of antigen peptides (TAP-1), the proteasome protein LMP2, invariant chain (Ii), HLA-DM, the 90 kDa immunomodulator, and glyceraldehyde phosphate dehydrogenase (GAPDH) as described in Examples 1 to 3 an in the following references (M. Saji, et al., J. Clin. Endocrinol. Metab. 75: 871-878 (1992); P. L. Balducci-Silano, et al., Endocrinology 139: 2300-2313 (1998); V. Montani, et al., Endocrinology 139: 290-302 (1998); S.-I. Taniguchi, et al., Mol. Endocrinol. 12: 19-33 (1998)). To acquire antigen-presenting ability, a non-immune cell must coordinately activate or induce multiple genes and proteins, other than MHC genes (I. A. York & K. L. Rock, Ann. Rev. Immunol. 14: 369-396 (1996); J. Pieters, Curr. Opin. Immunol. 9: 89-96 (1997); B. Mach, et al., Ann. Rev. Immunol. 14: 301-331 (1996)). These are required for the multiple steps involved in antigen processing or presentation. For example, in the case of MHC Class I, increases in proteasome proteins (i.e., LMP2) and activity are necessary for antigen processing to peptides (I. A. York & K. L. Rock, Ann. Rev. Immunol. 14: 369-396 (1996)). Also, transporters of antigen peptides (TAP) molecules are required to allow antigenic peptides to bind Class I molecules at the cell surface (I. A. York & K. L. Rock, Ann. Rev. Immunol. 14: 369-396 (1996)). In the case of MHC Class II, invariant chain (Ii) and HLA-DM proteins are required to regulate binding of antigen peptides (J. Pieters, Curr. Opin. Immunol. 9: 89-96 (1997); B. Mach, et al., Ann. Rev. Immunol. 14: 301-331 (1996)). The 90K tumor-associated immunostimulator is a member of the scavenger receptor cysteine-rich (SRCR) domain family and is identical to Mac-2 binding protein (Mac-2bp), the dominant ligand for the macrophage-associated S-type lectin, Mac-2 (also known as galectin-3); it is highly homologous to the murine adherent macrophage (MAMA) protein, a membrane glycoprotein that is induced by macrophage adhesion (A. Ullrich, et al., J. Biol. Chem. 269: 18401-18407 (1994); M. M. Lotz, et al., Proc. Natl. Acad. Sci. U.S.A. 90: 3466-3470 (1993); Y. Chicheportiche & P. Vassalli, J. Biol. Chem. 269: 5512-5517 (1994)). Recombinant 90K has been shown to enhance the in vitro generation of cytotoxic effector cells (NK and LAK) from peripheral blood mononuclear cells (PBMC) and to increase IL-2 production by PBMC (A. Ulltrich, et al., J. Biol. Chem. 269: 18401-18407 (1994)). The 90 kDa protein can enhance expression of major histocompatibility (MHC) Class I molecules in human breast cancer cells (C. Natoli, et al., Biochem. Biophys. Res. Commun. 225: 617-620 (1996)). The 90 kDa protein is induced by I and K-interferon (IFN) and by tumor necrosis factor-I, (TNF-I) (S. Iacobelli, et al., Int. J. Cancer. 42: 182-184 (1998); C. Natoli, et al., Brit. J. Cancer. 67: 564-567 (1993); C. Marth, et al. Int. J. Cancer. 59: 808-813 (1994)).

Probes for MHC Class I and Class II are those described (M. Saji, et al., J. Clin. Endocrinol. Metabol. 75: 871-878 (1992); P. L. Balducci-Silano, et al., Endocrinology 139: 2300-2313 (1998); V. Montani, et al., Endocrinology 139: 290-302 (1998); S.-I. Taniguchi, et al., Mol. Endocrinol. 12: 19-33 (1998)). The glyceraldehyde phosphate dehydrogenase (GAPDH) probe used was cut from a pTR1-GAPDH-Rat template (Ambion, Tex.). The pTR1-GAPDH rat template was digested using restriction enzymes Sac I and BamHI to release a 316 bp fragment. The fragment was cut from agarose gels, purified using JetSorb Kit (PGC Science, Frederick, Md.), and subcloned into a pBluescript SK(+) vector at the same restriction site. The probe for rat CIITA is a cloned rat Type III CIITA cDNA fragment in pcDNA3 (K. Suzuki et al., manuscript in preparation). EcoRI is used to release a 4098 bp fragment as the probe. The probe for rat 90K tumor-associated immunostimulator (A. Ullrich, et al., J. Biol. Chem. 269: 18401-18407 (1994)) is a cloned cDNA fragment described in Example 6. The probe for IRF-1 (GeneBank accession No. X14454) was cut from a plasmid kindly provided by Dr. T. Taniguchi, Osaka, Japan. It was cut from pUCIRF-1 which was kindly provided by Dr. Kenji Sugiyama, Nippon Boehringer Ingelheim Vo., Ltd, Hyogo, Japan. Hind III/BamHI was used to release a 2.1 kb fragment. Other probes were made by RT-PCR based on published cDNA sequences using the following ODNs as primers: a 296 base LMP2 probe, TACCGTGAGGACTTGTTAGCG (SEQ ID NO: 1) and ATGACTCGATGGTCCACACC (SEQ ID NO: 2); a 504 base TAP-1 probe, GGAACAGTCGCTTAGATGCC (SEQ ID NO: 3) and CACTAATGGACTCGCACACG (SEQ ID NO: 4); a 635 base invariant chain (Ii) probe, AATTGCAACCGTGGAGTCC (SEQ ID NO: 5) and AACACACACCAGCAGTAGCC (SEQ ID NO: 6); and a 222 base HLA-DM probe, ATCCTCAACAAGGAAGAAGGC (SEQ ID NO: 7) and GTTCTTCATCCACACCACGG (SEQ ID NO: 8). Lipofectamine plus treatment alone served as a control of the transfection procedure.

Results

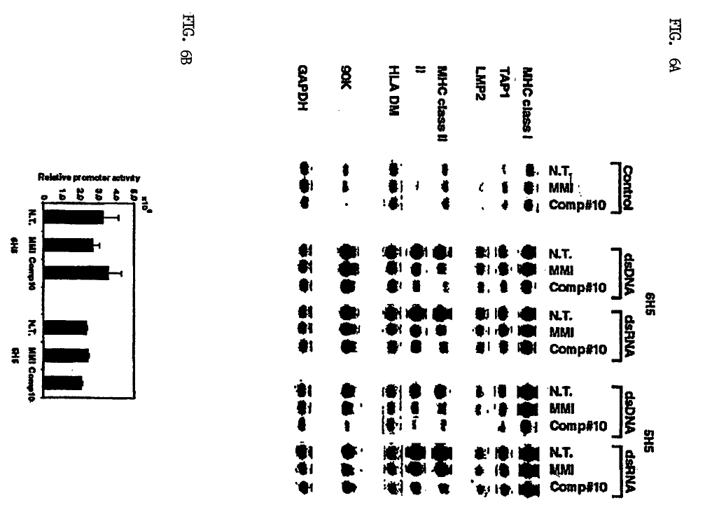

Compound 10, 0.5 mM, significantly decreases the ability of dsDNA to increase MHC Class I, TAP-1, LMP2, MHC Class II, invariant chain, HLA-DM, and 90K tumor-associated immunostimulator gene expression in FRTL-5 thyroid cells exposed to TSH (6H5) or maintained in medium without TSH (5H5) (FIG. 6, Top). The effect of compound 10 seems, however, more pronounced in cells maintained without TSH. The effect of compound 10 is in all cases better than 5 mM MMI (FIG. 6, Top), despite the use of 10-fold lower concentrations. Compound 10 also decreases the ability of dsRNA to increase MHC Class I, TAP-1, LMP2, MHC Class II, invariant chain, HLA-DM, and 90K tumor-associated immunostimulator gene expression in cells exposed to TSH (6H5) or maintained in medium without TSH (5H5) (FIG. 6, Top). Again the effect of compound 10 is better than MMI. There was no effect of 2-mercaptoimidazole, an MMI derivative with no effect on bioactivity as an antiimmune agent (L. D. Kohn, et al., Methimazole derivatives and tautomeric cyclic thiones to treat autoimmune disease. U.S. Patent application submitted Aug. 31, 1998). Treatment with MMI or compound 10 does not affect dsDNA or dsRNA transfection efficiency (FIG. 6, Bottom).

In this experiment (FIG. 6), cells were pretreated with MMI and compound 10 for 2 days before tranfection. A separate experiment involving coincident transfection and treatment with compound 10 also resulted in suppression of the expression of these MHC and antigen-presenting genes. Compound 10 was similarly effective when used to treat endothelia (HUVEC) cells, mouse dendritic cells, and human fibroblasts thranfected with double strand DNA or RNA. The effect was therefore not thyroid cell restricted.

Thus, a drug which suppresses interferon induced MHC Class I and Class II, as well as basal levels of Class I (L. D. Kohn, et al. Methimazole derivatives and tautomeric cyclic thiones to treat autoimmune disease. U.S. Patent application submitted Aug. 31, 1998) suppresses the ability of dsDNA or dsRNA to induce Class I/Class II gene expression and to modulate genes important for antigen presentation to immune cells. Compound 10 is much better than MMI as also described in the separate study (L. D. Kohn, et al. Methimazole derivatives and tautomeric cyclic thiones to treat autoimmune disease. U.S. Patent application submitted Aug. 31, 1998) and 2-mercaptoimidazole has no effect, also in agreement with its potency in suppressing autoimmune disease (L. D. Kohn, et al. Methimazole derivatives and tautomeric cyclic thiones to treat autoimmune disease. U.S. Patent application submitted Aug. 31, 1998).

The development of organ- or tissue-specific autoimmune diseases is associated with abnormal expression of major histocompatibility (MHC) Class I and aberrant expression of MHC Class II antigens on the surface of cells in the target organ or tissue (G. F. Bottazzo, et al., Lancet 2: 1115-1119 (1983); I. Todd, et al., Annals N.Y. Acad. Sci. 475: 241-249 (1986); J. Guardiola & A. Maffei, Crit. Rev. Immunol. 13: 247-268 (1993); D. S. Singer, et al., Crit. Rev. Immunol. 17: 463-468 (1997)). Abnormal expression of MHC molecules on these non-immune cells can cause them to mimic antigen presenting cells and present self-antigens to T cells in the normal immune cell repertoire (M. Londei, et al., Nature 312: 639-641 (1984); N. Shimojo, et al., Proc. Natl Acad. Sci. U.S.A. 93: 11074-11079 (1996)). This leads to a loss in self tolerance and the development of autoimmunity (G. F. Bottazzo, et al., Lancet 2: 1115-1119 (1983); I. Todd, etal. Annals N.Y. Acad. Sci. 475: 241-249 (1986); J. Guardiola & A. Maffei, Crit. Rev. Immunol. 13: 247-268 (1993); D. S. Singer, et al., Crit. Rev. Immunol. 17: 463-468 (1997); M. Londei, et al., Nature 312: 639-641 (1984); N. Shimojo, et al., Proc. Natl. Acad. Sci. U.S.A. 93: 11074-11079 (1996)).

Viral infections can ablate self tolerance, mimic immune responses to self antigens, and to cause autoimmune disease (J. Guardiola, & A. Maffei, Crit. Rev. Immunol. 13: 247-268 (1993); R. Gianani & N. Sarvetnick, Proc. Natl. Acad. Sci. U.S.A. 93: 2257-2259 (1996); M. S. Horowitz, et al. Nature Medicine 4: 781-785 (1998); H. Wekerle, Nature Medicine 4: 770-771, (1998); C. Benoist & D. Mathis, Nature 394: 227-228 (1998)). One mechanism by which a viral infection could ablate self-tolerance is the induction of KIFN production by immune cells (I. Todd, et al. Annals. N.Y. Acad. Sci. 475: 241-249 (1986); J. Guardiola & A. Maffei, Crit. Rev. Immunol. 17: 463-468 (1997); M. S. Horowitz, et al. Nature Medicine 4: 781-785 (1998); H. Wekerle, Nature Medicine 4: 770-771, (1998) C. Benoist & D. Mathis, Nature 394: 227-228 (1998)). Although KIFN can certainly increase MHC gene expression in the target tissue (J. P.-Y. Tlng & A. S. Baldwin, Curr. Opin. Immunol. 5: 8-16 (1993)), this does not address the mechanism by which a tissue or target cell viral infection recruits and activates immune cells to produce KIFN. Additionally, it is unlikely that KIFN alone causes autoimmunity, since its administration does not induce typical autoimmune disease (F. Schuppert, et al., Thyroid 7: 837-842 (1997)). Moreover, generalized KIFN production by immune cells cannot account for cell-specific autoimmunity, i.e. destruction of pancreatic [symbol] but not I cells in insulin-dependent diabetes mellitus or involvement of only thyroid follicular cells, not parafollicular C cells, in autoimmune Graves' disease (G. F. Bottazzo, et al., Lancet 2: 1115-1119 (1983); I. Todd, et al., Annals. N.Y. Acad. Sci. 475: 241-249 (1986); N. Shimojo, et al., Proc. Natl. Acad. Sci. U.S.A. 93: 11074-11079 (1996); A. K. Foulis, et al., Diabetologia 30: 333-343 (1987)). In the present experiments, cells were not treated with KIFN; therefore, KIFN cannot be construed as mechanistically involved. The effect of compound 10 or MMI on dsDNA- or dsRNA-induced changes is not caused by interferon or other immune cell produced or induced cytokines. Rather it is more likely to be related to the effects on basal Class I activity which are perturbed by the dsDNA or dsRNA introduced via the viral infection.

Recent work (M. S. Horowitz, et al., Nature Medicine 4: 781-785 (1998); H. Wekerle, Nature Medicine 4: 770-771 (1998); C. Benoist & D. Mathis, Nature 394: 227-28 (1998) has suggested that viral triggering of diverse autoimmune diseases including rheumatoid arthritis, insulin dependent diabetes, and multiple sclerosis is caused by local viral infection of the tissue not molecular mimicry. It is suggested this involves MHC genes and results in presentation of self-antigens, the exact effect of the dsDNA and dsRNA transfections described herein and which were shown to duplicate the action of viral DNA (Examples 1 and 2). Thus, since dsDNA and dsRNA increase Class I/Class II gene expression, increase genes important for antigen presentation to immune cells, and mimic infections with viral agents, it is reasonable to anticipate that drugs which suppress the dsDNA or dsRNA effect, may be useful to suppress viral action or, conversely, some antiviral drugs will suppress the effect of dsDNA or dsRNA on the MHC or antigen presenting genes linked to autoimmunity.

Of particular interest in this respect is that compound 10 is a tautomeric cyclic thione and that U.S. Pat. No. 3,641,049 (Sandstrom et al., issued Feb. 8, 1972) teaches that some tautomeric cyclic thiones, particularly 1, 3-dimethylphenylimadazoline-2-thione, exhibit antiviral properties against herpes simplex and vaccinia viruses. As noted in FIG. 1A, Example 1, we treated rat FRTL-5 thyroid cells with herpes simplex virus or transfected them with various viral DNA preparations, including oligodeoxynucleotides (ODNs) from different viral DNA sequences (FIG. 1). In FIG. 1A, Example 1, herpes simplex infection increased MHC RNA levels in the FRTL-5 cells within 48 hours of infection.

In sum, since drugs which suppress autoimmunity (L. D. Kohn, et al. Methimazole derivatives and tautomeric cyclic thiones to treat autoimmune disease. U.S. Patent application submitted Aug. 31, 1998) can prevent the dsDNA or dsRNA action, it is reasonable to use the assay to screen for agents which are autoimmune agents and do not involve the IFN/cytokine arm of the autoimmune response.

The disclosures of all patents, patent applications, and other publications are incorporated by reference herein as illustrative of the knowledge and skill available to an artisan practicing this invention. In addition, such artisans recognize that obvious changes and modifications to the description provided herein would still constitute practice of this invention within the scope of the appended claims.

### Example 5

AN AUTOIMMUNE DISEASE MIMICKING GRAVES' DISEASE IN HUMANS CAN BE INDUCED IN MICE IMMUNIZED WITH FIBROBLASTS TRANSFECTED WITH DOUBLE STRAND POLYNUCLEOTIDE AND THE THYROTROPIN RECEPTOR

The objective of these experiments was to determine if dsDNA, by increasing Class I/Class II gene expression and by increasing expression or activation of genes important for antigen presentation to immune cells, could induce an autoimmune disease in vivo.

Graves' disease is an autoimmune thyroid disease characterized by the presence of antibodies against the thyrotropin receptor (TSHR) which stimulate the thyroid to cause hyperthyroidism and/or goiter (D. D. Adams, et al., Br. Med. J. 2: 199-201 (1974)). Numerous attempts (G. S. Seetharamaiah, et al., Autoimmunity 14: 315-320 (1993); S. Costagliola, et al., J. Mol. Endocrinol. 13: 11-21 (1994); S. Costagliola, et al., Biochem. Biophys. Res. Commun. 199: 1027-1034 (1994); S. Costagliola, et al., Endocrinology 135: 2150-2159 (1994); A. Marion, et al., Cell. Immunol. 158: 329-341 (1994); N.M. Wagle, et al., Endocrinology 136: 3461-3469 (1995); H. Vlase, et al., Endocrinology 136: 4415-4423 (1995)) to develop a Graves' disease (GD) model by immunizing animals with the extracellular domain of the thyrotropin receptor (TSHR) have largely failed. In most cases antibodies to the TSHR which could inhibit TSH binding were produced and in some cases thyroiditis with a large lymphocytic infiltration developed (G. S. Seetharamaiah, et al., Autoimmunity 14: 315-320 (1993); S. Costagliola, et al., J. Mol. Endocrinol. 13: 11-21 (1994); S. Costagliola, et al., Biochem. Biophys. Res. Commun. 199: 1027-1034 (1994); S. Costagliola, et al., Endocrinology 135: 2150-2159 (1994); A. Marion, et al., Cell. Immunol. 158: 329-341 (1994); N. M. Wagle, et al., Autoimmunity 18: 103-108 (1994); G. Carayanniotis, et al., Clin. Exp. Immunol. 99: 294-302 (1995); G. S. Seethararnaiah, et al., Endocrinology 136: 2817-2824 (1995); N. M. Wagle, et al., Endocrinology 136: 3461-3469 (1995); H. Vlase, et al., Endocrinology 136: 4415-4423 (1995)). However, in no case did the immunization produce thyroid stimulating TSHRAbs which increase thyroid hormone levels, the hall-mark of Graves', nor were the morphologic or histologic features of the disease induced: glandular enlargement, thyrocyte hypercellularity, and thyrocyte intrusion into the follicular lumen. Further, in most studies (G. S. Seetharamaiah, et al., Autoimmunity 14: 315-320 (1993); S. Costagliola, et al., J. Mol. Endocrinol. 13: 11-21 (1994); S. Costagliola, et al., Biochem. Biophys. Res. Commun. 199: 1027-1034 (1994); S. Costagliola, et al., Endocrinology 135: 2150-2159 (1994); A. Marion, et al., Cell. Immunol. 158: 329-341 (1994); N. M. Wagle, et al., Autoimmunity 18: 103-108 (1994); G. Carayanniotis, et al., Clin. Exp. Immunol. 99: 294-302 (1995); G. S. Seetharamaiah, et al., Endocrinology 136: 2817-2824 (1995);N. M. Wagle, et al., Endocrinology 136: 3461-3469 (1995); H. Vlase, et al., Endocrinology 136: 4415-4423 (1995)) the antibodies that inhibited TSH binding were not shown to inhibit TSH activity mediated specifically by the TSH receptor, a feature characteristic of TSH binding inhibitory immunoglobulins (TBIIs) in GD (P. A. Ealey, et al., J. Clin. Endocrinol. Metab. 58: 909-914 (1984); A. Pinchera, et al., in Autoimmunity and the Thyroid, P. G. Walfish, et al., (Eds), Academic Press, New York, pp. 139-145 (1985); G. F. Fenzi, et al., in Thyroid Autoimmunity, A. Pinchera, et al., (Eds.), Plenum Press, New York, pp. 83-90 (1987)).

These studies depended on the ability of the animal to process the TSHR as an extracellular antigen, rather than as a receptor in a finctional state on a cell. They did not take into account the possibility that the TSHR might be presented to the immune system as a result of abnormal major histocompatibility complex (MHC) Class I or Class II expression on thyrocytes, thereby allowing normal immune tolerance to be broken. Thus, several studies have implicated Class I as an important component in the development of autoimmune thyroid disease and in the action of methimazole, a drug used to treat GD (M. Saji, et al., J. Clin. Endocrinol. Metab. 75: 871-878 (1992); L. D. Kohn, et al., Intern. Rev. Immunol. 9: 135-165 (1992); E. Mozes, et al., Science 261: 91-93 (1993); D. S. Singer, et al., J. Immunol. 153: 873-880 (1994); L. D. Kohn, et al., in Thyroid Immunity, D. Rayner and B. Champion (Eds), R. G. Landes Biomedical Publishers, Texas, pp. 115-170 (1995)). In addition, aberrant Class II expression, as well as abnormal expression of Class I molecules, is evident on thyrocytes in autoimmune thyroid diseases (G. F. Bottazzo, et al., Lancet 2: 1115-119) (1983); G. F. Bottazzo, et al., N. Engl. J. Med. 313: 353-360 (1985); I. Todd, et al., Annals N.Y. Acad. Sci. 475: 241-249 (1986)), although the cause and role of aberrant Class II in disease expression was controversial (A. P. Weetman & A. M. McGregor, Endocrinol. Rev. 15: 788-830 (1994)). The sum of these observations raised the possibility that immunization with full length TSHR, in a functional conformation but in the context of abnormal MHC Class I or Class II expression, might lead to the development of GD.

To test the possibility that abnormal MHC expression, as well as a functional, full length TSHR, might result in a Graves'-like disease, N. Shimojo and colleagues transfected full length human TSHR (hTSHR) into murine fibroblasts with or without aberrantly expressed Class II antigen (N. Shimojo, et al., Proc. Natl. Acad. Sci. U.S.A. 93: 11074-11079 (1996); K.-I. Yamaguchi, et al., J. Clin. Endocrinol. Metab. 82: 4266-4269 (1997); S. Kikuoka, et al., Endocrinology 139: 1891-1898 (1998)). Those authors showed that mice immunized with fibroblasts expressing a Class II molecule and holoTSHR, but not either alone, could develop the major features characteristics of Graves' disease (GD): thyroid-stimulating antibodies directed against the TSHR, increased thyroid hormone levels, an enlarged thyroid, and thyrocyte hypercellularity with intrusion into the follicular lumen. The mice additionally develop TBIIs which inhibit TSH-increased cAMP levels in CHO cells stably transfected with the TSHR and appear to be different from the stimulating TSHRAbs, another feature of the humoral immunity in GD. Thus, by immunizing mice with fibroblasts transfected with the human TSHR and a major histocompatibility complex (MHC) Class II molecule, but not by either alone, they had induced immune hyperthyroidism that has the major humoral and histological features of Graves' disease (N. Shimojo, et al., Proc. Natl. Acad. Sci. U.S.A. 93: 11074-11079 (1996); K.-I. Yamaguchi, et al., J. Clin. Endocrinol. Metab. 82: 4266-4269 (1997); S. Kikuoka, et al., Endocrinology 139: 1891-1898 (1998)). The results indicated that the acquisition of antigen-presenting ability on a target cell containing the TSHR can activate T and B cells normally present in an animal and induce an experimental disease with the major features of autoimmune Graves'.

There is evidence linking autoimmune thyroid disease to viral and bacterial infections (Y. Tomer & T. Davies, Endocr. Rev. 14: 107-121 (1993)). The mechanism by which this might occur is unknown (Y. Tomer and T. Davies, Endocr. Rev. 14: 107-121 (1993)). The observation that dsDNA or dsRNA increased Class I/Class II gene expression and increased expression or activation of genes important for antigen presentation to immune cells, together with the evidence noted above that MHC Class I/Class I abnormal expression in the target tissue was involved in the development of an autoimmune disease in vivo, despite a normal immune system, led us to test the hypothesis that ds polynucleotides could induce a Graves' model when they were transfected into fibroblasts expressing the TSHR. We transfected fibroblasts with dsDNA, with the TSHR, or with both. We also transfected cells with dsDNA that had genetically engineered aberrant Class II expression with or without the TSHR.

We questioned whether fibroblasts transfected with dsDNA plus the TSHR, but not either alone, would develop Graves' disease. We questioned whether the presence of the dsDNA plus aberrant Class II and the TSHR would be additive and increase the frequency of Graves' with hyperthyroidism, i.e. from 20 to 25% (N. Shimojo, et al., Proc. Natl. Acad. Sci. U.S.A. 93: 11074-11079 (1996); K.-I. Yamaguchi, et al., J. Clin. Endocrinol. Metab. 82: 4266-4269 (1997); S. Kikuoka, et al., Endocrinology 139: 1891-1898 (1998); to much higher values, because of the additional increase in MHC Class I and antigen presenting molecule expression or activation.

Experimental Protocol

Fibroblasts and Transfection of the TSHR Gene—A murine L. cell fibroblast line, which expresses a hybrid gene containing A^{k} and A^{d} of murine MHC Class II (RT4.15HP&) (R. N. Germain, et al., Proc. Natl. Acad. Sci. U.S.A. 82: 2940-2944 (1985)) was kindly provided by Dr. R. N. Germain (NIAID, NIH) as was the DAP. 3 control cell line, which are Class I-untransfected fibroblasts. The A^{d} determinant is membrane proximal and was shown not to be associated with antigen presentation (R. N. Germain, et al., Proc. Natl. Acad. Sci. U.S.A. 82: 2940-2944 (1985)), i.e. this shuffled I-A^{k} molecule is not different from I-A^{k} in antigen presenting activity. The cloning and characterization of the hTSHR was reported previously (K. Tahara, et al., Biochem. Biophys. Res. Commun. 179: 70-77 (1992). After subcloning into a pSG5 vector (Stratagene, La Jolla, Calif.), the hTSHR was transfected into RT4.15HP or DAP.3 cells together with pMAMneo (Clontech, Palo Alto, Calif.) using LIPOFECTIN (GIBCO BRL, Gaithersburg, Md.), as described by the company. Cells were selected for neomycin resistance using 500 Tg/ml G418 (GIBCO BRL); stable transfectants were selected by their ability to increase cAMP levels in the presence of TSH (W. B. Kim, et al., J. Clin. Endocrinol. Metab. 81: 1758-1767 (1996)). Positive cells were cloned by limiting dilution. Control RT4.15HP cells or DAP.3 cells transfected with pSG5 vector alone were similarly established.

Immunization ofMice with Transfectants and Assay of TSR Antibodies—Seven-week-old female AKR/N (H-2^{k}) mice were intraperitoneally immunized 6 times every 2 weeks with 10⁷ fibroblasts which had been transfected with dsDNA, 5 Tg, 48 hours before immunization and which were pretreated with mitomycin C (N. Shimojo, et al., Proc. Natl. Acad. Sci. U.S.A. 93: 11074-11079 (1996); K.-I Yamaguchi, et al., J. Clin. Endocrinol. Metab. 82: 4266-4269 (1997); S. Kikuoka, et al., Endocrinology 139: 1891-1898 (1998)). The transfection procedure used lipofectamine; control immunizations included cells treated with lipofectamine alone. These mice were chosen because they have the same Class I molecules and a homologous Class II 1-A molecule to that of the fibroblasts containing the transfected Class II and TSHR cDNAs. The time period and protocol duplicated previous studies in which autoimmune hyperthyroidism developed in a significant number of animals. (N. Shimojo, et al., Proc. Natl. Acad. Sci. U.S.A. 93: 11074-11079 (1996)). Two weeks after final immunization, mice were sacrificed and bled. Mouse thyroids were histologically examined by hematoxylin and eosin staining.

Commercial radioimmunoassay (RIA) kits were used to measure the ability of antibodies in the serum to inhibit [¹²⁵I]TSH biding (TBII activity) and to measure serum T3 or T4 levels as previously described (N. Shimojo, et al., Proc. Natl. Acad. Sci. U.S.A. 93: 11074-11079 (1996); K.-I. Yamaguchi, et al., J. Clin. Endocrinol. Metab. 82: 4266-4269 (1997); S. Kikuoka, et al., Endocrinology 139: 1891-1898 (1998)). Stimulating TSHRAb activity was measured using hTSHR-stably-transfected CHO cells (K. Tahara, et al., Biochem. Biophys. Res. Commun. 179: 70-77 (1992); W. B. Kim, et al., J. Clin. Endocrinol. Metab. 81: 1758-1767 (1996)). In brief, 4,000 hTSHR-transfected CHO cells were plated in 96 well flat-bottom plates and cultured for 48 hrs in growth medium. Cells were washed with Hanks Balanced Salt Solution (HBSS) and incubated with 25 T1 protein A-purified IgG (1 mg/ml) and 175 T1 low sodium isotonic HBSS (8 mM Na₂PO₄, 1.5 mM KH₂PO₄, 0.9 mM CaCI₂, and 220 mM sucrose) containing 0.5 mM 3-isobutyl-1-methylxanthine and 1% bovine serum albumin. After a 3 hr incubation at 37 C, supernatants were collected and cAMP was measured with a commercial RIA kit (Yamasa Co. Ltd., Chiba, Japan). IgG was obtained from the sera of all animals within each experimental group.

Flow Cytometry Analysis of Transfectants—As previously described (N. Shimojo, et al., Proc. Natl. Acad. Sci. U.S.A. 93: 11074-11079 (1996); K.-I. Yamaguchi, et al., J. Clin. Endocrinol. Metab. 82: 4266-4269 (1997); S. Kikuoka et al. Endocrinology 139: 1891-1898 (1998)), fibroblasts (10⁶ cells) were incubated with 1 Tg monoclonal anti-I-A^{k} (MHC Class II-specific) or anti-D^{k} (MHC Class I-specific) antibodies obtained from the American Tlssue Culture Collection (ATCC), 10-2.16 or 15-5-S, respectively, or an isotype-specific control monoclonal antibody (Becton Dickinson, Mountainview, Calif.). After 30 min on ice, cells were washed with phosphate buffered saline at pH 7.4 and incubated for 30 min with fluorescein-isothiocyanate (FITC)-conjugated goat anti-mouse IgG (KPL, Gaithersburg, Md.), then analyzed by flow cytometry on a FACScan Cytometer using Cell Quest software (Becton Dickinson).

Northern analysis—Total RNA was prepared and Northern analysis performed for the noted genes: MHC Class I, MHC Class II, a transporter of antigen peptides (TAP1), the proteasome protein LMP2, invariant chain (Ii), HLA-DM, the 90K tumor-associated immunostimulator, and glyceraldehyde phosphate dehydrogenase (GAPDH). The methodology used duplicated that described in Examples 1 to 4 and it described in the following reports (M. Saji, et al., J. Clin. Endocrinol. Metab. 75: 871-878 (1992); P. L. Balducci-Silano, et al., Endocrinology 139: 2300-2313 (1998); V. Montani, et al., Endocrinology 139: 290-302 (1998); S.-I. Taniguchi, et al., Mol. Endocrinol. 12: 19-33 (1998)).

Probes for MHC Class I and Class II are those described in examples 1 through 4 and in the following references (M. Saji, et al., J. Clin. Endocrinol. Metab. 75: 871-878 (1992); P. L. Balducci-Silano, et al., Endocrinology 139: 2300-2313 (1998); V. Montani, et al., Endocrinology 139: 290-302 (1998); S.-I. Taniguchi, et al., Mol. Endocrinol. 12: 19-33 (1998)). The glyceraldehyde phosphate dehydrogenase (GAPDH) probe used was cut from a pTR1-GAPHDH-Rat template (Ambion, Tex.). The probe for rat 90K tumor-associated immunostimulator (A. Ullrich, et al., J. Biol. Chem. 269: 18401-18407 (1994)) is a cloned cDNA fragment as described in Example 6. Other probes were made by RT-PCR based on published cDNA sequences using following ODNs as primers: a 296 base LMP2 probe, TACCGTGAGGACTTGTTAGCG (SEQ ID No: 1 and ATGACTCGATGGTCCACACC (SEQ ID No: 2); a 504 base TAP1 probe, GGAACAGTCGCTTAGATGCC (SEQ ID No: 3) and CACTAATGGACTCGCACACG (SEQ ID No: 4); a 635 base Invariant chain (Ii) probe, AATTGCAACCGTGGAGTCC (SEQ ID No: 5) and AACACACACCAGCAGTAGCC (SEQ ID No: 6) a 22 base HLA-DM probe 1, ATCCTCAACAAGGAAGAAGGC (SEQ ID No: 7) and GTTCTTCATCCACACCACGG (SEQ ID No: 8). Lipofectamine treatment alone served as a control of the transfection procedure.

Results

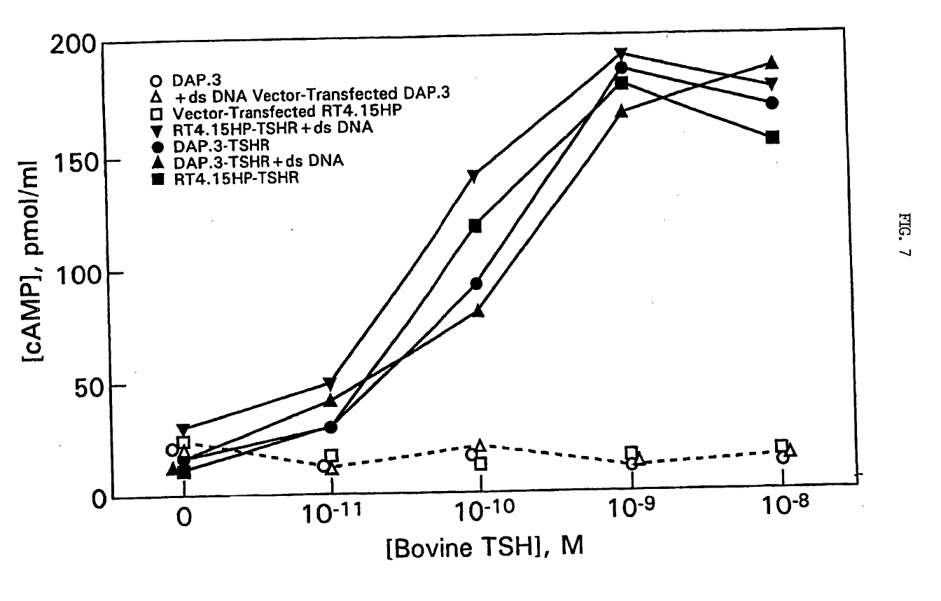

When a murine MHC Class I-transfected fibroblast cell line, RT 4:15HP, or its Class II-untransfected control counterpart, DAP.3, were transfected with human TSHR, both expressed the receptor in a finctional array, exhibiting similar TSH-increased stimulation of the cAMP signal system (FIG. 7). In this experiment, hTSHR-transfected RT4.15HP cells or hTSHR-transfected DAP.3 cells, subjected or not to dsDNA transfection, were stimulated with the indicated concentrations of bovine TSH for 1 hour and the supernatants were collected. cAMP in the supernatant was measured by a commercial RIA kit. The activities of control cells without transfected hTSHR are also presented. Transfection with dsDNA did not alter the TSHR expression (FIG. 7). Control cells without transfected TSHR did not exhibit TSH-responsive adenylylate cyclase activity before or after being transfected with dsDNA. (FIG. 7).

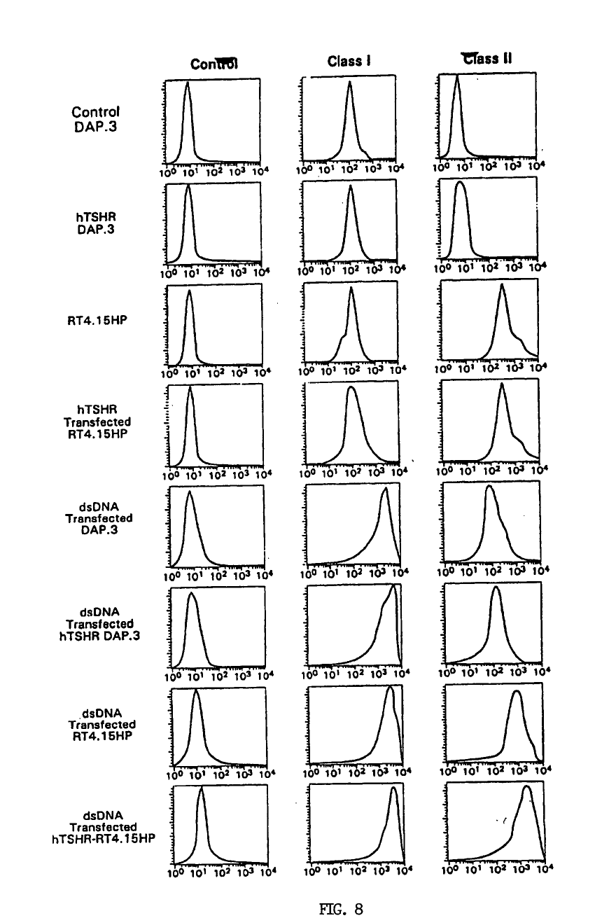

Flow cytometry analysis showed that DAP.3, hTSHR-transfected DAP.3, control vector-transfected RT 4.15HP cells and hTSHR-transfected RT 4.15HP cells expressed comparable levels of Class I molecules on their cell surface as measured by flow cytometry (FACS) analysis (FIG. 8). This experiment was performed as described in the experimental protocol. RT4.15HP or hTSHR-transfected RT4.15HP cells express Class II by comparison to the control DAP.3 or hTSHR-transfected DAP.3 cells, which exhibited no surface expression of Class II antigen (FIG. 8). Flow cytometry analysis showed that dsDNA transfection increased Class I surface expression in each case (FIG. 8). The dsDNA increased Class II expression in the DAP.3 and hTSHR-DAP.3 cells; but the level appeared to be less than in the dsDNA-transfected RT4.15HP or hTSHR-RT4.15HP cells as evidenced by fluorescence intensity changes. The cells were used to immunize AKR/N mice.

As previously reported (N. Shimojo, et al., Proc. Natl. Acad. Sci. U.S.A. 93: 11074-11079 (1996); K.-I. Yamaguchi, et al., J. Clin. Edocrinol. Metab. 82: 4266-4269 (1997); S. Kikuoka, et al., Endocrinology 139: 1891-1898 (1998)), measurements of TBII activity showed that most mice immunized with hTSHR-transfected RT4.15HP cells, for example 90% of mice in Table 1, developed serum TBII activity. This was not true of the mice in the same experiment which were immunized with vector-transfected RT4.15HP cells, DAP.3 cells, or DAP.3 cells expressing hTSHR (Table 1). Twenty-five percent of mice immunized with hTSHR-transfected RT4.15HP cells in the experiment noted in Table 1 also developed hyperthyroidism as evidenced by significantly (P<0.01) elevated serum thyroxine (T4) and triiodothyronine (T3) levels. This was again not true of mice immunized with vector-transfected RT4.15HP cells, DAP.3 cells or DAP.3 cells expressing hTSHR alone (Table 1).

As noted in FIG. 8, dsDNA, when transfected into DAP.3 cells or hTSHR DAP.3 cells, increases Class I as well as Class II expression. One hundred percent of the hTSHR DAP.3 immunized mice transfected with dsDNA, but none of those immunized with DAP.3 without the TSHR, developed serum TBII activity (Table 1). Thirty percent of mice immunized with the hTSHR DAP.3 immunized mice transfected with dsDNA, but none of those immunized with DAP.3 without the TSHR, developed hyperthyroidism as evidenced by significantly (P<0.01) elevated serum thyroxine (T4) and triiodothyronine (T3) levels (Table 1). Immunizing mice with the dsDNA-transfected hTSHR DAP.3 cells results, therefore, in the same Graves' like picture as previously described using cells expressing TSHR plus aberrant Class II (N. Shimojo, et al., Proc. Natl. Acad. Sci. USA 93: 11074-11079 (1996); K.-I. Yamaguchi, et al., J. Clin, Endocrinol. Metab. 82: 4266-4269 (1997); S. Kikuoka, et al., Endocrinology 139: 1891-1898 (1998)). The dsDNA, by increasing Class I and Class II expression, duplicates the effect of aberrant Class II created by genetically overexpressing the Class II gene.

TABLE 1
The effect of dsDNA transfection on the induction of anti-TSHR
TBII antibodies and thyroid function in mice immunized with
TSHR-transfected DAP.3 or TSHR-transfected RT 4.15HP cells by
comparison to control mice immunized with DAP.3 or RT 4.15HP
cells with no transfected TSHR (Control).

| | | Positive | Elevated | | |
|---|---|---|---|---|---|
| | dsDNA | TBII Values | T4 Values | Mean T4 | Mean T3 |
| | Trans- | (% in | (% in | Value ″2 | Value ″2 |
| Cells | fection | Group) | Group) | SD (Tg/dl) | SD (ng/dl) |
| DAP.3 | NO | 0 | 0 | 2.7″0.5 | 57″10 |
| hTSHR | NO | 0 | 0 | 2.3″0.4 | 50″12 |
| DAP.3 | | | | | |
| RT4.15HP | NO | 0 | 0 | 2.2″0.6 | 59″15 |
| hTSHR | NO | 92* | 25* | 12.3″0.8 | 263″30 |
| RT4.15HP | | | | | |
| DAP.3 | YES | 0 | 0 | 3.9″0.7 | 59″15 |
| hTSHR | YES | 100* | 30* | 14.7″1.9 | 230″30* |
| DAP.3 | | | | | |
| RT4.15HP | YES | 0 | 0 | 2.9″0.7 | 50″12 |
| hTSHR | YES | 100* | 75*+ | 19.3″0.9 | 296″30 |
| RT4.15HP | | | | | |

Experiments involved 12 mice in each group. Bold and Starred Values represent a significant increase (P < 0.05 or better) in the experimental animals, by comparison to the control group: DAP.3 with or without dsDNA transfection. The value noted with a (+) represents a significant increase over cells not transfected with dsDNA.

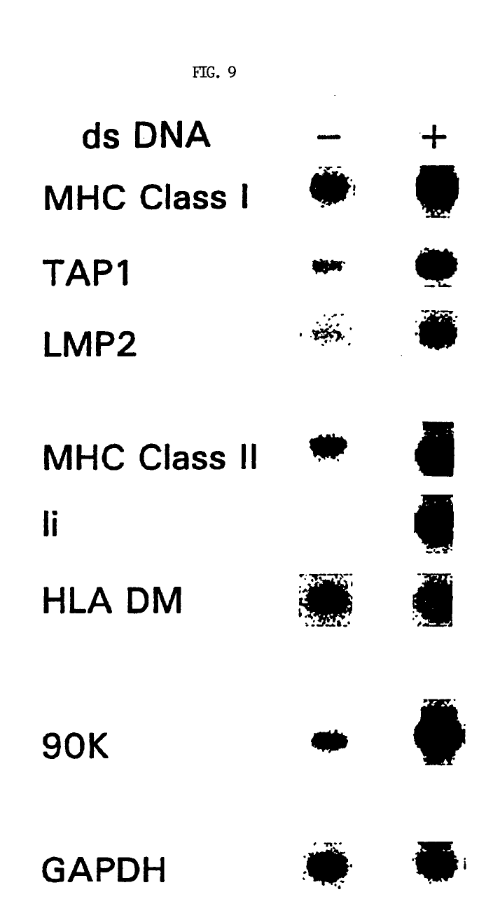

Additionally, 100% of mice immunized with dsDNA-transfected hTSHR RT4.15SHP cells developed serum TBII activity, whereas this was not true of mice immunized with dsDNA-transfected RT4.15HP cells (Table 1). More importantly, immunizing mice with dsDNA-transfected hTSHR RT4.15HP cells resulted in hyperthyroidism in 75% of the mice (Table 1), far more than the 25 to 30% of mice when mice are immunized with DNA-transfected hTSHR DAP.3 cells or hTSHR RT4.15HP cells expressing genetically engineered aberrant Class II alone. These data suggest the dsDNA induction of increased Class I, increased expression of genes important for antigen presentation, or both can significantly increase the appearance of a Graves' like syndrome. FIG. 9 shows that DNA transfection of hTSHR DAP.3 cells results in increased expression of TAP1, LMP2, Invariant chain, HLA DM and 90 kDa immunomodulator as well as MHC Class I and Class II RNA levels. Northern analysis was performed as described in the experimental protocol and in Examples 1 through 4.

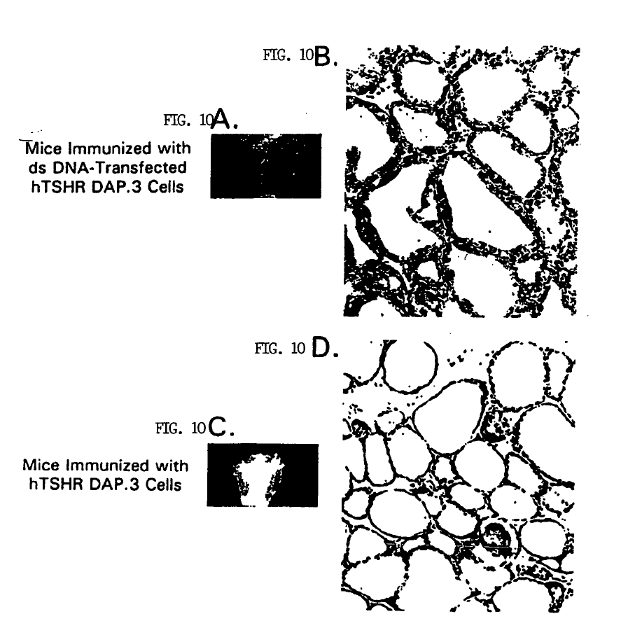

The thyroid glands of mice immunized with dsDNA-transfected hTSHR DAP.3 cells and who developed high serum T4 and T3 showed marked hypertrophy (FIG. 10A) and exhibited thyrocyte hypercellularity with intrusion into the folluclar lumen (FIG. 10B). There was minimal immune cell infiltration, typical of GD rather than thyroiditis (J. E. Ortel, et al., in Werner's The Thyroid, S. H. Ingbar & L. E. Braverman (Eds.), J. B. Lippincott Co., Philadelphia, pp. 651-686 (1986)). All mice immunized with hTSR DAP.3 cells that were not transfected with dsDNA and who did not develop high T3 and T4 levels showed normal thyroid gland size and morphology (FIG. 10C and 10D). Representative pictures of thyroid glands are shown in FIG. 10. In panel A, we show the picture of a thyroid gland from a DNA-transfected hTSHR-DAP.3 immunized mouse who developed hyperthyroidism in Table 1. In panel B, the histology of the thyroid gland shown in Panel A (magnification: 40×) is presented. In panel C we show the thyroid gland of a mouse immunized with hTSHR DAP.3 cells which were not transfected with dsDNA. In panel D we show the histology of the thyroid gland shown in C (magnification: 40×). Thyroid glands were fixed in formalin for histological examination after hematoxylin-eosin staining. Note that the magnification is same for B and D.

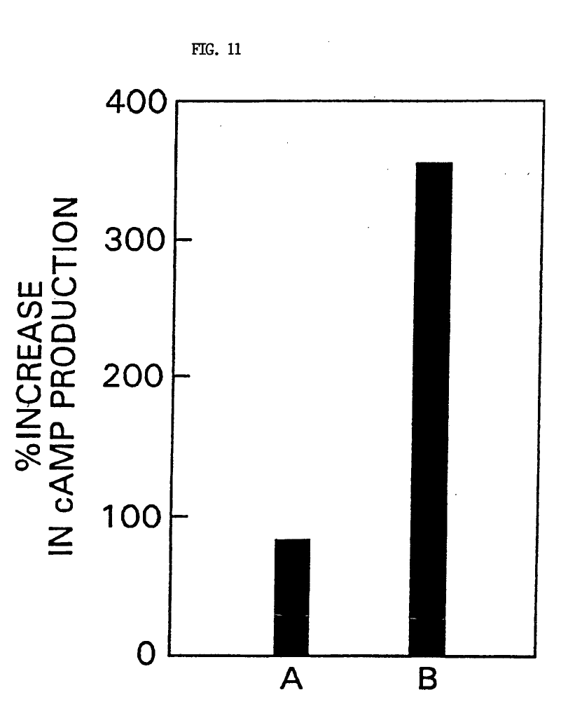

Protein A-purified IgG from mice immunized with dsDNA-transfected hTSHR DAP.3 cells, and who developed high serum T4 and T3 levels, had significant levels of stimulating thyrotropin receptor autoantibody (TSHRAb) activity in cAMP assays, measured using CHO cells transfected with hTSHR (W. B. Kim, et al., J. Clin Endocrinol. Metab. 81: 1758-1767 (1996)) (FIG. 11, group B). In contrast, IgG from mice immunized with hTSHR-transfected DAP.3 cells which had not been transfected dsDNA but which had been lipofectamine treated, exhibited no stimulating TSHRAb activity (FIG. 11; group A). Stimulating TSHRAb activity was measured using hTSHR-transfected CHO cells and IgG, purified on a protein A-Sepharose column, from the serum of the mice in Table 1. The data presented were obtained from one hyperthyroid mouse (A) and one normal mouse (B) but were duplicated in all hyperthyroid or normal mice in Table 1.

The presence of stimulating TSHRAb activity in the IgG fraction (FIG. 11) and elevated thyroid hormone levels (Table 1) were directly correlated in all mice. The development of increased thyroid hormone levels correlated, therefore, with the development of stimulating antibodies directed against the TSHR not TBII activity.

In previous studies (N. Shimojo, et al., Proc. Natl. Acad. Sci. U.S.A. 93: 11074-11079 (1996); K.-I Yamaguchi, et al., J. Clin. Endocrinol. Metab. 82: 4266-4269 (1997); S. Kikuoka, et al., Endocrinology 139: 1891-1898 (1998)), about 20-25% of all mice immunized with fibroblast containing the hTSHR in the context of aberrant Class II expression developed features characteristic of Graves' disease: stimulating TSHRAbs, increased thyroid hormone levels, TBIIs directed at the TSHR, and enlarged thyroids with thyrocyte hypercellularity and thyrocyte intrusion into the follicular lumen. The incidence is statistically significant, p<0.05, by comparison to controls, and was replicated in multiple experiments. Most of the remaining mice developed TSHRAbs characteristic of Graves' TBIIs, i.e. having the ability to inhibit TSH-increased cAMP levels; this incidence is statistically significant by comparison to the control group at p<0.01. These features were not duplicated in mice immunized with control fibroblasts expressing the TSHR alone or expressing aberrant MHC Class II alone.

Previous studies in which mice immunized with the soluble extracellular domain of TSHR, either baculovirus-produced and glycosylated or prokaryotic in origin, failed in their intent to produce a model of Graves'-disease amenable to study the pathophysiology of this disease process (G. S. Seetharmamaiah, et al., Autoimmunity 14: 315-320 (1993); S. Costagliola, et al., J. Mol. Endocrinol. 13: 11-21 (1994); S. Costaglioloa, et al., Biochem. Biophys. Res. Commun. 199: 1027-1034 (1994); S. Costagliola, et al., Endocrinology 135: 2150-2159 (1994); A. Marion, et al., Cell. Immunol. 158: 329-341 (1994); N. M. Wagle, et al., Autoimmunity 18: 103-108 (1994); G. Carayanniotis, et al., Clin. Exp. Immunol. 99: 294-302 (1995); G. S. Seetharamaiah, et al., Endocrinology 136: 2817-2824 (1995); N. M. Wagle, et al., Endocrinology 136: 3461-3469 (1995); H. Vlase, et al., Endocrinology 136: 4415-4423 (1995)). Thus, even if TBII activity was detected in these studies, in most cases the activity was not shown to reflect the existence of an antibody against the TSHR in TSHR transfected cell (G. S. Seetharamaiah et al., Autoimmunity 14: 315-320 (1993); S. Costagliola, et al., J. Mol. Endocrinol. 13: 11-21 (1994); S. Costagliola, et al., Biochem. Biophys. Res. Commun. 199: 1027-1034 (1994); S. Costagliola, et al., Endocrinology 135: 2150-2159 (1994); A. Marion, et al., Cell. Immunol. 158: 329-341 (1994); N. M. Wagle, et al., Autoimmunity 18: 103-108 (1994); G. Carayanniotis, et al., Clin. Exp. Immunol. 99: 294-302 (1995); G. S. Seetheramaiah, et al., Endocrinology 136: 2817-2824 (1995); N. M. Wagle, et al., Endocrinology 136: 3461-2469 (1995); H. Vlase, et al., Endocrinology 136: 4415-4423 (1995)). Similarly, there were no histological findings of thyrocyte hypertrophy together with increased serum thyroid hormone levels in any of these studies, only thyroiditis in some. Most important, in no case were stimulating TSHRAbs produced which could cause hyperthyroidism, thyroid enlargement, or thyrocyte hypercellularity. The past results (N. Shimojo, et al., Proc. Natl. Acad. Sci. U.S.A. 93: 11074-11079 (1996); K.-I Yamaguchi, et al., J. Clin. Endocrinol. Metab. 82: 4266-4269 (1997); S. Kikuoka, et al., Endocrinology 139: 1891-1898 (1998); thus show that a functional TSHR within the cell membrane, if presented to the immune system in the context of an aberrantly expressed MHC antigen, can induce an immune disease with major features of GD: stimulating TSHRAbs, TSHRAbs which inhibit TSH binding and activity, increased thyroid hormone levels, thyroid enlargement, and thyrocyte hypercellularity.

Viruses, bacteria, environmental insults, and/or tissue injury can cause autoimmunity, including diabetes and autoimmune thyroid disease (Y. Tomer and T. Davies, Endocr. Rev. 14: 107-121 (1993); M. Saji, et al., J. Clin. Endocrinol. Metab. 75: 871-878 (1992); L.D. Kohn, et al., Intern. Rev. Immunol. 9: 135-165 (1992); E. Mozes, et al., Science 261: 91-93 (1993); D. S. Singer, et al., J. Immunol. 153: 873-880 (1994); L. D. Kohn, et al., in Thyroid Immunity, D. Rayner and B. Champion (Eds.), R. G. Landes Biomedical Publishers, Texas, pp. 115-170 (1995)). Increasing evidence exists that this is caused by a target tissue effect not an immune cell defect, molecular mimicry, nor cytokine stimulation, which appears to be a secondary phenomenon (M. Saji, et al., J. Clin. Endocrinol. Metab. 75: 871-878 (1992); L. D. Kohn, et al., Intern. Rev. Immunol. 9:135-165 (1992); E. Mozes, et al., Science 261: 91-93 (1993); D. S. Singer, et al., J. Immunol. 153:873-880 (1994); L. D. Kohn, et al., in Thyroid Immunity, D. Rayner and B. Champion (Eds.), R. G. Landes Biomedical Publishers, Texas, pp. 115-170 (1995); F. Schuppert, et al., Thyroid 7: 837-842 (1997); M. S. Horowitz, et al., Nature Medicine 4: 781-785 (1998); H. Wekerle, Nature Medicine 4: 770-771 (1998); C. Benoist & D. Mathis, Nature 394: 227-228 (1998)). We now show that ds DNA can increase MHC class I and class II antigen expression and increase expression of genes encoding proteins important for antigen presentation in fibroblasts. We show that immunization of dsDNA-transfected fibroblasts which also contain the hTSHR results in the development of exactly the same Graves' disease-like syndrome as hTSHR transfected RT4.15HP fibroblasts genetically engineered to aberrantly express MHC class II genes (N. Shimojo, et al., Proc. Natl. Acad. Sci. U.S.A. 93: 11074-11079 (1996); K.-I. Yamaguchi, et al., J. Clin. Endocrinol. Metab. 82: 4266-4269 (1997); S. Kikuoka, et al., Endocrinology 139: 1891-1898 (1998)). Thus, we establish that transfection of dsDNA not only mimics the action of viral infection and viral DNA (Example 1), it can be the intermediate event in developing an autoimmune disease.

In the original studies (N. Shimojo, et al., Proc. Natl. Acad. Sci. U.S.A. 93: 11074-11079 (1996); K.-I. Yamaguchi, et al., J. Clin. Endocrinol. Metab. 82: 4266-4269 (1997); S. Kikuoka, et al., Endocrinology 139: 1891-1898 (1998)), the mechanism by which the antigen was processed by the normal immune cells, for example, the source of the costimulatory molecules in the development of this immune response was unclear. Certainly there were studies (T. M. Kundig, et al., Science 268: 1343-1347 (1995)) which showed that immunization of mice with fibroblasts transfected with viral protein could induce a CTL response in the absence of costimulatory molecules on the immunizing fibroblasts, suggesting costimulatory signals are host derived. In the present experiments this problem is obviated by the demonstration (see for example, Example 2) that the B7.1 costimulatory molecule is increased on the fibroblasts by dsDNA. In short, in these experiments, there is no question that dsDNA transfection provides the full array of antigen presenting molecules needed for the autoimmune response, as well as increased MHC class I and aberrant class II.

Since the immunized mice have a normal complement of T and B cells, the mechanism by which this disease develops must involve the breaking of normal immune tolerance. Thus, these data support the conclusion that a viral or environmental insult of the target tissue, in this case the thyroid, can lead to autoimmune disease independent of a viral action on the immune cells. This is not molecular mimicry. In short, these data are consistent with the model that any ds nucleic acid fragment, introduced in the cytoplasm by infection or leakage of self DNA, can directly induce MHC expression, and, concomitantly, increase or activate other essential factors important for antigen presentation. This can turn normal cells into antigen presenting cells with abnormally expressed MHC genes and thereby enable them to present auto- or foreign-antigens to our immune cell repertoire. This may be induced by viral DNA, ds viral RNA produced during the replication of RNA viruses, or perhaps viral- or environmentally-induced tissue damage. We suggest this is a plausible mechanism to explain the evidence that viruses trigger autoimmune disease by bystander activation of T cells not molecular mimicry (M. S. Horowitz, et al., Nature Medicine 4: 781-785 (1998); H. Wekerle, Nature Medicine 4: 770-771 (1998); C. Benoist & D. Mathis, Nature 394: 227-228 (1998)).

The data are consistent with the evidence indicating that the virus infection of the target tissue presents self-antigens to activate T cells in the normal repertoire (M. S. Horowitz, et al., Nature Medicine 4: 781-785 (1998); H. Wekerle, Nature Medicine 4: 770-771 (1998); C. Benoist & D. Mathis, Nature 394: 227-228 (1998); and that these induce the cytokine (IL-18/IL-12/KIFN) cascade which furthers the autoimmune process (M. S. Horowitz, et al., Nature Medicine 4: 781-785 (1998); H. Wekerle, Nature Medicine 4: 770-771 (1998); C. Benoist & D. Mathis, Nature 394: 227-228 (1998)). An additive or, perhaps, even synergistic increase in MHC gene expression in the target tissue, induced by the initial dsDNA insult and then the reactive immune cell production of cytokines and KIFN, may convert a normal protective process to an autoimmune process.

There are several possible explanations why only about 20-30% of mice develop stimulating TSHRAbs which caused hyperthyroidism when immunized with hTSHR RT4.15HP cells or DNA-transfected hTSHR DAP.3 cells (Table 1) (N. Shimojo, et al., Proc. Natl. Acad. Sci. U.S.A. 93: 11074-11079 (1996); K.-I. Yamaguchi, et al., J. Clin. Endocrinol. Metab. 82: 4266-4269 (1997); S. Kikuoka, et al., Endocrinology 139: 1891-1898 (1998)), whereas most mice produced anti-TSHR antibodies detected by the TBII assay. Different mechanisms to produce the two antibodies certainly exist (N. Shimojo, et al., Proc. Natl. Acad. Sci. U.S.A. 93: 11074-11079 (1996); K.-I. Yamaguchi, et aL, J. Clin. Endocrinol. Metab. 82: 4266-4269 (1997); S. Kikuoka, et al., Endocrinology 139: 1891-1898 (1998)). Nevertheless, these experiments were short term, with a total of 6 immunizations 2 weeks apart before termination of the experiment. Longer time periods of observation might result in more animals with stimulating TSHRAbs and hyperthyroidism. An alternative or related possibility may lie in the quantitative aspects of MHC gene expression or the quantitative level of expression in combination with overexpressed genes important for antigen presentation. Thus, 75% of mice immunized with dsDNA-transfected hTSHR RT4.15HP cells developed hyperthyroidism and the Graves′-like syndrome in the same time frame. These cells have a quantitatively increased level of aberrant MHC class II (FIG. 8) plus the increase in or activation of proteins important for antigen presentation. Thus, as predicted (N. Shimojo, et al., Proc. Natl. Acad. Sci. U.S.A. 93: 11074-11079 (1996); K.-I. Yamaguchi, et al., J. Clin. Endocrinol. Metab. 82: 4266-4269 (1997); S. Kikuoka, et al., Endocrinology 139: 1891-1898 (1998)), greater levels of class II expression in the fibroblasts may increase the frequency of stimulating TSHRAb-positive mice. Additionally, increased MHC class I expression and expression of antigen presenting molecules, in addition to aberrant class II, enhances the frequency of stimulating TSHRAb positive mice.

Studies of 5′-flanking region cis regulatory elements of the class I and TSHR genes, together with their respective trans factors, suggest the importance of abnormal class I molecules in the expression of GD or other forms of autoimmunity (L. D. Kohn, et al., Intern. Rev. Immunol. 9: 135-165 (1992);.E. Mozes, et al., Science 261: 91-93 (1993); D. S. Singer, et al., J. Immunol. 153: 873-880 (1994); L. D. Kohn, et al., in Thyroid Immunity, D. Rayner and B. Champion (Eds.), R. G. Landes Biomedical Publishers, Texas, pp. 115-170 (1995)). These data additionally indicate there are common elements in the class I and class II molecules (L. D. Kohn, et al., in Thyroid Immunity, D. Rayner and B. Champion (Eds.), R. G. Landes Biomedical Publishers, Texas, pp. 115-170 (1995)). The present findings using dsDNA-transfected hTSHR RT4.15HP cells support the conclusion that both class I and class II molecules are important in the development of GD.

In summary, the present data offer the novel result that ds nucleic acids, by increasing MHC gene expression and the expression of antigen presenting genes can cause a cell with a finctional TSHR to induce an autoimmune response, mediated by the normal T and B cell population. The disease mimics the major features of anti-TSHR receptor autoimmunity expressed in Graves' disease and supports the thesis that a primary viral or environmental insult of the target tissue, using this pathway, can induce autoimmune disease (M. S. Horowitz, et al., Nature Medicine 4: 781-785 (1998); H. Wekerle, Nature Medicine 4: 770-771 (1998); C. Benoist & D. Mathis, Nature 394: 227-228 (1998)).

Based on the data in this Example and in Example 4, this autoimmunity model offers, therefore, an in vivo means to test drugs active in vitro to suppress the ds nucleic acid induced increases in MHC gene expression and increases in the expression of antigen presenting molecules.

### Example 6

THE ABILITY OF DOUBLE STRAND POLYNUCLEOTIDES TO ENHANCE EXPRESSION OF THE 90K TUMOR-ASSOCIATED IMMUNOSTIMULATOR DIRECTLY LINKS THIS PHENOMENON TO HOST MECHANISMS TO DEFEND AGAINST ONCOGENE TRANSFORMATION (TUMORS) AND AQUIRED IMMUNODEFICIENCY DISEASE (AIDS)

The ability of double strand polynucleotides to increase the 90K tumor-associated immunostimulator, when transfected into mammalian cells, was first noted in Example 2, FIG. 3. The 90K tumor-associated immunostimulator has an important role in host defense mechanisms directed at tumors and AIDS. The present studies were aimed at understanding the role of ds nucleic acids in increasing the 90K tumor-associated immunostimulator and its relationship to the action of ds nucleic acids in autoimmunity, neoplastic disease, and AIDS.

Studies using monoclonal antibodies directed at tumor-related components in the culture fluid of human breast cancer cells led to the identification of a secreted, approximately 90 kDa protein, designated 90K, in a high proportion of breast cancers (S. Iacobelli, et al., Cancer. Res. 46: 3005-3010 (1986)). Subsequent studies showed that this 90K tumor-associated protein was highly glycosylated and was present in the sera of normal individuals, but existed at much higher levels in the sera of patients with multiple forms of cancer (S. Iacobelli, et al., Breast Cancer Res. Treat. 11: 19-30 (1998); G. Scambia, et al., Anticancer Res. 8: 761-764 (1998); S. Iacobelli, et al., Br. J. Cancer 69: 172-176 (1994); O. Fusco, et al., Int. J. Cancer 79: 23-26)). High levels of the 90K protein were also found in the serum of patients infected by the human immunodeficiency virus (HIV), even in the apparent absence of neoplastic complications (C. Natoli, et al., J. Infect. Dis. 164: 616-617 (1991); S. Iacobelli, et al., J. Infect. Dis. 164: 819 (1991); C. Natoli, et al., J. AIDS 6: 370-375 (1993); N. Briggs, AIDS Res. Hum. Retroviruses 9: 811-816 (1993); S. lacobelli, et al., J. AIDS 10: 450-456 (1995)).

A molecular cloning study (A. Ullrich, et al., J. Biol. Chem. 269: 18401-18407 (1994)) reveled that 90K is a member of the scavenger receptor cysteine-rich (SRCR) domain family and is identical to Mac-2 binding protein (mac-2 bp), the dominant ligand for macrophage-associated S-type lectin, Mac-2 (also know as galectin-3), which is expressed at significantly higher levels in activated macrophages and may be involved in events as diverse as cell migration, immune modulation, and cancer metastasis (M. M. Lotz, et al., Proc. Natl. Acad. Sci. USA 90: 3466-3470 (1993)). 90K is also highly homologous to the murine adherent macrophage (MAMA) protein, a membrane glycoprotein that is induced by macrophage adhesion (Y. Chicheportiche and P. Vassalli, J. Biol. Chem. 269: 5512-5517 (1994)).

Functional date indicate that the over expression of human 90K in mouse mammary carcinoma cells lines dramatically reduced their tumorigenicity in nude mice, locally as well as systemically (B. Gall, et al., Cancer Res. 55: 3223-3227 (1995)). Increased expression of 90K led also to induction of intracellular adhesion molecule-1 (ICAM-1)) in the tumor endothelium. This was consistent with its know relation to Mac-2 and MAMA. Additional functional data suggested that the 90K protein participated in activation of the host immune system, resulting in a more effective anti-tumor response. Thus, recombinant 90K has been shown (A. Ullrich, et al., J. Biol, Chem. 269: 18401-18407 (1994)) to enhance the in vitro generation of cytotoxic effector cells (NK an LAK) from peripheral blood mononuclear cells (PBMC) and to increase IL-2 production by PBMC stimulated with suboptimal concentrations of concanavalin A (ConA). Also, 90K protein purified from human serum can enhance expression of major histocompatibility (MHC) Class I molecules in human breast cancer cells. (C. Natoli, et al., Biochem. Biophys. Res Commum. 225: 617-620 (1996)). Third, observations in cancer patients and in vitro have documented that 90K is induced by I- and K-interferon (IFN) and by tumor necrosis factor-I, (TNF-I) (S. Iacobelli, et al., Int. J. Cancer 42: 182-184 (1998); C. Natoli, et al., Brit. J. Cancer 67: 564-567 (1993); C. Marth, et al., Int. J. Cancer 59: 808-813 (1994)). These last data led to the proposal that 90K has the finction of an immune stimulatory molecule, and was designated the 90K tumor-associated immunostimulator.

To better understand the biological function and possible role of 90K in the context of immune host defense, we examined the expression of the protein in a normally functioning noncancerous model cell system. We cloned the cDNA and 5′-flanking region of the 90K gene from a FRTL-5 rat thyroid cell library and studied its expression in these thyrocytes. FRTL-5 cells are a continuously cultured line which have no attributes of tumor cells, exhibit thyrotropin (TSH) and insulin/insulin-like growth factor-I-dependent growth and function, and mimic normal thyrocytes in vivo in almost all respects (F. S. Ambesi-Impiombato, U.S. Pat. No. 4,608,341 (1986); L. D. Kohn, et al., U.S. Pat. No. 4,609,622 (1986); F. S. Ambesi-Impiombato and H. Perrild, FRTL-5 Today Int. Congress Series 818, Excerpta Medica, Amsterdam, The Netherlands, pp. 1-286 (1989); L. D. Kohn, et al., in Thyroid Immunity, D. Rayner and B. Champion (Eds.) R. G. Landes Biomedical Pub., Austin and Georgetown, Tex. pp 115-170 (1995); L. D. Kohn, et al., Vitamins and Hormones 50: 287-384 (1995)). We showed that expression of the 90K immunostimulator in FTRL-5 cells is under TSH/insulin, as well as KIFN control. Of interest, we showed that a viral promoter, transfected into FTRL-5 thyroid cells, such as that of the cytomegalic virus (CMV), coincidentally increased 90K tumor-associated immunostimulator and major histocompatibility (MHC) Class I RNA levels in the absence of changes in [symbol] -actin and several transcription factors known to regulate MHC Class I activity. The data suggested that the 90K tumor-associated immunostimulator, which is under hormonal control in a normally functioning thyrocyte, might help regulate MHC Class I levels in response to viral infections.

It has been shown that polyI-polyC, a polynucleotide mimicking the double stranded RNA produced by viruses, as well as KIFN, could increase 90K gene expression in cells transfected with the Class I mouse promoter (C. Brakebush, et al., J. Biol. Chem. 272: 3674-3682 (1997)). We have shown that polyl-polyC behaves like ds DNA not KIFN, with the exception that it increases [symbol] -IFN production in the target (Example 2). This led us to speculate that ds nucleic acids would increase expression of the 90K tumor-associated immunostimulator in FRTL-5 cells, that it might be an intermediate in the signal transduction process leading to MHC Class I gene expression, and that it might be over expressed in thyroid tumors as a normal defense mechanism to inhibit their growth and increase immune cell targeting, thereby causing apoptosis or tumor cell killing. The following experiments were designed to evaluate these possibilities.

Experimental Protocol

Cells—Rat FRTL-5 thyroid cells were a fresh subclone (F1) with all properties described (F. S. Ambesi-Impiombato, U.S. Pat. No. 4,608,341 (1986): L. D. Kohn, et al., U.S. Pat. No. 4,609,622 (1986); F. S. Ambesi-Impiombato and H. Perrild, FRTL-5 Today, Int Congress Series 818, Excerpta Medica, Amsterdam, The Netherlands, pp. 1-286 (1989); L. D. Kohn, et al., in Thyroid Immunity, D. Rayner and B. Champion (Eds.), R. G. Landes Biomedical Pub., Austin and Georgetown, Tex. pp. 115-170 (1995); L. D. Kohn, et al., Vitamins and Hormones 50: 287-384 (1995)). They were grown in 6H medium consisting of Coon's modified F12 medium, 5% heat-treated, mycoplasma-free, calf serum, 1 mM nonessential amino acids, and a six hormone mixture; bovine TSH (1×10⁻¹⁰M), insulin (10 Tg/ml), cortisol (0.4 ng/ml), transferrin (5 Tg/ml), glycl-L-histidyl-L-lysine acetate (10Tg/ml), and somatostatin (10 ng/ml). Cells, were fed every 2-3 days and passaged every 7-10 days.

Library Screening, DNA Sequencing, and Sequence Analysis—To isolate the rat 90K cDNA, a previously described Σgt11 rat cDNA library (T. Akamizu, et al., Proc. Natl. Acad. Sci U.S.A. 87: 5677-5681 (1990)), constructed using FRTL-5 cell poly(A+) RNA, was screened by plaque hybridization with ³²P-labeled human 90 K cDNA. Hybridization was preformed at 68° C.; washes were performed at room temperature and at 37° C. DNA fragments from the screening were subcloned into pGEM7zf(+) (Promega, Madison, Wis.) and sequenced, using the dideoxynucleotide chain termination method (F. Sanger F., et al., Proc Natl., Acad. Sci. U.S.A. 74: 5463-5467 (1997)) and T7, SP6, or site-specific synthetic oligonucleotide primers. Sequence alignments and comparisons were performed using Gene Works IntelliGenetics, Inc., Mountain View, Calif.).

Recombinant Protein Production in E. coli—Recombinant protein was produced using the pET system (Novagen, Madison, Wis.). The 90K cDNA insert was ligated to the EcoRi site of the expression vector, pET-30(+), allowing the His-Tag sequence to be linked to its N-terminus. After transforming using E. Coli BL21 (DE3), a single colony was inoculated in 50 ml LB medium containing 30 Tg/ml kanamycin and incubated with shaking at 37° C. At 0.6 OD600, isopropyl- [symbol] -d-thiogalactopryanoside (IPTG) was added to 1 mM. After 2 hours, the induced cells were collected by centrifugation (5,000×g, 5 min, 4E C), resuspended in 4 ml ice-cold binding buffer (5 mM imidazole, 0.5 M NaCl, 20 mM Tris-HCl, pH 7.9), then sonicated until no longer viscous. Cell extracts were centrifuged (39,000×g, 20 min, 4° C.); the supernatant was applied to His-Bind columns containing resin-immobilized Ni²⁺; and the columns were washed with 25 ml binding buffer. Unbound proteins were removed with 15 ml elute buffer containing imidazole. The His-Bind column contained 5 ml resin and was washed, sequentially, with 7.5 ml deionized water, 12.5 ml charge buffer (50 mm NiSO₄) and 12.5 ml binding buffer. After Addition of a ⅓ rd volume of Strip Buffer, the eluted fraction was dialyzed against 20 mM HEPES-KOH, pH 7.9, 100 mM KCL, 0.1 mM EDTA, 20% glycerol, 0.5 mM dithiothreitol (DDT), 0.5 mM phenylmethylsulfonyl fluoride (PMSF), 2 Tg/ml pepstatin A, then concentrated in a Centricon 10 (Amicon, Beverly, Mass.) for use in binding experiments.

RNA isolation and Northern Blot Analysis—Cells were treated with 100 U/ml rat KIFN (P. L. Baldcucci-Silano, et al., Endocrinology 139: 2300-2313 (1998); V. Montani, et al., Endocrinology 139: 290-302 (1998); or transfected with 5 Tg ds DNA or ds RNA using Lipofectamine Plus (GIBCO BRL, Gaithersburg, Md.) as described in Examples 1 through 3. Total RNA was prepared and Northern analysis performed using nitrocellulose membranes (Nytran Plus, Schleicher & Schuell) as described (O. Isozaki, et al., Mol. Endocrinol. 3: 1681-1692 (1989); M. Saji, et al., J. Clin. Endocrinol. Metab. 75: 871-878 (1992); P. L. Balducci-Silano, et al., Endocrinology 139: 2300-2313 (1989); V. Montani, et al., Endocrinology 139: 290-302 (1989); S.-I.Taniguchi, et al., Mol. Endocrinol. 12: 19-33 (1998)). Filters were sequentially hybridized with the rat 90K, MHC Class I, MHC Class II, and GAPDH probes. Radiolabeling of all probes and hybridization (1.0×10⁶ cpm/ml) were as described (O. Isozaki, et al., Mol. Endocrinol. 3: 1681-1692 (1989); M. Saji, et al., J. Clin. Endocrinol. Metab 75: 871-878 (1992); P. L. Balducci-Silano, et al., Endocrinology 139: 2300-2313 (1998); V. Montani, et al., Endocrinology 139: 290-302 (1998); S.-I. Taniguchi, et al., Mol. Endocrinol. 12: 19-33 (1998)). The rat 90K cDNA was the full length clone isolated in the screening procedure, the MHC Class I probe and Class II probes were those described in Examples 1 through 5 and the following references (M. Saji, et al., J. Clin. Endocrinol. Metab. 75: 871-878 (1992); P. L. Balducci-Silano, et al., Endocrinol. 12: 19-33 (1998)). The glyceraldehyde phosphate dehydrogenase (GAPDH) probe was cut from a pTRI-GAPDH-Rat template (Ambion, Tex.).

Peptide Synthesis and Antibody Production—Based on the deduced amino acid sequence, we chose 2 peptides, 17 amino acids each, which were identified as immunogenic with the aid of the Gene Works program. Peptide #1 represented amino acids 530-546; peptide #2 represented amino acids 438-454. Peptides were synthesized by Genemed Biotechnologies (San Francisco, Calif.) and were utilized to immunize rabbits after being linked to Keyhole limpet hemocyanin (KLH) (N. Green, et al., Cell 28: 477-487 (1982)). The rabbit antibody used herein reacts with peptide #1 but not peptide #2 and can detect Western blotted, purified 90K recombinant protein.

Immunobloting—Samples were transferred to nitrocellulose membranes by manual blotting. Protein was identified after antibody binding using the ECL method (Amersham Life Science, Cleveland, Ohio.)

Results

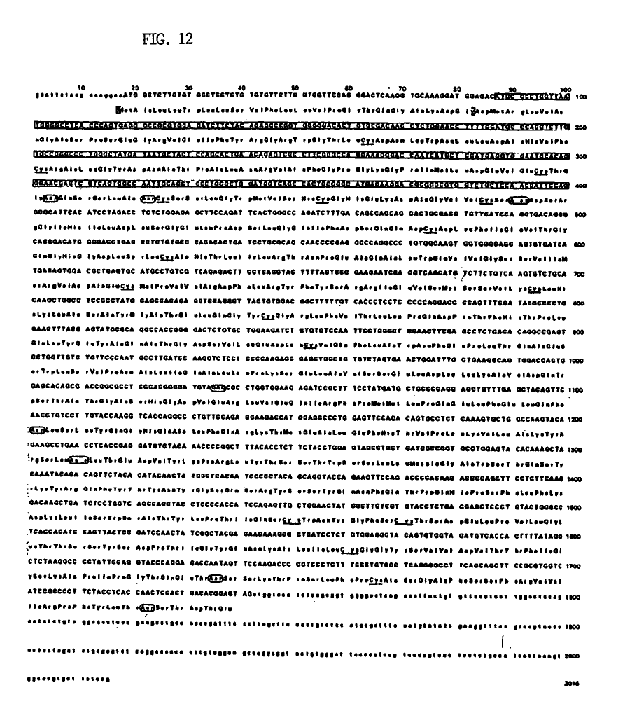

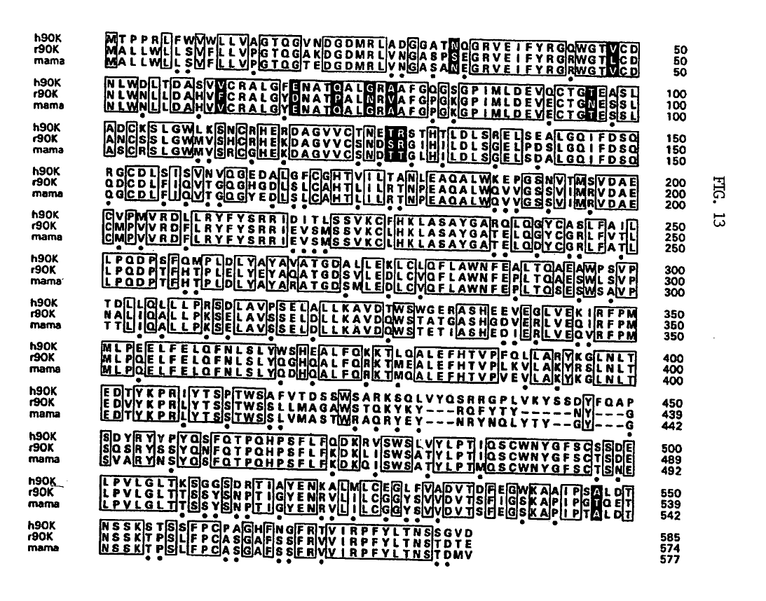

The rat 90K cDNA extends 2016 nucleotides (FIG. 12); the open reading frame starts from the ATG initiation codon at nucleotide 18 and ends at the TAG termination codon at position 1740. It encodes a protein of 574 amino acids with a calculated molecular weight 67,490; there are 7 potential glycosylation sites and 16 cysteine residues. The first 18 amino acids have the characteristics of a signal peptide sequence (L. J. Dangott, et al., Proc. Natl. Acad. Sci. U.S.A. 86: 2128-2132 (1989)). Analysis of the amino acid sequence revealed a high degree of identity with both the murine adherent macrophage (MAMA) and human Mac-2 (human 90K) binding proteins. (FIG. 13). All cysteine residues were conserved, as was the region coding for the scavenger receptor Cysteine-rich (SRCR) domain, amino acids 24-128. This domain is also found in the speract receptor (A. Aruffo, et al., J. Biol. Chem. 272: 3674-3682 (1997)). The three proteins diverge in a region spanning residues 431 through 449 of human 90K (FIG. 13). In sum, the rat 90K protein is highly homologous with the human 90K tumor-associated immunostimulator and study of its biological properties in the FRTL-5 cells should be a reasonable index of the properties of human 90K.

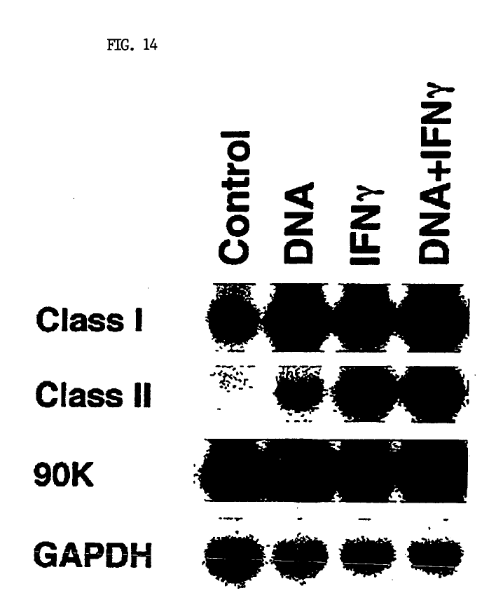

Northern analysis, performed on FRTL-5 cells treated with 100 U/ml KIFN, transfected with 5 Tg dsDNA, or both, after being maintained for 7 days in medium with TSH plus 5% calf serum, revealed that 90K RNA was constitutively expressed in FRTL-5 cells but that its expression was markedly enhanced by dsDNA (FIG. 14).

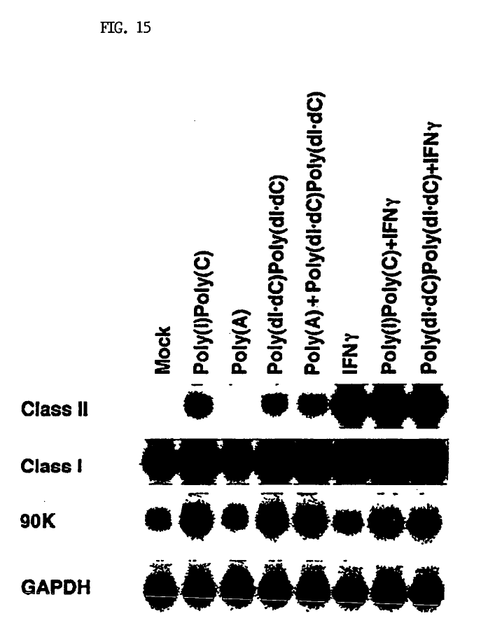

Examining the effects of different types of ds nucleic acids (FIG. 15), we found that increase was effected by ds RNA as well as dsDNA, but not the single strand nucleic acids as in Examples 1 and 2. Again, the KIFN effect was weaker than not only dsDNA but also dsRNA.

Importantly, there was a close correlation of the increase in 90K RNA with those of MHC Class I but not MHC Class II levels, whereas, KIFN increases Class II more than Class I levels (Example 1, FIG. 1C and 1D). This suggests that the observations that polyI-polyC, a polynucleotide mimicking the double stranded RNA of viruses, could increase 90K gene expression in cells transfected with the mouse promoter (C.

Brakebush, et al., J. Biol. Chem. 272: 3674-3682 (1997)) was not an action mimicking KIFN, but rather was an effect of the ds nucleic acids.

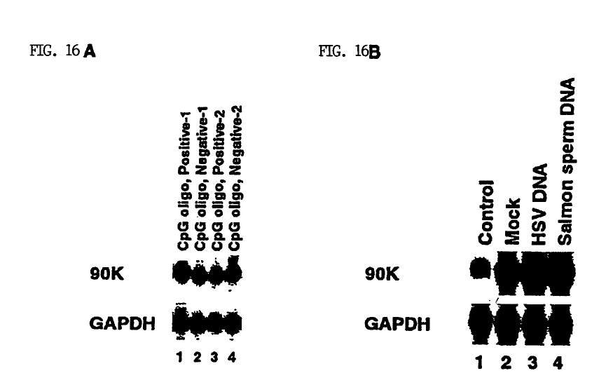

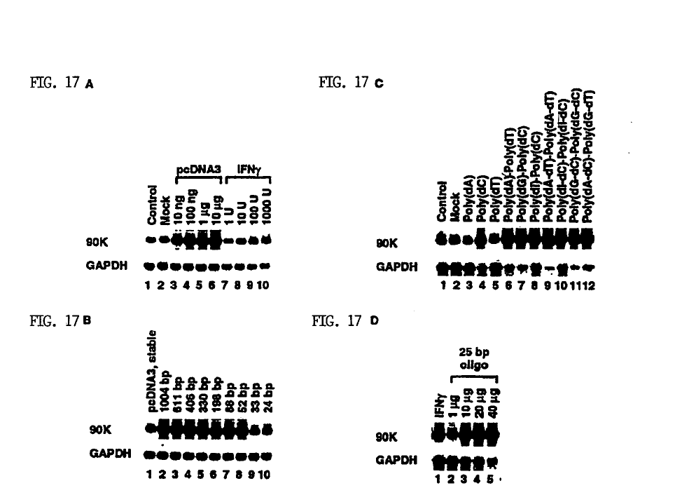

The increase in 90K RNA levels was evident whether CpG residues were methylated or not (FIG. 16A) and were seen using either viral DNA or salmon sperm DNA (FIG. 16B), as reported for ds nucleic acids (Example 2). The ability of ds nucleic acids to increase 90K RNA levels mimicked their ability to increase MHC Class I levels as a function of dsDNA concentration (FIG. 17A), as a function of nucleotide length (FIG. 17B), and as a function of all oligonucleotides which were tested (FIG. 17C and 17D).

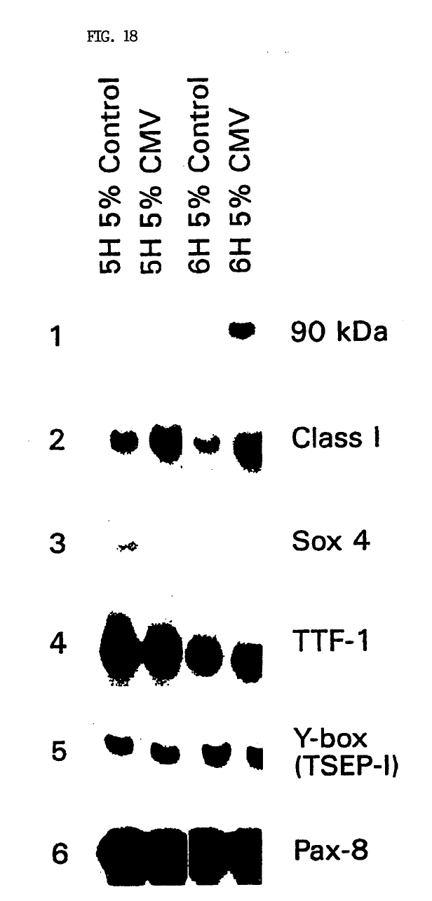

Transfection of viruses and their promoters into cells is well known to increase MHC Class I gene expression and antigen levels (D. S. Singer & J. E. Maguire, CRC Crit. Rev. Immunol. 10: 235-257 (1990); J. P.-Y. Ting & A. S. Baldwin, Curr. Opin. Immunol. 5: 8-16 (1993)). In accord with this, it was not surprising that transfection of the cytomegalic virus (CMV) promoter, pRcCMV, into FRTL-5 thyroid cells significantly increased class I RNA levels (FIG. 18, Row 2). More interestingly, however, we noted a coincident increase in 90K RNA levels (FIG. 18, Row 1), particularly in TSH treated (6H) cells. Similar results were obtained with plasmids containing SV40 and HIV promoters (data not shown). This was highly, specific, since no concurrent shifts in [symbol] -actin (data not shown), as well as Sox-4, TTF-1 thyroid Y-box (TSEP-1), or Pax-8 RNA levels (FIG. 18, Rows 3-6), all of which are transcription factors involved in TSH regulation of MHC Class I gene expression (L. D. Kohn, et al., in Thyroid Immunity, D. Rayner and B. Champion, (Eds.) R. G. Landes Biomedical Pub., Austin and Georgetown, Texas pp. 115-170 (1995); K. Suzuki, et al., Thyroid 5 (Suppl 1): S1 (1995); C. Giuliani, et al., J. Biol. Chem. 270: 11453-11462 (195); M. Saji, et al., J. Biol. Chem. 272: 20096-20107 (1997): M. Ohmori, et al., Thyroid 5 (Supp 1): 37 (1996)).

The ability of pRcCMV to increase 90K RNA levels was transcriptional, as evidenced in nuclear run-on assays, where the 90K increase was 6.4 fold higher relative to [symbol] -actin and Y-box, which did not change, and 10.7-fold greater than TTF-1, which decreased 2-fold.

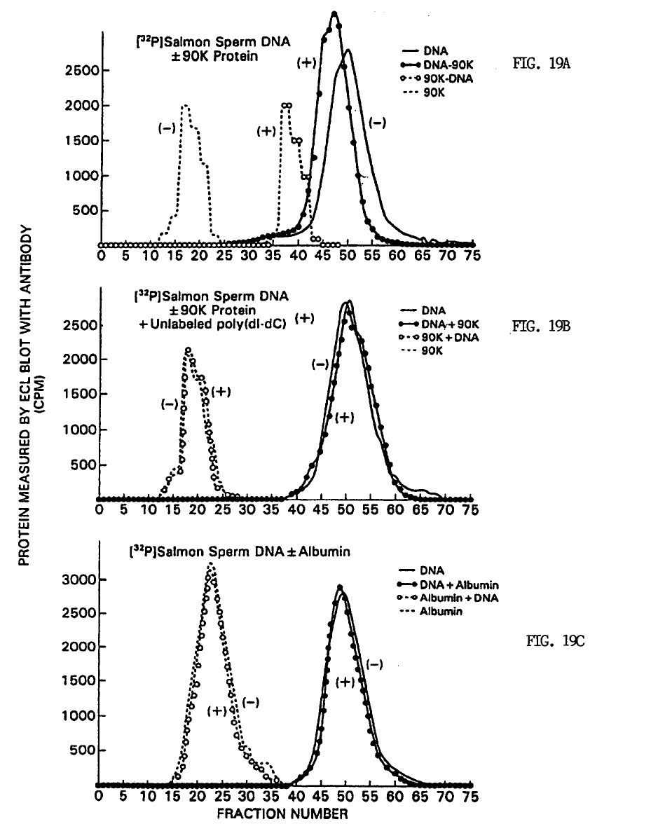

The close association of the Class I and 90 K RNA increases plus the ability of 90K protein purified from human serum to enhance expression of MHC Class I molecules in human breast cancer cells (C. Natoli, et al., Biochem. Biophys. Res. Commun. 225: 617-620 (1996)) led us to consider that 90K protein might also be an intermediate in the process of transcriptional regulation by binding the dsDNA. This possibility is not unrealistic since short DNA sequences in the cytoplasm of Ehrlich ascites tumor cells are highly associated with proteins (R. Hegger & H. Abken, Physiol. Chem. Phys. Med. NMR 27: 321-328 (1995)). We examined this possibility in the following experiment (FIG. 19). Sheared salmon sperm DNA was ³²P-radiolabeled using procedures for radiolabeling nucleotide probes. The ³²P-radiolabeled DNA, 500,000 cpm, was passed on a G-100 Sephadex column as was 50 Tg recombinant 90K, protein (FIG. 19A). The recombinant protein was assayed by blotting fractions on nitrocellulose and detecting it with an antibody to peptide #1 of the 90K protein, amino acids 530-546. The radiolabeled DNA and 90K recombinant protein were then incubated together for 20 min and passed over the same column. The 90K protein now migrated near the end of the collected fractions, overlapping a region of the radiolabeled DNA, whose peak shifted to earlier fractions. These data indicated that the dsDNA was able to bind 90K not only induce its synthesis. This conclusion was strengthened by adding 250 Tg of the dsDNA oligonucleotide, poly (dI-dC) to the incubations (FIG. 19B); poly(dI-dC) was used in the transfection experiments (Example 2). The presence of the unlabeled oligonucleotide inhibited the binding of the radiolabeled salmon sperm dsDNA with the 90K recombinant protein (FIG. 19B). The same amount of crystalline bovine albumin, tested between 20 Tg to 2 mg in the incubations, did not cause the radiolabeled dsDNA to shift its position on the column, nor did the elution pattern of the albumin shift. This suggests the binding is specific.

Transfected dsDNA or dsRNA induces an increase in rat 90K tumor-associated immunostimulator protein coincident with increased MHC Class I gene expression. The expression correlates with Class I rather than Class II. It was previously shown that 90K tumor-associated immunostimulator could induce Class I expression when given to tumor cells. The 90K tumor-associated immunostimulator can bind ds nucleic acids. These data suggest that ds nucleic acid-induced 90K immunostimulator is not only a component of the immune response to ds nucleic acids, but also may be an intermediate in its action.

Aside from showing the 90K tumor-associated immunostimulator is a component of the ds nucleic acid immune induction response, these data raise an important link between ds nucleic acids and their role in tumor cells and AIDS. Studies of tumor cells have shown that dDNA is present in the cytoplasm (A. Solage & R. Laskove, Eur. J. Biochem. 60: 23-33 (1975); R. Hegger & H. Abken, Physiol. Chem. Phys. Med. NMR 27: 321-328 (1995)). Were dsDNA in the cytoplasm to increase 90K synthesis as well as enhance Class I levels, which is a reasonable likelihood, since Class I levels can increase on the surface of tumor cells, this would subject the tumor cell to immune regulation similar to a cell invaded by a bacteria or virus or subjected to tissue injury (J. Wekerle, Nature Medicine 4: 770-771 (1998); C. Benoist and D. Mathis, Nature 394: 227-228 (1998); G. Scambia, et al., Anticancer Res. 8, 761-764 (1998); S. Iacobelli, et al., Br. J. Cancer 69: 172-176 (1994); O. Fusco, et al., Int. J. Cancer 79: 23-26)). This data, thus, reinforces the possibility that ds nucleic acids play an important role in the immune response to oncogene-induced cell “injury”. The ds nucleic acids would induce a controlled immune response, similar to a viral infection, causing bystander activation of the immune system. This could induce tumor cell destruction by cytotoxic immune cells or antibody mediated destruction (H. Wekerle, Nature Medicine 4: 770-771 (1998); C. Benoist & D. Mathis, Nature 394: 227-228 (1998)). The ds nucleic acids become a means of therapeutic immuno-intervention to enhance tumor rejection by bystander activation of dormant autoreactive cells. This is consistant with action of 90K tumor-associated immunostimulator to increase NK and LAK cytotoxic effector cell generation (A. Ullrich, et. al., J. Biol. Chem. 269: 18401-18407 (1994)).

High levels of the 90K protein are also found in the serum of patients infected by the human immunodeficiency virus (HIV), even in the apparent absence of neoplastic complications (C. Natoli, et al., J. Infect. Dis. 164: 616-617 (1991); S. Iacobelli, et al., J. Infect Dis. 164: 819 (1991); C. Natoli, et al., J. AIDS 6: 370-375 (1993); N. Briggs, AIDS Res. Hum. Retroviruses 9: 81-816 (1993); S. Iacobelli, et al., J. AIDS 10: 450-456 (1995)). The levels of 90K in the serum have been linked to therapeutic efficacy (C. Natoli, et al., J. Infect. Dis. 164: 616-617 (1991); S. Iacobelli, et al., J. Infect. Dis. 164: 819 (1991); C. Natoli, et al., J. AIDS 6: 370-375 (1993); N. Briggs, AIDS Res. Hum. Retroviruses 9: 811-816 (1993); S. Iacobelli, et al., J. AIDS 10: 450-456 (1995)). The possibility thus exists that ds nucleic acids can become a means of therapeutic immuno-intervention in AIDS by bystander activation of dormant immune cells, thereby reawakening the immune cell suppressive state in these patients. The dsDNA-induced increase in Class I and the 90K immunostimulator could be evoked in almost any cell, not necessarily the tumor cell, since the effect of ds nucleic acids is ubiquitous in all cells tested (Example 1) and since the 90K tumor-associated immunostimulator is synthesized in normal cells throughout the body, as illustrated by its presence in thyrocytes.

We have shown that a viral promoter can increase 90K RNA levels and that ds nucleic acids increase 90K gene expression even more than KIFN. Viruses or viral promoters can increase Class I and Class II gene expression in cells (D. S. Singer & J. E. Maguire, CRC Crit. Rev. Immumol. 10: 235-257 (1990); J. P.-Y. Ting & A. S. Baldwin, Curr. Opin. Immunol. 5: 8-16 (1993)), as exemplified in the experiments described herein on MHC Class I RNA levels. Thus, a virus or its promoter coordinately should increase MHC gene and 90K expression in a cell. The increase in Class I and 90K is part of the host immune defense mechanism to protect the cell or organism. Normally, hormones such as TSH or insulin, which regulate 90K gene expression in the thyrocyte, would place that defense mechanism under cell control, both positive (increased gene expression) and negative (increased turnover or degradation). Thus, the 90K would normally regulate the host defense mechanism against viruses which might perturb the cell and might contribute to the control of regulated growth, preventing a tumorigenic state. In tumors, where normal hormone regulation is lost, synthesis of the 90K may be deregulated, degradation might be minimized, intact protein secreted, and a last ditch host defense mechanism to increase Class I levels and generate NK and LAK cytotoxic killer cells might be initiated. The ds nucleic acids can initiate this, as evidenced by their ability to increase MHC genes in cells treated with TSH as well as cells maintained without TSH (Example 3; FIG. 6) and by the ability of ds polynucleotides to increase gene expression of the 90K tumor-associated immunostimulator.

The present data concerning the role of 90K gene expression and its regulation by ds nucleic acids are novel and offer a potential therapeutic impact on the control of viruses, bacteria, or tissue injuries to cell, as well as tumors, either directly or by the development of drugs which can block their action.

The close correlation of 90K and Class I RNA increases, but not Class II increases, emphasizes the importance of abnormal Class I elevations as a trigger for autoimmune disease (M. Saji, et al., J. Clin. Endocrinol. Metab. 75: 871-878 (1992); L. D. Kohn, et al., Intern. Rev. Immunol. 9: 135-165 (1992); E. Mozes, et al., Science 261: 91-93 (1993); D. S. Singer, et al., J. Immunol. 153: 873-880 (1994); L. D. Kohn, et al., in Thyroid Immunity, D. Rayner and B. Champion (Eds.), R. G. Landes Biomedical Publishers, Texas, pp. 115-170 (1995)). The ds nucleic acids, resultant from virus, bacteria, oncogenic, or environmental “insults” to the tissue increase Class I predominantly. Class II is increased, but less so, because transcription factors important to regulate Class I, the cis elements with which they interact, and the coregulators which affect both, for example the Y box transcription factors, CIITA, and the CRE, are common factors or motifs in each. The resultant bystander activation of T cells leads to cytokine production, generation of KIFN, and an additive or synergistic response of the cell to the ds nucleic acid initial insult. This is a part of a host defense mechanism which aims to kill or thwart, repair or redress, the injury. Autoimmunity becomes the consequence of the immune cell protective mechanism initiated by the ds nucleic acid trigger. Any therapy must not thwart the protective mechanism but also must not allow excesses of the protective mechanism which express themselves as autoimmune disease. In this sense methimazole, its derivatives and tautomeric cyclic thiones, are ideal candidate drugs, since they have a minimal effect on the normal expression of the genes, but a profound effect on the ds nucleic acid or KIFN-induced elevations. The possibility, therefore, exists that drugs enhancing or inhibiting the ds nucleic acid action will be found that do not cause adverse effects on thyroid function as does methimazole or even the normal function of the cell.

### Example 7

DOUBLE STRAND POLYNUCLEOTIDE REGULATE CELL CYCLE PROGRESSION (GROWTH) DIFFERENTLY FROM K-INTERFERON: THE EFFECTS OF METHIMAZOLE AND 5-PHENYLMETHIMAZOLE ARE ALSO DIFFERENT ON CELL CYCLE PROGRESSION.

In previous examples, it was evident that transfection of double strand polynucleotides into cells could increase expression of a multiplicity of genes, not only MHC class I and class II. Some of these genes are clearly involved in the growth and function of the cell, for example the NF-PB, MAP Kinase, and JAK/Stat genes. Further, evidence exists in the FRTL-5 cell model that the expression of the thyrotropin receptor (TSHR) which controls the growth and function of the cell, is coregulated with the MHC genes and there are common transcription factors regulating the three genes (M. Saji, et al., Endocrinology 130: 520-523, (1992); M. Saji, et al., Proc. Natl. Acad. Sci. U.S.A. 89: 1944-1948 (1992); M. Saji, et al., J. Clin. Endocrinol. Metab. 75: 871-878 (1992); H. Shimura, et al., J. Biol. Chem. 268: 24125-24137 (1993); H. Shimura, et al., Mol. Endocrinol. 8: 1049-1069 (1994); Y. Shimura, et al., J. Biol. Chem. 269: 31908-31914 (1994); M. Bifulco, et al., J. Biol. Chem. 270: 15231-15236 (1995); C. Giuliani, et al., J. Biol. Chem. 270: 11453-11462 (1995); L.D. Kohn, et al., in Thyroid Immunity, D. Rayner & B. Champion (Eds), R. G. Landes Biomedical Publishers, Austin/Georgetown, Texas, pp. 115-170 (1995); L. D. Kohn, et al., Vitamins and Hormones 50: 287-384 (1995); H. Shimura, et al., Mol Endocrinol. 9: 527-539 (1995); M. Ohmori, et al., Mol. Endocrinol. 10: 1407-1424 (1996); M. Ohmori, et al., Mol. Endocrinol. 10: 76-89 (1996); D. S. Singer, et al., U.S. Pat. No. 5,556.754 (1996); A. Hirai, et al., J. Biol. Chem. 272:13-16 (1997); L. D. Kohn, Thyroid 7:493-498 (1997); M. Saji, et al., J. Biol. Chem. 272: 20096-20107 (1997); D. S. Singer, et al., Crit. Rev. Immunol. 17: 463-468 (1997); P. L. Balducci-Silano, et al., Endocrinology 139: 2300-2313 (1998); V. Montani, et al., Endocrinology 139: 280-289 (1998); V. Montani, et al., Endocrinology 139:290-302 (1998); Y. Noguchi, et al., J. Biol. Chem. 273:3649-3653 (1998); K. Suzuki, et al., Proc. Natl. Acad. Sci., U.S.A. 95: 8251-8256 (1998); S.-I. Taniguchi, et al., Mol. Endocrinol. 12: 19-33 (1998)). These data indicated that double strand polynucleotides would regulate genes important for growth and function of a cell, since coordinate control was necessary to regulate immune self defense mechanisms maintaining self tolerance.

Consistent with these conclusions, previous studies indicated that KIFN inhibited cell growth and function (M. Platzer, et al, Endocrinology 121: 2087-2092 (1987); T. Misaki, et al., Endocrinology 123: 2849-2855 (1998); M. Zakarija, et al., Mol. Cell. Endocrinol. 58: 329-336 (1988)). Similarly, several reports indicated methimazole inhibited cell growth (S.-I. Taniguchi, et al., Endocrinology 124: 2046-2051 (1989); P. Smerdely, et al., Endocrinology 133: 2403-2406 (1993)).

The present studies were therefore undertaken to see if ds nucleic acids, like KIFN, similarly regulated growth and the genes controlling growth processes. They were also undertaken to see whether compound 10 (5-phenylmethimazole) behaved like methimazole (MMI) as an inhibitor of cell cycle and growth and whether the MMI or compound 10 affected the double strand polynucleotide regulation of the genes linked to growth and function as well as those linked to MHC gene expression and increased expression of antigen presenting genes.

Experimental Protocol

Materials—Highly purified bovine TSH was obtained from the hormone distribution program of the National Institute of Diabetes and Digestive and Kidney Diseases, National Institutes of Health (NIDDK-bTSH 1-1; 30 U/mg), or was previously described preparation, 26±3 U/mg, homogeneous in the ultracentrifuge, about 27,500 in molecular weight, with the amino acid and carbohydrate composition of TSH (L. D. Kohn and R. J. Winand, J. Biol. Chem. 250: 6503-6508 (1975)). MMI and insulin were from the Sigma Chemical Co. (St. Louis, Mo.); rat recombinant KIFN was from GIBCO Laboratories Life Technologies, Inc. (Grand Island, N.Y.).

Cell Culture—FRTL-5 rat thyroid cells were a fresh subclone (F1) with the properties described (F. S. Ambesi-Impiombato, U.S. Pat. No. 4,608,341 (1986); L. D. Kohn, et al., U.S. Pat. No. 4,609,622 (1986); F. S. Ambesi-Impiombato and H. Perrild, FRTL-5 Today, Int Congress Series 818, Excerpta Medica, Amsterdam, The Netherlands, pp. 1-286 (1989); L. D. Kohn, et al., in Thyroid Immunity, D. Rayner and B. Champion (Eds.), R. G. Landes Biomedical Pub., Austin and Georgetown, Tex. pp. 115-170 (1995); L. D. Kohn, et al., Vitamins and Hormones 50: 287-384 (1995)). They were grown in Coon's modified F-12 medium containing 5% heat-treated, mycoplasma-free calf serum (GIBCO), 1 mM nonessential amino acids (GIBCO), and a mixture of six hormones (6H) containing bovine TSH (1×10⁻¹⁰M), insulin (10 Tg/ml), cortisol (0.4 ng/ml), transferrin (5 Tg/ml); glycyl-L-histidyl-L-Lysine acetate (10ng/ml), and somatostatin (10ng/ml). Cells were diploid and between their 5^{th} and 25^{th} passage. Fresh medium was added every 2 or 3 days and cells were passaged every 7-10 days. In some experiments, as noted, cells were grown to near confluency in 6H medium then maintained in 5H medium (which contains no TSH) or 4H medium (with no TSH and no insulin) for 6-8 days before experiments were initiated. Treatment with 5H or 4H medium synchronizes the cells by piling them up in G₀/G₁ (A. Hirai, et al., J. Biol. Chem. 272: 13-16 (1997); Y. Noguchi, et al., J. Biol. Chem. 273: 3649-3653 (1998)).

DNA Staining and Cell Cycle Analysis—The procedure used was a modification of that described (P. Smerdely, et al., Endocrinology 133: 2403-2406 (1993)). It used the Cycle TEST PLUS DNA Reagent Kit (Becton Dickinson, San Jose, Calif.); and FACS analysis was performed according to the manufacturer's instructions. Briefly, 5×10⁵ cells were incubated with 250 T1 of Solution A (trypsin buffer) for 10 min at room temperature, then 200 T1 of Solution B (trypsin inhibitor and ribonuclease A buffer) was added and further incubated for 10 min. Cold Solution C (propidium iodide stain solution) was added and incubated for 10 min at 4° C. in the dark. FACS analysis was performed using FACScan (Becton Dickinson, San Jose, Calif.). Each analysis was performed in triplicate on cells from 3 different plates; the histogram had at least 10,000 events and a coefficient of variation less than 5%.

Results

Double strand polynucleotides increase cell cycle progression (Table 2) whereas KIFN inhibits progression (M. Platzer et al, Endocrinology 121: 2087-2092 (1987); T. Misaki, et al., Endocrinology 123: 2849-2855 (1998); M. Zakarija, et al., Mol. Cell. Endocrinol, 58: 329-336 (1988)). Both methimazole and compound 10 inhibit the action of the ds nucleic acids.

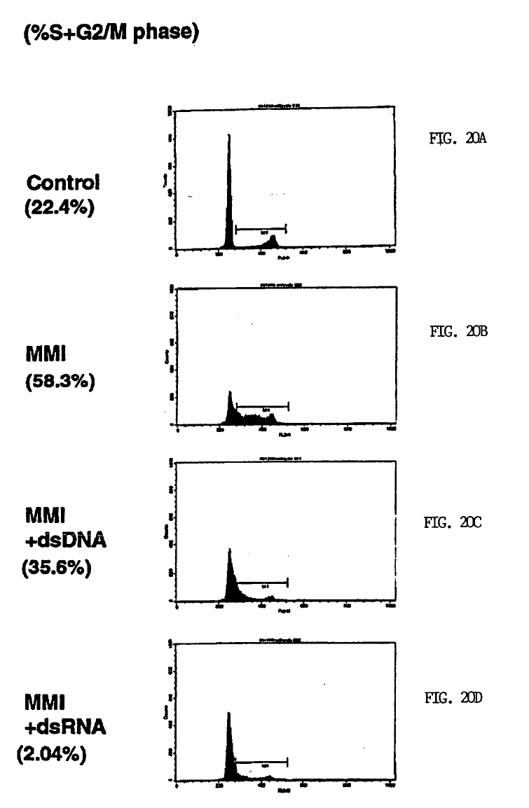

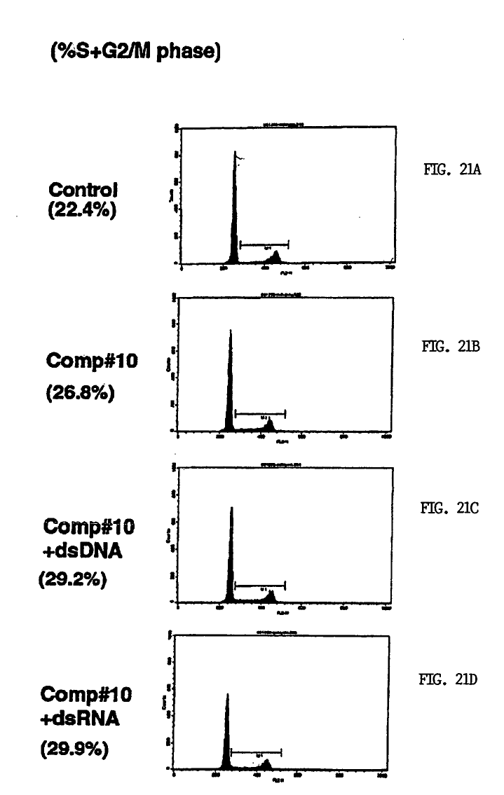

In the experiment above, there was a minimal direct methimazole effect on the cell cycle because the cells were maintained in 4H medium without insulin; methimazole action requires insulin (O. Isozaki, et al., Mol. Endocrinol, 3: 1681-1692 (1989); O. Isozaki, et al., Endocrinology 128: 3113-3121 (1991)). In a separate experiment (FIG. 20) using cells maintained in 5H medium (with insulin) for 6 days then stimulated with a physiological amount of TSH, 1×10⁻¹⁰M, we observed that methimazole caused cells to arrest in the G₂/M₁ phase. In this experiment, FRTL-5 cells were grown to near confluency in 6H medium, then shifted to 5H medium without TSH for 6 days. The experiments were initiated by returning the cells to 6H medium to reinitiate the cell cycle. Cells were treated with 5 mM methimazole and transfected or not with dsDNA or dsRNA. After 36 hours they were subjected to cell cycle analysis. Compound 10 had no such effect (FIG. 21). In this experiment, FRTL-5 cells were grown to near confluency in 6H medium, then shifted to 5H medium without TSH for 6 days. The experiments were initiated by returning the cells to 6H medium to reinitiate the cell cycle. Cells were treated with 0.5 mM 5-phenylmethimazole (compound 10) and transfected or not with dsDNA or dsRNA. After 36 hours they were subjected to cell cycle analysis. Double strand DNA reversed the methimazole effect (FIG. 20), consistent with its ability to increase growth; compound 10 had no effect on ds nucleic acid effects on cell cycle or the converse, under these conditions (FIG. 7).

These data reinforce the evidence that ds nucleic acids are different from KIFN in their mechanism of action and suggest that ds nucleic acids will alter the expression of genes other than MHC or other than those coding for antigen presenting molecules. The ds nucleic acids increase cell growth independent of TSH and independent of insulin. They therefore bypass normal hormonal regulatory control of thyroid growth. This phenomenon is characteristic of transformed cells and may reflect the fact tumor cells have been noted to have dsDNA in their cytoplasm (A. Solage & R. Laskov, Eur. J. Biochem. 60: 23-33 (1975); R. Hegger & H. Abken, Physiol. Chem. Phys. Med. NMR. 27: 321-328 (1995)). The complex nature of growth and cell cycle events suggests, therefore, that ds nucleic acid perturbation of the cell cycle may offer new information about genes important for growth and function. Examining these genes in chip arrays in cells treated with or not treated with ds nucleic acids may be a means to study these phenomena and may uncover new points of drug control to regulate the autoimmune host defense mechanism and the growth or function of cells which are closely coordinated events in the cell cycle.

The data additionally emphasize the fact that methimazole and tautomeric cyclic thiones have different effects on the cell, in this case different effects on growth, and in previously demonstrated work, different effects on finction (L. D. Kohn, et al., Methimazole derivatives and tautomeric cyclic thiones to treat autoimmune disease. U.S. Patent application submitted Aug. 31, 1998). Methimazole counteracts the effect of ds nucleic acids on growth; compound 10, a tautomeric cyclic thione does not. This may provide a more select drug which blocks an adverse or excess autoimmune response leading to disease expression but will not impare normal growth and function. This emphasizes that the present observations (Examples 1 through 7) define a new platform to develop drugs with selective effects on autoimmune defense, positive and negative.

TABLE 2
Effect of dsDNA or dsRNA on cell cycle progression measured
as the percentage of cells in S + G2/M phase.

| Treatment | Control | dsDNA | dsRNA |
|---|---|---|---|
| No Treatment | 7.7 | 20.4 | 17.8 |
| +Methimazole 5 mM | 6.3 | 11.4 | 7.4 |
| +5-Phenylmethimazole (Compound 10) | 10.5 | 11.0 | 4.5 |
| 0.5 mM | | | |

FRTL-5 cells were grown to near confluency in 6 H medium, then shifted to 4 H medium without insulin or TSH for 6 days, i.e. to a nongrowth state. Cells were transfected with dsDNA or dsRNA and subjected to cell cycle analysis.

### Example 8

DOUBLE STRAND POLYNUCLEOTIDE INDUCTION OF MHC GENE EXPRESSION AND EXPRESSION OF GENES IMPORTANT FOR ANTIGEN PRESENTATION CAN BE USED TO ASSES VIRAL REPLICATION

Since double strand nucleic acides introduced into the cytoplasm of host cells can induce increased expression of MHC genes, genes important for antigen presentation, and genes related to the growth and finction of the cell, meausrement of these molecule can be used to evaluate viral infection and replication within the cell.

The preferred current method to assess viral infection or replication depends on the demonstration of a known and expressed and/or secreted viral protein. However, this is not always applicable until an antibody against such a protein is raised and related assay systems are developed. PCR-based methods, which might also be used, are always controversial because of the possibility of false positives due to contamination and cross reactivity with host proteins, the fundamental point of molecular mimicry.

Measuring MHC and related molecule after viral infection provides a simple, but powerful tool which is applicable to measure any kind of viral replication within a host cell at an early stage of infection, i.e., when host genes are first subverted and host genes are turned on during the initial host defense response to this invasion by foreign DNA or RNA. Many approaches have been taken trying to transfect viral cDNA or RNA in cultured cells or animals in order to test viral vaccines or to simply try to establish an in vitro system of persistent infectious cells for further studies of the viral replicative mechanisms. However, one of the difficulties is the lack of an assay system to measure viral replication.

One typical example is a single strand RNA virus, such as hepatitis C virus. To date, there is no in vitro culture system for hepatitis virus. This is the major factor delaying the production of effective vaccines and other effective therapeutic approaches.

Results

We have shown (Examples 1 through 3) that only double strand RNA, not single strand RNA, can induce MHC class I, TAP transporter, and proteosome protein, LMP2 in the human hepatoblastoma cell line, HuH7 (Examples 1 and 2). In experiments where full length, single strand hepatitis virus RNA was transfected into the HuH7 liver cell line, exactly as described for herpes simplex virus in Example 1, we observed increased expression of MHC class I, TAP transporter, and the proteasome, LMP2, as detailed in Examples 1 through 3. The same experiment using rat FRTL-5 cells did not result in increases in these genes; however, hepatitis C virus is known to be a liver- and human-cell-specific virus.

This evidence indicates that the single strand RNA transfected into the cell was able to replicate to form double strand forms, since only double strand RNA can increase expression of these genes in these cells. This indicates that the viral RNA injected into the host cell was able to capture host genes needed for its replication and induce the increased expression of host genes important to defend the host cell from viral injury signaled by the presenceof the foreign double strand nucleic acid in the cytoplasm. The host cell responded, therefore, to the double strand RNA formed during the replication process.

These results are consistent with our hypothesis that genes important for the growth and function of the cell are coregulated with the MHC genes and there are common transcription factors regulating the three genes (M. Saji, et al., Edocrinology 130: 520-523, (1992); M. Saji, et al. Proc. Natl. Acad. Sci. U.S.A. 89: 1944-1948 (1992); M. Saji, et al., J. Clin. Endocrinol. Metab. 75: 871-878 (1992; H. Shimura, et al., J. Biol. Chem. 268: 24125-24137 (1993); H. Shimura, et al., Mol. Endocrinol. 8: 1049-1069 (1994); Y. Shimura, et al., J. Biol. Chem. 269: 31908-31914 (1994); M. Bifulco, et al., J. Biol. Chem. 270: 15231-15236 (1995); C. Giuliani, et al., J. Biol. Chem. 170: 11453-11462 (1995); L.D. Kohn, et al., in Thyroid Immunity, D. Rayner & B. Champion (eds), R. G. Landes biomedical publishers, Austin/Georgetown, Tex., pp. 115-170 (1995); L. D. Kohn, et al., Vitamins and Hormones 50: 287-384 (1995); H. Shimura, et al., Mol. Endocrinol. 9: 527-539 91995); M. Ohmori, et al., Mol. Endocrinol. 10: 1407-1424 (1996); M. Ohmori, et al., Mol Endocrinol. 10: 76-89 (1996); D. S. Singer, et al., U.S. Pat. No. 5,556.754 (1996); A. Hirai, et al., J. Biol. Chem. 272: 13-16 (1997); L. D. Kohn, Thyroid 7: 493-498 (1997); M. Saji, et al., J. Biol. Chem. 272: 20096-20107 (1997); D. S. Singer, et al., Crit. Rev. Immunol. 17: 463-468 (1997); P. L. Balducci-Silano, et al, Endocrinology 139: 2300-2313 (1998); V. Montani, et al., Endocrinology 139: 280-289 (1998); V. Montani, et al., Endocrinology 193:290-302 (1998); Y. Noguchi, et al., J. Biol. Chem. 273:3649-3653 (1998); K. Suzuki, et al., Proc. Natl. Acad. Sci., U.S.A, 95: 8251-8256 (1998); S.-I. Taniguchi, et al., Mol. Endocrinol. 12: 19-33 (1998)). Thus, two of the transcription factors identified as common factors in MHC gene expression and expression of genes important for growth and cell function are single strand binding proteins which bind single strand RNA as well as DNA, single strand binding protein-1 and the Y box protein (M. Ohmori, et al., Mol. Endocrinol. 10: 1407-1424 (1996); M. Ohmori, et al., Mol. Endocrinol. 10: 76-89 (1996); L. D. Kohn, et al., in Thyroid Immunity, D. Rayner & B. Champion (eds), R. G. Landes Biomedical Publishers, Austin/Georgetown, Tex., pp. 115-170 (1995); L. D. Kohn, et al., Vitamins and Hormones 50: 287-384 (1995)). Both proteins are important for replication of single strand RNA viruses (M. Ohmori, et al., Mol. Endocrinol. 10: 1407-1424 (1996); M. Ohmori, et al., Mol. Endocrinol. 10: 76-89 (1996)).

These data are consistent, therefore, with the conclusion that double strand polynucleotides would regulate genes important for growth and function of a cell, since coordinate control was necessary to regulate immune self defense mechanisms maintaining self tolerance. These observations indicate that transfection of single strand, full length, viral RNA or DNA and assessing induction of MHC genes and/or genes related to antigen presentation, together with known dsDNA and dsRNA as a control, will provide a novel and general method to evaluate active replication of viral nucleic acids from such constructs. It will be a procedure able to measure infection and replication of virus itself, even in a case of an unknown virus.

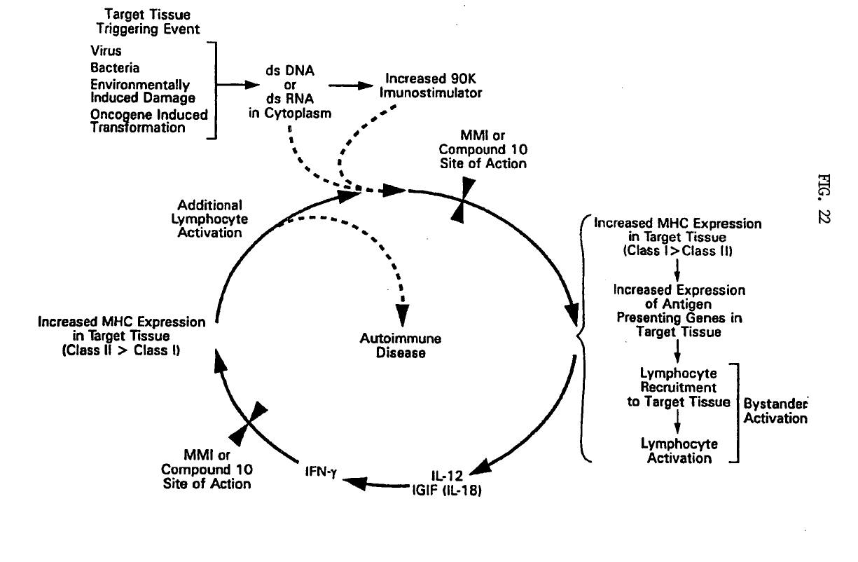

In U.S. Patent application submitted Aug. 31, 1998 (L. D. Kohn, et al., Methimazole derivatives and tautomeric cyclic thiones to treat autoimmune disease) we showed that the primary effect of methimazole, methimazole derivatives, and tautomeric cyclic thiones was to prevent or reverse the action of interferon to increase MHC gene expression and exacerbate an immune response initiated by an unknown initial or primary insult on the target tissue which initiates the immune response (FIG. 22). In the present invention we identify a probable causative mechanism whereby viruses, bacteria, environmental injuries, or oncogene transformation, for example, introduce double strand polynucleotides into the cytoplasm of target tissue cells and increase MHC gene expression, increase the expression of genes important for antigen presentation to immune cells, activate gene products important for antigen presentation to immune cells, and increase expression of or activate products of genes which control host cell function and growth which are coordinately regulated in the host defense system (FIG. 22). We show that methimazole and a tautomeric cyclic thione (5-phenylmethimazole), in particular can inhibit this processing addition to their action on the interferon induced arm of the autoimmune defense mechanism (FIG. 22).

Tautomeric cyclic thiones, in particular 1,3-dimethyl-4-phenylimidazoline-2-thione is said to exhibit antivrial properties against herpes simplex and vaccinia viruses. Together with the data in Examples 1 through 7, Example 8 raises the probability that the compounds which are identified by assays to inhibit or prevent the action of the double strand polynucleotides by viruses, viral DNA, or viral RNA will be, at least in some cases, antiviral agents and the converse, some antiviral or other agents will be antiiummune, as is the case for metronidazole (L. D. Kohn, et al., Methimazole derivatives and tautomeric cyclic thines to treat autoimmune disease. U.S. Patent aplication submitted Aug. 31, 1998). Moreover, the test system described in this example should provide a simple screening process for discovering such drugs.

1. A method of increasing immune recognition of a mammalian cell in a subject comprising:
(a) obtaining a nonimmune cell from a subject in need of such treatment;
(b) introducing a sequence nonspecific double-stranded polynucleotide greater than 25 nucleotides in length into the cell and thereby activating expression of a gene or gene product, wherein such activation is involved in antigen presentation and which increases the ability of the cell to present antigen to an immune cell; and
(c) re-introducing the cell into the subject.

2. A method of increasing immune recognition of a nonimmune cell in a subject comprising:
(a) obtaining a cell from a subject in need of such treatment;
(b) introducing a sequence nonspecific double-stranded polynucleotide greater than 25 nucleotides in length into the cell, thereby activating expression of a gene or gene product that increases immune recognition;
(c) introducing an antigen into the cell; and
(d) re-introducing the cell into the subject.

3. A method of inducing an autoimmune response in a subject comprising:
(a) identifying a subject in need of such treatment; and
(b) introducing a sequence nonspecific double-stranded polynucleotide greater than 25 nucleotides in length into a nonimmune cell within the subject and thereby activating expression of a gene or gene product, wherein such activation is involved in antigen presentation and which induces an autoimmune response within the subject.

4. A method of increasing immune recognition of a mammalian immune cell in a subject comprising:
(a) obtaining an immune cell from a subject;
(b) introducing a sequence nonspecific double-stranded polynucleotide greater than 25 nucleotides in length into the immune cell and thereby activating expression of a gene or gene product that increases immune recognition, wherein the polynucleotide does not contain a stimulatory CpG motif and wherein such activation is involved in antigen presentation; and
(c) re-introducing the immune cell into the subject.

5. A method of increasing immune recognition of a mammalian immune cell in a subject comprising:
(a) obtaining an immune cell from a subject;
(b) introducing a sequence nonspecific double-stranded polynucleotide greater than 25 nucleotides in length into the immune cell and thereby activating expression of a gene or gene product that increases immune recognition, wherein the polynucleotide is a noncoding polynucleotide sequence and wherein such activation is involved in antigen presentation; and
(c) re-introducing the immune cell into the subject.

6. The method of claim 1, 2, 4, or 5 wherein the gene or gene product associated with increased immune activation is selected from the group consisting of MHC class I, MHC class II, TAP-1, TAP-2, a proteosome subunit, HLA-DM, invariant chain, RFXA, B7 co-stimulatory molecule, PKR, beta-interferon, MAP kinase, NF-kB, JAK, and STAT genes and gene products.

7. A method of increasing immune recognition of a monocyte or dendritic cell within a subject comprising:
(a) obtaining a monocyte or dendritic cell from a subject;
(b) introducing a sequence nonspecific double-stranded polynucleotide greater than 25 nucleotides in length into the monocyte or dendritic cell, wherein the polynucleotide does not contain a CpG motif, and thereby activating expression of a gene, or gene product or gene and gene product that increases immune recognition, wherein such activation is involved in antigen presentation and which increases the ability of the monocyte or dendritic cell to present antigen to an immune cell of the subject; and
(c) re-introducing the cell into the subject.

8. A method of increasing immune recognition of a mammalian immune cell in a subject comprising:
(a) obtaining an immune cell from a subject;
(b) introducing a sequence nonspecific double-stranded polynucleotide greater than 25 nucleotides in length into the immune cell, thereby activating expression of a gene or gene product that increases immune recognition, wherein such activation is involved in antigen presentation and wherein the polynucleotide contains one or more CpG motifs, which if methylated do not decrease activity of the polynucleotide; and
(c) re-introducing the immune cell into the subject.

9. The method of claim 1, 2, 3, 4, 5, 7, or 8 wherein the double-stranded polynucleotide is introduced by the method selected from the group consisting of transfection, microinjection, viral infection of the cell, and cell injury.

10. (The method of claim 3 wherein the double-stranded polynucleotide is introduced by microinjection.

11. A method of increasing immune recognition of a nonimmune mammalian cell in a subject comprising: (a) identifying a subject having a nonimmune mammalian cell in need of such treatment; (b) introducing a sequence nonspecific double-stranded polynucleotide greater than 25 nucleotides in length into the cell within the subject and thereby activating expression of a gene or gene product, which increases the ability of a cell to present antigen to an immune cell.

12. The method of claim 1, 4, 5, 6, 7, 8, 11, 46, 60, 68, 72, or 74, wherein the cell expresses an autoantigen.

13. The method of claim 1, 2, 3, or 11 wherein the cell is selected from the group consisting of somatic cell, antigen presenting cell and thyroid cell.

14. The method of claim 13 wherein the cell is a thyroid cell.

15. The method of claim 7, 8, or 11, wherein the gene or gene product that increases immune recognition is selected from the group consisting of MHC class I and class II genes and gene products, peptide processing genes and gene products, class II regulatory genes and gene products, co-stimulatory molecule gene and gene products and beta-interferon.

16. The method of claim 6 or 15 wherein the gene or gene product is derived from the major histocompatibility complex (MHC) and wherein a MHC Class I expression increases greater than a MHC Class II expression as a function of time after introduction of concentration of the double-stranded polynucleotide.

17. The method of claim 6 or 15 wherein expression of the MHC molecule is accompanied by increased expression of an about 90 kilodalton tumor-associated immunostimulator.

18. The method of claim 4, 5, 7, or 8 wherein the method further comprises the step of introducing tumor cell RNA into the cell ex vivo.

19. The method of claim 1, 2, 3 or 7 additionally comprising forming an activated antigen presenting cell (APC).

20. The method of claim 1, 2, 4, 5, 7, 11, 46, 68, 72, or 74, wherein the polynucleotide introduced into the cells is single stranded RNA molecule that, when introduced into the cell, replicates to form a double stranded polynucleotide within the cell.

21. The method of claim 1, 2, 4, 5, 7, 8, 11, 68, 72, or 74, wherein the cell can induce an autoimmune response when injected into the subject.

22. The method of claim 1, 2, 4, 5, 7, 8, 11, 68, 72, or 74, wherein the cell recruits and activates T cells when injected into the subject.

23. The method of claim 1, 2, 4, 5, 7, 8, 11, 68, 72, or 74, wherein the cell produces at least one soluble mediator of immunity.

24. The method of claim 6 or 15 wherein increasing expression of the MHC molecule by double-stranded polynucleotide is additive to and independent of a gamma-interferon-mediated increase in immune recognition.

25. The method of claim 1, 2, 4, 5, 7, 8, 11, 68, 72, or 74, wherein the double-stranded polynucleotide is RNA that is introduced into the cell and wherein the polynucleotide initiates an antigen presenting response and does not initiate such antigen presenting response by acting through a cell surface receptor.

26. The method of claim 7 wherein the double-stranded polynucleotide does not contain any stimulatory CpG motifs.

27. The method of claim 1, 2, 7, or 11, wherein methylation of any CpG motifs within the polynucleotide does not effect the activity of the polynucleotide.

28. The method of claim 1, 2, 4, 5, 7, 8, 11, wherein the polynucleotide is a noncoding sequence.

29. The method of claim 1, 2, 4, 5, 7, 8, 46, 68, 72, or 74, wherein the method comprises the further step of treating the cells to prevent cell division prior to introducing the polynucleotide containing cell into a host organism.

30. The method of claim 1, 2, 4, 5, 7, 8, 11, 46, 68, 72, or 74, wherein neither strand of the polynucleotide encodes a molecule involved in antigen presentation.

31. The method of claim 1, 2, 4, 5, 7, 8, 11, 46, 68, 72, or 74, wherein the immune system of the subject recognizes one or more antigens presented by the cell.

32. The method of claim 67 wherein expression of both MHC Class I and Class II molecules in or on the cell are increased.

33. The method of claim 1, 2, 4, 5, 7, 8, 11, 46, 68, 72, or 74, wherein the double-stranded polynucleotide comes from the mammalian cell's nucleus or mitochondria.

34. The method according to claim 19 and further comprising introducing an antigen into the mammalian cell prior to introduction of the activated APC into the subject.

35. The method of claim 34 wherein introduction causes an autoimmune reaction in the host animal.

36. A screening method for a drug to regulate antigen presentation comprising:
(a) introducing a double-stranded polynucleotide into a mammalian cell; (b) measuring expression in or on the mammalian cell of at least one molecule selected from the group consisting of major histocompatibility complex (MHC) molecule and non-MHC molecule involved in antigen presentation; (c) mixing the mammalian cell with or without a candidate drug; and (d) measuring an increase or decrease in the mammalian cell's ability to present antigen after introduction of the double-stranded polynucleotide when incubations with or without the candidate drug are compared.

37. The method of claim 36 wherein the introduction of a double-stranded polynucleotide is coincident with or after the incubation with or without a candidate drug.

38. A method for drug screening comprising: (a) introducing double-stranded polynucleotide into a mammalian cell, (b) treating the cell with the drug before, coincident with or after introducing double-stranded polynucleotide, and (c) measuring expression of major histocompatibility complex (MHC) molecules and about a 90 kilodalton tumor-associated imunostimulator gene expression about 12 or more hours after treating the cell with the drug in step (b) is performed.

39. The method of claim 38 wherein the drug is MMI, an MMI derivative, a thione or a thione derivative.

40. A pharmaceutical composition wherein the composition includes a drug capable of preventing tissue damage caused by an autoimmune reaction, preventing atherosclerotic plague development, treating autoimmune disease, treating an infection, treating transplantation rejection, or treating tumor cells, comprising an effective amounts of methimazole, methimazole derivatives, or tautomeric cyclic thiones.

41. A DNA molecule comprising at least one of SEQ ID NOS: 1-16.

42. The method of claim 2, 4, 5, 7, 8, 11, 46, 68, 72, or 74, wherein the cell recruits and activates other T or B cells to enhance the immune response.

43. The method of claim 32 wherein increasing expression of the MHC molecule by double-stranded polynucleotide is additive to or independent of an interferon-mediated increase in expression of the MHC molecule.

44. The method of claim 25 wherein the double-stranded polynucleotide is RNA that increases β-interferon production by the immune or antigen presenting cell.

45. The method of claim 1, 4, 5, 8, 13, 46, 68, or 72, wherein the cell is a tumor cell and the subject has an increased ability to recognize and kill the tumor cell after such treatment.

46. A method of presenting antigen to the immune system of a mammal in need of immunotherapy comprising; (a) introducing double-stranded polynucleotide into a somatic mammalian cell ex vivo, which improves the ability of the mammalian cell to present antigen; (b) thereby increasing expression or activity of a molecule selected from the group consisting of MHC molecules, TAP-1, TAP-2, a proteosome subunit, HLA-DM, invariant chain, RFXA, B7 co-stimulatory molecule, PKR, MAP kinase, NF-KB, JAK, beta-interferon, and STAT; and (c. introducing the somatic cells into the mammal; wherein the cells induce an immune response by the mammal to an antigen.

47. A method of identifying differential expression of a sequence expressed in response to a double-stranded polynucleotide comprising:
(a) introducing the double-stranded polynucleotide into a mammalian cell;
(b) isolating expressed RNA sequences from the cell treated with the double-stranded polynucleotide and from a cell not treated with the double-stranded polynucleotide; and
(c) comparing the isolated RNA sequences of the treated cell with the untreated cell and identifying the sequences differentially expressed in the treated cell as compared to the untreated cell.

48. The method of claim 47 wherein the sequence is expressed at a higher level in the double-stranded nucleotide-treated cell than in the untreated cell.

49. The method of claim 47 wherein the sequence is expressed at a lower level in the double-stranded nucleotide-treated cell than in the treated cell.

50. The method of claim 47 wherein the mammalian cell is selected from the group consisting of non-immune cell, immune cell, antigen presenting cell, and thyroid cell.

51. The method of claim 47 wherein the double-stranded polynucleotide is introduced by a method selected from the group comprising transfection, microinjection, direct injection, viral infection, phagocytosis, oncogene transformation or cytoplasmic leakage.

52. The method of claim 47 wherein control cells or cells treated with double-stranded polynucleotide are also treated with a drug to prevent changes induced by the double-stranded polynucleotide.

53. The method of claim 52 wherein the drug is selected from the group consisting of MMI or an MMI derivative.

54. The method of claim 53 wherein the drug is a tautomeric cyclic thione.

55. A method of screening for a compound that regulates the effect of double-stranded polynucleotides, comprising:
(a) introducing the double-stranded polynucleotide into a mammalian cell;
(b) exposing or not exposing the cell to the compound before or with or after introducing the double-stranded polynucleotide;
(c) isolating the RNA of the cell (d) quantitatively comparing the relative level of expression of double-stranded polynucleotide responsive genes expressed in the cell in the presence of absence of the compound; and
(e) identifying and selecting compounds shown to regulate the effect of double-stranded polynucleotides.

56. The method of claim 55 wherein the double-stranded polynucleotide responsive genes are selected from the group comprising MHC genes, non-MHC genes, and growth-related genes.

57. A method of screening for a compound that regulates the effect of double-stranded polynucleotides, comprising
(a) transfecting a non-professional immune cell with an antigen before or after introducing into the cell a double-stranded polynucleotide;
(b) immunizing an animal with the cell to induce an autoimmune disease;
(c) treating the animal with a compound; and
(d) determining whether the compound regulates the effect of the double-stranded polynucleotide.

58. The method of claim 57 wherein the method of determining whether the compound regulates the effect of the double-stranded polynucleotide comprises:
(a) exposing or not exposing an animal to the compound;
(b) isolating the RNA of the cell from the relevant tissues;
(c) quantitatively comparing the relative level of expression of double-stranded polynucleotide responsive genes expressed in cell in the presence or absence of the compound; and
(d) identifying and selecting compounds shown to regulate the effect of double-stranded polynucleotides.

59. The method of claim 57 wherein the method of determining whether the compound regulates the effect of the double-stranded polynucleotide comprises:
(a) exposing or not exposing the animal to the compound; and
(b) identifying and selecting compounds shown to prevent or alleviate the symptoms of the disease.

60. A method for treating a mammalian disease that is sensitive to immunotherapy which comprises:
a) removing diseased cells from a mammal identified as having a disease that is sensitive to immunotherapy;
b) introducing a sequence nonspecific double-stranded polynucleotide greater than 25 nucleotides in length into the cells;
c) treating the cells to prevent cell division but permit other metabolic activity; and
d) re-introducing the treated cells into the mammal; wherein the cells induce an immune response by the mammal to a self antigen.

61. A method for the treatment of diseases, disorders, conditions or symptoms associated with an autoimmune disease in a subject comprising administering to the subject a pharmaceutical composition comprising a methimazole derivative or tautomeric cyclic thione or mixtures thereof in an amount effective for prevention, inhibition or suppression of diseases, disorders, conditions or symptoms associated with an autoimmune disease.

62. A method for the treatment of diseases, disorders, conditions or symptoms associated with inflammation in a subject comprising administering to the subject a pharmaceutical composition comprising a methimazole derivative or tautomeric cyclic thione or mixtures thereof in an amount effective for prevention, inhibition or suppression of diseases, disorders, conditions or symptoms associated with an autoimmune disease.

63. The method of claim 61, wherein the methods are used prophylactically to treat a subject at risk of developing an autoimmune disease.

64. The method of claim 62, wherein the pharmaceutical composition is selected from the group consisting of methimazole, metronidazole, 2-mercaptoimidazole, 2-mercaptobenzimidazole, 2-mercapto-5-nitrobenzimidazole, 2-mercapto-5-methylbenzimidazole, s-methylmethimazole, n-methylmethimazole, 5-methylmethimazole, 5-phenylmethimazole, and 1-methyl-2-thiomethyl-5(4)nitroimidazole.

65. The method of claim 61 wherein a drug is added to prevent gene expression induced by a virus during the preparation procedure.

66. A method of claim 65, wherein the pharmaceutical composition is 5-phenylmethimazole.

67. The method of claim 66 wherein the treatment is in addition to treatment with CpG motifs.

68. A vaccine for treating cancer comprising:
(a) a somatic mammalian cell with the enhanced ability to present antigen to the immune system wherein a sequence non-specific doubled-stranded polynucleotide greater than 25 nucleotides in length is introduced into the somatic mammalian cell ex vivo and which is capable of inducing an autoimmune response; and
(b) a pharmaceutically acceptable carrier.

69. A vaccine for treating cancer that is sensitive to immunotherapy which comprises;
a) an adjuvant comprising a sequence non-specific doubled-stranded polynucleotide greater than 25 nucleotides in length;
b) an antigen of interest; and
c) a pharmaceutically acceptable carrier.

70. A method for augmenting a vaccine response comprising administering an antigen and an adjuvant to a mammal in need of such treatment, wherein the adjuvant comprises a sequence non-specific doubled-stranded polynucleotide greater than 25 nucleotides in length.

71. The method of claim 70 wherein the treatment is in addition to treatment with CpG motifs used to enhance immune cell responsiveness.

72. A method for treating cancer that is sensitive to immunotherapy which comprises:
a) obtaining a somatic cell from a subject in need of treatment;
b) introducing a sequence non-specific doubled-stranded polynucleotide greater than 25 nucleotides in length into the somatic mammalian cell ex vivo, which causes the cell to have an increased ability to present antigen;
c) increasing the expression of one or more molecules involved in antigen presentation selected from the group consisting of MHC molecules, TAP-1, TAP-2, a proteosome subunit, HLA-DM, invariant chain, RFX5, B7 co-stimulatory molecule, PKR, MAP Kinase, NF-κ B, JAK, beta-interferon, and a STAT;
d) preparing the mammalian cell to make suitable for immunization; and
e) introducing the cell into a subject in need of such treatment.

73. The method of claim 72 wherein the polynucleotide is single stranded RNA molecule that, when introduced into the cell, replicates to form a double stranded polynucleotide within the cell.

74. A method for treating a patient with a cancer which is sensitive to immunotherapy comprising:
a) removing monocytes from the patient;
b) introducing a sequence non-specific doubled-stranded polynucleotide greater than 25 nucleotides in length into the monocytes ex vivo, which causes the monocytes to have an increased ability to present antigen;
c). introducing a tumor cell antigen into the monocytes wherein the antigen is selected from the group consisting of a protein, a peptide, an mRNA encoding antigen and a DNA encoding antigen; and
d). re-introducing the monocytes into the patient.

75. The method of claim 68 wherein methylation of any CpG motifs within the polynucleotide does not effect the activity of the polynucleotide.

76. The vaccine of claim 68, wherein the cell is a tumor cell.

77. The vaccine of claim 68, wherein the cell is a fibroblast and wherein tumor cell RNA is introduced into the cell ex vivo.

78. The vaccine of claim 68, wherein the vaccine is adapted for injection in muscle tissue of a mammal.


SEQUENCE LISTING (23 sequences)

SEQ ID NO: 1 - DNA, 21 bp, Rattus sp.
taccgtgaggacttgttagcg

SEQ ID NO: 2 - DNA, 20 bp, Rattus sp.
atgactcgatggtccacacc

SEQ ID NO: 3 - DNA, 20 bp, Rattus sp.
ggaacagtcgcttagatgcc

SEQ ID NO: 4 - DNA, 20 bp, Rattus sp.
cactaatggactcgcacacg

SEQ ID NO: 5 - DNA, 19 bp, Rattus sp.
aattgcaaccgtggagtcc

SEQ ID NO: 6 - DNA, 20 bp, Rattus sp.
aacacacaccagcagtagcc

SEQ ID NO: 7 - DNA, 21 bp, Rattus sp.
atcctcaacaaggaagaaggc

SEQ ID NO: 8 - DNA, 20 bp, Rattus sp.
gttcttcatccacaccacgg

SEQ ID NO: 9 - DNA, 21 bp, Rattus sp.
ccatacaccgaatctactggc

SEQ ID NO: 10 - DNA, 20 bp, Rattus sp.
ttgactgcatcagatcctgc

SEQ ID NO: 11 - DNA, 21 bp, Homo sapiens
aagctgtatctctaccttcag

SEQ ID NO: 12 - DNA, 21 bp, Homo sapiens
tttcaggatccgctctgccca

SEQ ID NO: 13 - DNA, 21 bp, Rattus sp.
acaaggtggatagtcacacgg

SEQ ID NO: 14 - DNA, 20 bp, Rattus sp.
ccagatgctgactgagaagc

SEQ ID NO: 15 - DNA, 21 bp, Rattus sp.
aagatcattctcactgcagcc

SEQ ID NO: 16 - DNA, 20 bp, Rattus sp.
tgaagacttctgctcggacc

SEQ ID NO: 17 - DNA, 20 bp, Rattus sp.
agcaagccagtcacagaagg

SEQ ID NO: 18 - DNA, 20 bp, Rattus sp.
gattcgacttggaagatgcc

SEQ ID NO: 19 - DNA, 2016 bp, Rattus sp.
gaattctacgccaggcaatggctcttctgtggctcctctctgtgttcttgctggttccagggactcaaggtgcaaaggatggagacatgcgcctggttaatggggcctcacccagtgagggccgcgtggagatcttctacagaggccggtgggggacactgtgcgacaacctctggaaccttttggatgcccacgtcttctgccgggccctgggctatgataatgctactccagcactgaacagagtcgccttcgggccaggaaagggaccaatcatgctggatgaggtggaatgcacagggaacgagtcgtcactggccaattgcagctccctgggctggatggtgagccactgcgggcatgagaaggacgcgggcgtggtctgctccaacgattccaggggcattcacatcctagacctctctggagagcttccagattcactgggccagatctttgacagccagcaggactgcgacctgttcatccaggtgacagggcagggacatggggacctgagcctctgtgcccacacactgatcctgcgcaccaaccccgaggcccaggccctgtggcaagtggtgggcagcagtgtcatcatgagagtggacgctgagtgcatgcctgtcgtcagagacttcctcaggtacttttactcccgaagaatcgaggtcagcatgtcttctgtcaagtgtctgcacaagctggcctccgcctatggagccacagagctccagggttactgtggacggctttttgtcaccctcctcccccaggaccccactttccatacgcccctggaactttacgagtatgcgcaggccaccggggactctgtgctggaagatctgtgtgtgcaattcctggcctggaacttcgagcctctgacacaggccgagtcctggttgtctgttcccaatgccttgatccaagctctcctccccaagagcgagctggctgtgtctagtgaactggatttgctgaaggcagtggaccagtggagcacagcgaccggcgcctcccacggggatgtagagcgcctggtggaacagatccgctttcctatgatgctgccccaggagctgtttgagctacagttcaacctgtccttgtaccaaggtcaccaggccctgttccagaggaagaccatggaggccctggagttccacacagtgcctctcaaagtgctggccaagtacagaagcctgaacctcaccgaggatgtctacaaaccccggctttacacctcttctacctggagtagcctgctgatggccggtgcctggagtacacaaagctacaaatacagacagttctacacatacaactatggctcacaatcccgctacagcagctaccagaacttccagaccccacaacaccccagcttcctcttcaaggacaagctgatctcctggtcagccacctacctccccaccatccagagttgctggaactatggcttctcgtgtacctctgacgagctccctgtactgggcctcaccacatccagttactccgatccaactatcggctacgagaacaaagcgctgatcctctgtggaggctacagtgtggtagatgtcaccacttttataggctctaaggcccctattccaggtacccaggagaccaatagttccaagaccccctccctctttccctgtgcctcaggggccttcagcagcttccgcgtggtcatccgccccttctacctcaccaactccactgacacggagtagatggtacatctcagcggtggggactcagacattcctgtgttccctccttggcctccagcctctctgtaggaacctccagcagcctgccaccagatttcccttagcttccactgtctccatgagctttaaatgtatctagaaggtttcagccagtactcactcctagatctgagagtctcaggcccccaattgtaggcagcaaggaggtcctgtgggattccccatcagtcacagtcactaatctgaaatcattaaagtggcacgtgcttctccg

SEQ ID NO: 20 - protein, 574 aa, Rattus sp.
MALLWLLSVFLLVPGTQGAKDGDMRLVNGASPSEGRVEIFYRGRWGTLCDNLWNLLDAHVFCRALGYDNATPALNRVAFGPGKGPIMLDEVECTGNESSLANCSSLGWMVSHCGHEKDAGVVCSNDSRGIHILDLSGELPDSLGQIFDSQQDCDLFIQVTGQGHGDLSLCAHTLILRTNPEAQALWQVVGSSVIMRVDAECMPVVRDFLRYFYSRRIEVSMSSVKCLHKLASAYGATELQGYCGRLFVTLLPQDPTFHTPLELYEYAQATGDSVLEDLCVQFLAWNFEPLTQAESWLSVPNALIQALLPKSELAVSSELDLLKAVDQWSTATGASHGDVERLVEQIRFPMMLPQELFELQFNLSLYQGHQALFQRKTMEALEFHTVPLKVLAKYRSLNLTEDVYKPRLYTSSTWSSLLMAGAWSTQSYKYRQFYTYNYGSQSRYSSYQNFQTPQHPSFLFKDKLISWSATYLPTIQSCWNYGFSCTSDELPVLGLTTSSYSDPTIGYENKALILCGGYSVVDVTTFIGSKAPIPGTQETNSSKTPSLFPCASGAFSSFRVVIRPFYLTNSTDTE

SEQ ID NO: 21 - protein, 585 aa, Homo sapiens
MTPPRLFWVWLLVAGTQGVNDGDMRLADGGATNQGRVEIFYRGQWGTVCDNLWDLTDASVVCRALGFENATQALGRAAFGQGSGPIMLDEVQCTGTEASLADCKSLGWLKSNCRHERDAGVVCTNETRSTHTLDLSRELSEALGQIFDSQRGCDLSISVNVQGEDALGFCGHTVILTANLEAQALWKEPGSNVTMSVDAECVPMVRDLLRYFYSRRIDITLSSVKCFHKLASAYGARQLQGYCASLFAILLPQDPSFQMPLDLYAYAVATGDALLEKLCLQFLAWNFEALTQAEAWPSVPTDLLQLLLPRSDLAVPSELALLKAVDTWSWGERASHEEVEGLVEKIRFPMMLPEELFELQFNLSLYWSHEALFQKKTLQALEFHTVPFQLLARYKGLNLTEDTYKPRIYTSPTWSAFVTDSSWSARKSQLVYQSRRGPLVKYSSDYFQAPSDYRYYPYQSFQTPQHPSFLFQDKRVSWSLVYLPTIQSCWNYGFSCSSDELPVLGLTKSGGSDRTIAYENKALMLCEGLFVADVTDFEGWKAAIPSALDTNSSKSTSSFPCPAGHFNGFRTVIRPFYLTNSSGVD

SEQ ID NO: 22 - protein, 574 aa, Rattus sp.
MALLWLLSVFLLVPGTQGAKDGDMRLVNGASPSEGRVEIFYRGRWGTLCDNLWNLLDAHVFCRALGYDNATPALNRVAFGPGKGPIMLDEVECTGNESSLANCSSLGWMVSHCRHEKDAGVVCSNDSRGIHILDLSGELPDSLGQIFDSQQDCDLFIQVTGQGHGDLSLCAHTLILRTNPEAQALWQVVGSSVIMRVDAECMPVVRDFLRYFYSRRIEVSMSSVKCLHKLASAYGATELQGYCGRLFVTLLPQDPTFHTPLELYEYAQATGDSVLEDLCVQFLAWMFEPLTQAESWLSVPNALIQALLPKSELAVSSELDLLKAVDQWSTATGASHGDVERLVEQIRFPMMLPQELFELQFNLSLYQGHQALFQRKTMEALEFHTVPLKVLAKYRSLNLTEDVYKPRLYTSSTWSSLLMAGAWSTQKYKYRQFYTYNYGSQSRYSSYQNFQTPQHPSFLFKDKLISWSATYLPTIQSCWNYGFSCTSDELPVLGLTTSSYSNPTIGYENRVLILCGGYSVVDVTSFIGSKAPIPGTQETNSSKTPSLFPCASGAFSSFRVVIRPFYLTNSTDTE

SEQ ID NO: 23 - protein, 577 aa, Murinae gen. sp.
MALLWLLSVFLLVPGTQGTEDGDMRLVNGASANEGRVEIFYRGRWGTVCDNLWNLLDAHVVCRALGYENATQALGRAAFGPGKGPIMLDEVECTGTESSLASCRSLGWMVSRCGHEKDAGVVCSNDTTGLHILDLSGELSDALGQIFDSQQGCDLFIQVTGQGYEDLSLCAHTLILRTNPEAQALWQVVGSSVIMRVDAECMPVVRDFLRYFYSRRIEVSMSSVKCLHKLASAYGATELQDYCGRLFATLLPQDPTFHTPLDLYAYARATGDSMLEDLCVQFLAWNFEPLTQSESWSAVPTTLIQALLPKSELAVSSELDLLKAVDQWSTETIASHEDIERLVEQVRFPMMLPQELFELQFNLSLYQDHQALFQRKTMQALEFHTVPVEVLAKYKGLNLTEDTKYPRLYTSSTWSSLVMASTWRAQRYEYNRYNQLYTYGYGSVARYNSYQSFQTPQHPSFLFKDKQISWSATYLPTMQSCWNYGFSCTSNELPVLGLTTSSYSNPTIGYENRVLILCGGYSVVDVTSFEGSKAPIPTALDTNSSKTPSLFPCASGAFSSFRVVIRPFYLTNSTDMV